(19) United States
(12) Patent Application Publication
(10) Pub. No.: US 2010/0203024 A1
(43) Pub. Date: Aug. 12, 2010

(54) Sickled Erythrocytes, Nucleated Precursors & Erythroleukemia Cells for Targeted Delivery of Oncolytic Viruses, Anti-tumor Proteins, Plasmids, Toxins, Hemolysins & Chemotherapy

(76) Inventors: David S. Terman, Pebble Beach, CA (US); Mark W. Dewhirst, Durham, NC (US)

Correspondence Address: CENTRAL COAST PATENT AGENCY, INC, 3 HANGAR WAY SUITE D, WATSONVILLE, CA, 95076, US

(21) Appl. No.: 12/302,655

(22) PCT Filed: May 29, 2007
(86) PCT No.: PCT/US2007/069869

§ 371 Date: Nov. 26, 2008

Related U.S. Application Data
(60) Provisional application No. 60/809,553, filed on May 30, 2006.
(60) Provisional application No. 60/819,551, filed on Jul. 8, 2006.
(60) Provisional application No. 60/842,213, filed on Sep. 5, 2006.

(51) Int. Cl. A61K 48/00; C12N 5/071; C12N 5/09; C12N 5/095; A61P 35/04

(52) U.S. Cl. 424/93.21; 435/325; 530/350

(57) ABSTRACT

The present invention provides erythrocytes or nucleated erythrocyte precursors from animals or patients with SS or SA hemoglobin or erythroleukemia cells stably transfected with BCAM/Lu which are capable of selectively localizing in tumor vasculature promoting ischemia and occlusion and carrying oncolytic viruses, antitumor proteins, plasmids, toxins and chemotherapy into the tumor milieu. Nucleated erythroid precursors containing SS or SA hemoglobin and transfected with nucleic acids encoding a hypoxia-responsive element and containing nucleic acids encoding expression of oncolytic viruses, superantigens, toxins, viruses, antitumor proteins and chemotherapy are also useful in inducing a potent and specific tumoricidal response. An especially favored carrier is an SS nucleated erythroid precursor transfected with a replication competent oncolytic adenovirus or self-replicating alphavirus expressing a fusogenic membrane glycoprotein or a tumoricidal polypeptide.

### CROSS REFERENCE TO RELATED DOCUMENTS

The present application claims priority to U.S. provisional application Ser. No. 60/809,553 filed on May 30, 2006 and U.S. provisional application Ser. No. 60/819,551 filed on Jul. 8, 2006 and U.S. provisional application Ser. No. 60/842,213 filed on Sep. 5, 2006 and U.S. patent application Ser. No. 10/428,817, filed May 5, 2003 and U.S. provisional application Ser. No. 60/438,686, filed Jan. 9, 2003 and U.S. provisional application Ser. No. 60/415,310, filed on Oct. 1, 2002 and U.S. provisional application Ser. No. 60/406,750, filed on Aug. 29, 2002 and U.S. provisional application Ser. No. 60/415,400, filed on Oct. 2, 2002 and U.S. provisional application Ser. No. 60/406,697, filed on Aug. 28, 2002 and U.S. provisional application Ser. No. 60/389,366, filed on Jun. 15, 2002 and U.S. provisional application Ser. No. 60/378,988, filed on May 8, 2002 and U.S. patent application Ser. No. 09/870,759 filed on May 30, 2001.

### FIELD OF THE INVENTION

The invention is in the fields of genetics and medicine and covers compositions and methods for targeted delivery of anti-tumor agents using sickled erythrocytes, their nucleated precursors, erythroleukemia cells in native state or upregulated for expression of constitutive adhesion molecules and transduced or loaded with hypoxia responsive elements, tumoricidal proteins, toxins, superantigens, hemolysins, oncolytic viruses, chemotherapeutics and anaerobic spores.

### DEFINITIONS

Sickle(d) erythrocytes, SS cells, SS erythrocytes, SS RBCs: Any cell containing an S or SS hemoglobin genes and/or capable of expressing sickled hemoglobin. Sickled cells, sickle hemoglobin variants, SS cells with genetic mutations, SS cells with natural or man-made mutations that increase production/expression of photoreactive porphyrins, SS cells with natural or man-made hemoglobin genes or mutations including but not limited to nucleated precursors and progenitors of each expressing receptors/physical properties capable of binding to tumor cells and/or tumor neovasculature. Also included in this definition are man-made cells into which S or SS hemoglobin genes or mutants or intact or SS homologue proteins have been introduced.

SS cell nucleated precursors: Any nucleated cell containing a natural sickled hemoglobin gene. Also included in this definition are man-made nucleated precursor cells to which an S or SS hemoglobin gene or protein has been added or which has been transfected with S or SS hemoglobin genes.

Sickle hemoglobin variants: Erythrocyte or nucleated erythrocyte precursor/progenitor expressing hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia; any erythrocyte from patients with any form of sickle hemoglobinopathy; any erythrocyte, with or without sickle hemoglobin, their precursors and progenitors expressing receptors capable of binding to tumor cells and/or tumor neovasculature.

Erythroleukemia cells: Mature erythroleukemia cells, their precursors and progenitors expressing receptors capable of binding to tumor cells and/or neovasculature.

### BACKGROUND

The ideal cancer therapeutic should possess the ability to (i) selectively target tumor cells thereby minimizing untoward effects on normal cells, (ii) access both primary tumor and microscopic foci of metastases in vivo, (iii) kill tumor cells it targets or promote oncolysis by other systems. Likewise, desirable features of an effective gene therapy of cancer in vivo are that the gene or gene product is selective and/or specific for tumor cells and either kills tumor cells in which it is expressed or makes these cells susceptible to killing by other agents.

One group of therapeutic agents specific for cancer cells targets molecular systems unique to tumors. These include imatinib mesylate (Gleevec) for chronic myelogenous leukemia and gastrointestinal stromal tumors, gefitinib (Tarceva and Iressa) for non-small-cell lung cancer, and trastuzumab (Herceptin) for breast cancer. Although each drug has serious shortcomings, they share a common mechanism of targeting a specific molecule present in a tumor whose activity drives tumor growth. In addition, tumor cells deficient in the ability to repair breaks in double-stranded DNA such as those lacking either of the BRCA genes are killed by flooding them with breaks using very low doses of the PARP1 inhibitors.

Therapeutic monoclonal antibodies generally target tumor receptors. The most notable are those targeting the receptor tyrosine kinase (TK) signaling or its ligand. Trastuzumab (Herceptin), a recombinant humanized monoclonal antibody against HER-2, increases response rates and improves survival when added to chemotherapy for metastatic HER-2-expressing breast cancer. In combination with adjuvant chemotherapy, it decreases recurrence in women who have early-stage breast cancer with HER-2 overexpression. Another humanized monoclonal antibody, 2C4, blocks dimerization of HER-2 with other ErbB receptors. HER-2 is mutated or overexpressed in lung and colorectal cancer for which anti-HER-2 therapy is also directed. Cetuximab (Erbitux) a chimeric antibody against EGFR has shown activity in combination with chemotherapy in non-small-cell lung cancer, squamous-cell carcinoma of the head and neck, and colorectal cancer. In metastatic, EGFR-positive, chemotherapy-refractory colorectal cancer, cetuximab alone had minimal activity, but when combined with irinotecan it had a 22 percent response rate and modestly increased progression-free and overall survival. ABX-EGF is a humanized anti-EGFR monoclonal antibody with activity as a single agent in phase 2 trials in metastatic renal-cell and colorectal carcinoma. Vascular endothelial growth factor (VEGF) is essential for tumor angiogenesis, and either it or its two receptor TKs (VEGFR-1 and VEGFR-2) are overexpressed in many non-small-cell lung cancers and breast, prostate, renal-cell, and colorectal cancers. In metastatic colorectal cancer, the addition of bevacizumab (Avastin), a humanized anti-VEGF monoclonal antibody, to irinotecan, fluorouracil, and leucovorin led to significant prolongation of survival.

EGFR is overexpressed, mutated, or both in many solid tumors. The anilinoquinazolines Gefitinib (Iressa) and erlotinib (Tarceva) are specific competitive inhibitors of ATP binding by EGFR that were approved by the Food and Drug Administration (FDA) in 2004 for refractory locally advanced or metastatic non-small-cell lung cancer. Gefitinib led to partial responses in 11 to 19 percent of patients with refractory disease in phase 2 trials, whereas erlotinib yielded partial responses in 9 percent of similar patients and improved overall and progression-free survival. However, the addition of gefitinib or erlotinib to chemotherapy in the initial treatment of non-small-cell lung cancer did not yield additional benefit.

Another dysregulated TK is the BCR-ABL which has been implicated as the direct cause of chronic myelogenous leukemia (CML). Imatinib mesylate (Gleevec), a 2-phenylaminopyrimidine compound that is a specific inhibitor of several TKs—namely, ABL, ABL-related gene product (ARG), c-KIT, and PDGF receptor (PDGFR)—induces complete hematologic and cytogenetic remissions in most patients with chronic-phase CML. ABL is also activated by fusion to nucleoporin 214 (NUP214) in 5 percent of T-cell acute lymphoblastic leukemias and to ETV6 (also known as TEL) in rare cases of atypical CML and acute leukemia, both potential targets for imatinib

Peptides containing arginine-glycine-aspartic acid and asparagine-glycine arginine, whose targets are known to be adhesion molecules of the integrin type have been used to target chemotherapy into the tumor. When conjugated to doxorubicin, these peptides substantially improved the therapeutic index of this chemotherapeutic agent in tumor-bearing mice. However, integrins binding the peptide carriers exist not only on tumor cells but also on various endothelial cells and other types of cells thus only a very small portion of the peptide actually deposits in tumors.

While monoclonal antibodies and small molecules have shown specificity for tumor tissue, they are limited to control of relatively small tumor burden. Indeed, monoclonals have not shown a capability of penetrating deeply into the core of many solid tumors. Increasing the affinity of these antibodies for their target tumor cells has not improved and has even worsened the tumor killing effects. Additionally, the antigens/receptors targeted by the monoclonal antibodies are also expressed on non-tumor tissues leading to toxicity which can be significant.

Some viruses have a natural tropism toward tumor cells. Efforts have been directed toward the improving tumoricidal function of these viruses by inducing selective gene replication in tumor cells. To this end, additional strategies include deletion of viral functions dispensable in tumor cells and introduction of tumor-specific promoters into viral genes. For instance, reovirus requires an activated ras pathway for infection, whereas the autonomous parvovirus life cycle is limited to actively replicating cells. Likewise, several natural and engineered mutants of the herpes simplex virus type 1 replicate only in dividing cells. Conditionally replicative adenoviruses (CRAds) have been designed to display oncotropic properties and replicate exclusively in tumor cells. Indeed, self-replicative alphaviruses, adenoviruses and several viral vectors have been engineered to allow gene delivery initially to tumor cells wherein the virus multiplies, then transfers to neighboring tumor cells via a bystander effect thus increasing the oncolytic capacity of these agents.

Several additional strategies have emerged for increasing tumor specificity of oncolytic viruses. First, viral genes that become dispensable in tumor cells can be completely or partially deleted such as the genes responsible for activation the cell cycle through p53 or Rb binding. For example, E1B gene-deleted adenoviruses that replicate preferentially in p53-deficient target cells have been developed for the treatment of various solid tumors. Likewise, mutant viral specific replication in a tumor cell is due to deletion of the retinoblastoma gene (Rb)-binding site of E1a. Replication-competent herpes simplex virus vectors that are unable to make ribonucleotide reductase have been shown to replicate selectively in rapidly dividing cell populations.

Secondly, transcription of viral genes can be controlled by replacing the native viral promoters with tumor-specific or hypoxia-regulated promoters. Replacement of viral promoters with tumor or tissue-specific promoters such as α-fetoprotein (AFP) and prostate-specific antigen (PSA) promoters has been used to drive the adenovirus E1a gene to treat hepatocellular and prostate carcinomas.

Third, signaling networks in tumor cells can be interdicted by viruses. Reovirus and vesicular stomatitis virus (VSV) replicate selectively in cells carrying ras-activating mutations and interferon non-responsive tumors respectively. Mutants defective at other levels such as intracellular trafficking, nuclear import of the viral genome, RNA splicing, nuclear export of RNA, or protein translation are also conceptual candidates. For example, a virus in which the splicing of a viral gene or an interfering stop signal that is regulated like the tumor-associated splice variant of CD44 could be tumor selective.

Additional viruses that have natural core engineered oncolytic properties are given in Kim et al. Nat. Med. 7: 781-187 (2001) and Alemany et al., Nat. Biotechnology 18: 723-730 (2000) which are incorporated by reference in entirety including their references (Tables 1A & 1B). Incorporation of therapeutic transgenes into these replication-competent viral vectors represents another promising method to improve its efficacy/toxicity ratio. Moreover, replicative viruses and vectors can be used as single agents but also in combination with chemotherapy.

The predicted tumor tropism and replication selectivity of replication-competent viruses has not been fully realized due to the emergence of several host interfering factors. The targeting of viruses for tumor tissue after parenteral administration has been inhibited by the presence of natural neutralizing antibodies against the virus in tumor tissue. Indeed, dll520 (ONYX-015) administered systemically against head and neck, pancreatic, ovarian, colorectal, lung, and oral carcinomas brought about no objective tumor responses. Attempts to overcome this problem by incorporating a vascular targeting signal, a receptor ligand and an antibody into the viral capsid have not achieved success. Tissue specific promoters have shown some degree of specificity but have not been able to retain a consistent fidelity in the viral genome. Further attempts to improve both the efficacy and safety of this approach using a similar adenovirus (FGR) construct containing a cytosine deaminase (CD)/herpes simplex virus type-1 thymidine kinase (HSV-1 TK) fusion gene have also had marginal success.

Attempts to target tumors with liposomes and nanoparticles have yet to surmount the problems of disintegration in the bloodstream, uptake by macrophages and Kupffer cells in liver and spleen and inability to pass through the endothelial cell barrier. Pegylated liposomes show reduced uptake by macrophages and a prolonged half-life but still have not exhibited sufficient localization to tumor tissues. Stealth liposomes in which a targeting molecule is attached to a pegylated residue have shown localization to tumors in vivo but to date, no significant therapeutic effects. Once localized to the target cells, liposomes must then traverse the cell membrane. Fusigenic molecules to promote fusion with the cell membrane, penetratin and TAT-mediated translocation, receptor mediated endocytosis have been employed to address this problem but to date have produced no convincing anti-tumor effects (Lasic DD Applications of Liposomes in Handbook of Biological Physics, vol. 1, edited by R Lipowsky & E Sackmann, Elsevier Science, p. 491-519 (1995))

The present invention provides a remedy for these problems of specificity and efficacy. It uses a natural cell, the erythrocyte of sickle cell anemia, its nucleated precursors and sickle hemoglobin variants, erythroleukemia cells which the inventors have observed to have a proclivity to deposit selectively in the tortuous neovasculature of tumors. Indeed, sickled cells show exquisite specificity for tumor microvasculature. In the hypoxemic environment of tumors, SS hemoglobin polymerizes resulting in an increase in membrane rigidity, upregulation of adherence molecules. In this state the SS cells are insufficiently flexible to navigate the channels of the tortuous tumor vasculature. Under these conditions, the cells also upregulate expression of ligands/receptors such as BCAM/Lu, ICAM-4, α₁v4 and CD36 which bind to their cognate receptors/ligands laminin, αvβ3, VCAM-1 and thrombospondin respectively expressed in the tumor vasculature. These interactions promote local hemostasis and vaso-occlusion. The same receptors on sickle cells, their nucleated precursors and sickle hemoglobin variants, erythroleukemia cells are upregulated significantly following adrenergic stimuli such as epinephrine. Such methodology is employed to enhance the targeting of SS erythrocytes, their nucleated precursors, sickle hemoglobin variants, erythroleukemia cells to tumor microvasculature and tumor cells which overexpress laminin and αvβ3. The instant inventors also recognized that the tumor vasculature commonly undergoes oscillations in oxygen tension which predisposes to local ischemia-reperfusion injury leading to release of TNFα that locally upregulates adhesion molecule expression in tumor microvasculature.

The instant inventors also recognized that unlike monoclonal antibodies, these very same SS erythrocytes, their nucleated precursors, sickle hemoglobin variants, erythroleukemia cells are carriers of potent tumoricidal agents into tumors. The inventors contemplated that nucleated SS precursor erythroblasts are equally effective at polymerizing under hypoxemic conditions while nucleation endows them with the ability to be transduced by oncolytic viruses and to carry these viruses specifically into tumor tissue. Indeed, by placing these viruses under control of a hypoxia responsive transcriptional control element (HRE), they are activated selectively in the hypoxemic tumor vasculature rather than normoxemic tissues (see Table 2). The oxygen tension of tumors (as opposed to normal tissues) is in an range appropriate for activation of the HRE especially in concatenated and polymerized form. Replication competent oncolytic viruses lyse the SS cells, their nucleated precursors, sickle hemoglobin variants and erythroleukemia cells resulting in viral shedding into the tumor tissue where they infect surrounding tumor cell via the well established “bystander” effect (i.e., by cell to cell contact). Lysis of the tumor with release of additional oncolytic virus further infects tumors cells specifically resulting in a cascading tumoricidal effect. Self-replicating oncolytic and tumor specific RNA viruses (e.g., the alphavirus family) and adenoviruses optionally incorporating tumoricidal transgenes are particularly preferred. A particularly preferred transgene produced by these viral constructs is the pseudomonas exotoxin A-tumor specific antibody fusion gene. The instant invention therefore exploits the tumor specificity of SS cells to carry tumor specific oncolytic viruses and transgenic tumoricidal molecules specifically into the tumor. As such claimed subject matter is the effective against large and disseminated tumor burden. Indeed, because tumor neoangiogenesis develops with a critical mass of around 75-100 tumor cells, the instant invention targets and eliminates micrometastases as well.

TABLE 1A
Adenoviruses used as oncolytic agents

| Name (serotype) | Basis of tumor-selective propagation | Therapeutic traits |
|---|---|---|
| Ad wild type | None | Oncolysis |
| (various serotypes) | | |
| Ad5/IFN (Ad5) | None | Oncolysis & immuno- |
| | | stimutatory gene therapy |
| A1520 | EIb55kDa-deletk>n abrogates p53 binding | Oncolysis |
| or Onyx015 < Ad2/5) | | |
| AdTK^{RC} | EIbSSkDa-deletion abrogates p53 binding | Oncolytis & suicide |
| | | gene therapy (TK) |
| Ad-5-CD-TKrep | EIb5SkDa-deletion abrogates p53 binding | Oncolysis ft suicide |
| or FGR (ad5) | | gene therapy (CD + TK) |
| AdvEIAdB-F/K20 (Ad5) | EIbSSkDa-deletion abrogates p53 binding | Oncolysis with enhanced |
| | | infectivity |
| AxEIAdB (Ad5) | EIbSSkDa-deletion abrogates p53 binding | Oncolysis & immuno- |
| & AdCAhIL-2 (Ad5) | | slimulatory gene therapy |
| AdD24 (Ad5) | EIa deletion abrogates Rb bhding | Oncolysis |
| CN706 (Ad5) | Regulation of EIa under the PSA promoter | Oncolysis |
| CN763 (Ad5) | Regulation of EIa under the kalikrein 2 promoter | Oncolysis |
| CN764 (Ad5) | Regulation of EIa under the PSA promoter and EIb | Oncolysis |
| | under the kalikrein 2 promoter | |
| CV739 | Regulation of EIa under rat probasin promoter and EI | Oncolysis |
| | bmder human PSA promoter | |
| CV787 | Regulation of EIa under rat probasin | Oncolysis (enhanced |
| | promoter and EIb under human PSA promoter | compared with CV739 due to |
| | | the presence of E |
| AvEIa041 | Regulation of EIa under the AFP promoter | Oncolysis |
| GT5610 (Ad5) + | Regulation of EIa under the AFP promoter | Oncolysis |
| AdHB (Ad5) | | |
| DI337 (Ad5) | None | Oncolysis (enhanced due to EIb- |
| | | 19 kDa deletion) |
| D1316\|Ad5) | The complete deletion of EIa makes this mutant | Oncolysis |
| | dependent on Nrinsic or ML-6-induced EIa- | |
| | like activity | |
| D1118 (Ad5) | The complete deletion of EIb abrogates p53 binding; | Oncolysis |
| | however EIa-induced apoptosts is not inhibited by | |
| | EIb-19 kDa | |

The nucleated sickle erythroblasts and activated erythroleukemia cells of the present invention are a major improvement over monoclonal antibody, liposome and nanoparticle technology since: (i) they exhibit a higher degree of tumor localization, (ii) they penetrate the tumor vasculature more effectively and obstruct or occlude tumor microvessels, (iii) they can be transduced with tumor specific oncolytic viruses such as the self-replicating, RNA alphaviruses and adenoviruses or vectors comprising tumor specific tumoricidal transgenes. Tumor specific oncolytic-oncotropic viruses proliferate in and lyse the SS or erythroleukemia cells and then proceed to infect and kill tumor cells specifically via a bystander effect. Because of the bystander effect and the specificity of oncolytic viruses for tumor cells only a few tumor cells need be infected by the virus in order to initiate a generalized tumoricidal response. (iv) By placing the tumor specific oncolytic viruses under control of concatenated hypoxia-responsive elements, the activation of the tumor specific oncolytic viruses occurs selectively in the hypoxemic microenvironment of the tumor.

TABLE 1B
Replication-Selective Viruses in Clinical Trials

| | | Clinical | Tumor targets | | Cell phenotype allowing |
|---|---|---|---|---|---|
| Parental Strain | Agent | phase | in clinical trials | Genetic alterations | selective replication |
| Engineered | | | | | |
| Adenovirus | [symbol] 1520 [symbol] | I-III | SCCHN | E1B-55-kD gene deletion | Controversial cells lacking p53 function |
| (2/5 chimera) | | | Colorectal | | (for example, deletion, mutation), other? |
| | | | Ovarian | | |
| | | | Pancreatic | E3-10.4/14.5 deletion | |
| Adenovirus | CN706 | I | | E1A expression driven by PSE element | |
| (serotype 5) | CN787 | I | Prostate | E1A driven by rat probasin promoter/ | Prostate cells (malignant, normal) |
| | | | | E1B by PSE/promoter/enhancer | |
| Adenovirus | Ad5-CD/tk-rep | I | Prostate | E1B-55-kD gene deletion | Controversial cells lacking p53 function |
| (2/5 chimera) | | | | Insertion of HSV-tk/CD fusion gene | (for example, deletion, mutation), other? |
| Herpes simplex | G207 | I-II | GBM | ribonucleotide reductase disruption | Proliferating cells |
| virus-1 | | | | (locZ insertion into ICP6 gene) | |
| | | | | neuropathogenesis gene mutation | |
| | | | | (γ-34.5 gene)--both copies | |
| Herpes simplex | NV1020 | I | Colorectal | neuropathogenesis gene mutation | Proliferating cells |
| virus-1 | | | | (γ-34.5 gene)-single copy | |
| Vaccinia virus | Wild-type ± | I | Melanoma | For selectivity: none or [symbol] deletion | Unknown |
| | GM-CSF | | | Immunostimulatory gene (GM-CSF) | |
| | | | | insertion | |
| Non-engineered | | | | | |
| Newcastle | 73-T | I | Bladder | Unknown | Loss of IFN response in tumor cells |
| Disease virus | | | SCCHN | (serial passage on tumor cells) | |
| | | | Ovarian | | |
| Autonomous | H-I | I | | None | Transformed cells |
| parvoviruses | | | | | ↑ proliferation |
| | | | | | ↓ differentiation |
| | | | | | ras, p53 mutation |
| Reovirus | Reolysin | I | SCCHN | None | Ras-pathway activation |
| | | | | | (for example, ras mutation, |
| | | | | | EGFR signalling) |

[symbol]

indicates data missing or illegible when filed

The claimed invention circumvents the problem of viral specific neutralizing antibodies by concentrating the tumor specific oncolytic virus in a relatively avascular tumor bed produced vasoocclusive deposition of SS cells, SS cells or erythroleukemia cells in the tumor vasculature. Under thrombotic and hypoxic conditions in the tumor, the virus is released from the SS or erythroleukemia cells into a relatively avascular tumor and escapes neutralization by viral specific antibodies. The release of virus from the SS cell may be promoted by administration of exogenous erythropoietin which increases intracellular HIF-1 and induces differentiative enucleation of the SS erythroblast. In this scenario, the virus is simultaneously expelled from the cell together with the nucleus.

The inventor also contemplates that the SS cells carry genes encoding immunotoxins such as pseudomonas exotoxin A (PEA), superantigens and other tumor toxins alone or fused to a tumor specific ligand. Preferably these transgenes are integrated into the genome of the tumor specific oncolytic virus or viral vector production and under control of any inducible promoter of which the HRE is preferred. The release of these transgene products into the tumor milieu leads to rapid tumor destruction. The invention is applicable to large established tumors as well as microscopic and metastatic tumor foci in which neoangiogenesis (an early event in tumorigenesis) have developed.

The SS and erythroleukemia cells of the claimed invention differ from other therapies in the field in that they are natural products ideally suited as carriers of tumoricidal agents specifically into tumor cells. They are abundantly available from the large pool of SS patients worldwide and do not require culture conditions for long term maintenance. The native SS erythroblasts and erythroleukemia cells target microvasculature of virtually all tumors without relying on the presence of antibodies or specific signaling molecules. They do not induce the immunosuppression of chemotherapy or the acute toxicity of various toxins. With conventional ABO blood typing SS erythrocytes and erythroleukemia cells can be used as safely in humans as a blood transfusion requiring only one tenth the volume of a conventional unit of blood. Moreover, SS cells do not induce major histoincompatibility-related reactions associated with the use of allogeneic leukocytes. Nor do SS erythroid progenitor cells since they exhibit minimal expression of MHC I and II molecules compared to mature leukocytes and platelets.

### LEGENDS TO FIGURES

FIG. 1. For proteins such as pseudomonas exotoxin A and superantigens and 4-9 copies of the EPO HRE consensus sequence [SEQ ID NO: 39] (CCGGGTAGCTGGCGTACGTGCTGCAG) are inserted into the pβgal-promoter plasmid between SmaI and HindIII sites (CLONTECH) upstream of the simian virus 40 (SV40) or CMV promoter. The expression cassette (nine copies of EPO HRE, SV40 minimal promoter, LacZ gene, and SV40 polyadenylation signal) is cloned into an AAV vector between two inverted terminal repeats to generate the AAVH9LacZ vector.

FIG. 2. AAVH9-Pseudomonas Exotoxin A or AAVH9SEG is generated by replacing LacZ gene in AAVH9LacZ with Pseudomonas exotoxin A or Staphylococcal enterotoxin G respectively.

FIG. 3. HRE4-9 promoter/Sindbis replicon cDNA chimeric construct is shown. (A) Schematic diagram of HRE4-9SINrep/LacZ construct; pro, promoter; SV, Sindbis virus; nsP, nonstructural proteins; SG, subgenomic. (B) The tumoricidal toxin gene has replaced the LacZ gene. In a similar construct, Sindbis structural genes remain intact without substitution.

FIG. 4. Quantification of RBC deposits in the tumor vasculature is shown. Fluorescently-labeled RBCs were infused into the tail vein of nude mice and RBC deposits in tumor vasculature were quantitated. Morphometric analysis showed a 63-fold greater deposition of SS RBCs compared to normal RBCs.

FIG. 5. Quantification of RBC retention by tumor parenchyma is shown. Fluorescently labeled RBCs were infused into the tail vein of nude mice and RBC accumulation in tumor parenchyma was quantitated. RFP sections of the tumor showed diffuse accumulation of SS RBCs in the parenchyma 11-fold greater than normal RBCs

### SUMMARY OF THE INVENTION

The present invention provides erythrocytes with SS hemoglobin, their nucleated precursors, sickle hemoglobin variants, erythroleukemia cells for targeted delivery of tumoricidal agents specifically to the microvasculature of the tumors. Selective generation of tumoricidal agents is promoted by transduction of SS nucleated erythrocyte precursors with the hypoxia responsive promoters or other inducible promoters. These transcriptional regulatory elements in the sickled erythrocytes are activated either by endogenous local conditions (e.g., hypoxia) or by the administration of exogenous agents capable of inducing a specific promoter or enhancer. The promoters are operatively linked to nucleic acids encoding oncolytic viruses, toxins and toxin-antibody fusion proteins or other tumoricidal proteins. Likewise mature sickle cells, sickle cell vesicles and ghosts are loaded with chemotherapy, and anaerobic bacterial spores that are released from the these cells once they have deposited and aggregated in the tumor microvasculature. The present invention contemplates that any oncolytic virus is useful including both replication competent and incompetent optionally containing a transgene encoding any tumoricidal protein, toxin or toxin-antibody fusion protein operatively linked to the HRE or any other inducible promoter in a sickle cell, its nucleated precursors nucleated precursors, sickle hemoglobin variants, erythroleukemia cells. These same SS erythrocytes, their nucleated precursors, sickle hemoglobin variants, erythroleukemia cells may also be transduced with more than one viral constructs containing an oncolytic virus and toxin-antibody fusion gene under control of multiple inducible promoters.

### DESCRIPTION OF THE PREFERRED EMBODIMENTS

### Sickled Erythrocytes and Nucleated Erythroid Precursors for Targeted Delivery of Tumoricidal Agents

Perhaps the most significant problem in therapeutics of cancer is specificity and targeting of anti-tumor agents into tumor tissue while sparing normal tissues. Monoclonal antibodies specific for tumor associated antigen/receptors alone or conjugated to a tumoricidal agent have had some success but, to date, have shown limited effect against large tumor burden, only a modest increase in survival and numerous untoward side effects. Hence, there is a quest for additional agents that can specifically target tumors with more potent tumor killing effects and less morbidity. A recent biopsy of a patient with SA disease and cervical carcinoma showed the selective deposition of sickled erythrocytes in the vasculature of the tumor while the peripheral blood smear had no sickled elements. It was proposed that the hypoxemic environment of the tumor induced polymerization of hemoglobin and sickling in the tumor not seen in the normally oxygenated peripheral blood (Milosevic et al., Gyn Oncol 83; 428-431 (2001)).

Carcinomas are significantly hypoxemic relative to normal tissues (see Table 2). Under these conditions of deoxygenation, SS hemoglobin in erythrocytes from patients with sickle cell anemia polymerizes leading to a sickled morphology. The cells become rigid and are unable to navigate the tortuous and angular microcirculation of the tumor. Due to expression of integrin complex α₄β₁, CD36, BCAM-1/Lu, ICAM-4, SS erythrocytes adhere to the tumor microvascular ligands VCAM-1, platelet thrombospondin, and laminin and αvβ3 integrin respectively. ICAM-4 (LW, CD242) is selectively expressed on sickle but not on normal erythrocytes while BCAM-1/Lu is overexpressed and far more reactive with laminin in sickle cells than in normal red blood cells.

In sickle red blood cells, adhesion to laminin, thrombospondin, fibronectin, and αvβ3 integrin in the vasculature dramatically impacts vaso-occlusion events. The least dense sickle erythrocytes are especially involved in hypoxia-sensitive adherence while secondary trapping of SS4 (dense cells) occurs in post capillary venules. In this way the SS red cells aggregate and obstruct tumor microvessels.

Laminin-α5 is present in endothelial basement membranes subadjacent to the endothelial cells of the vascular wall and is one of the predominant components of the subendothelial matrix in the tumor microvasculature. Laminin and fibronectin are exposed to circulating erythrocytes because the tumor neovasculature contains stretches of vascular matrix, tumor cell canaliculi and sparse endothelium.

Lutheran (Lu) blood group and basal cell adhesion molecule (BCAM) antigens on SS RBCs bind laminin selectively with high affinity under conditions of high shear stress. They exist as two glycoprotein (gp) isoforms Lu and Lu(v13) of 85 and 78 kd respectively and both belong to the IgG superfamily containing identical extracellular domains and differing only by the size of their cytoplasmic tail (Gauthier E, et al., J Biol. Chem. 280:30055-62 (2005)). Sickle red cells bind significant amounts of soluble laminin, whereas normal red cells do not.

BCAM/Lu is the major laminin-binding protein of sickle red cells. Indeed, SS red cells have an average of 67% more BCAM/Lu than normal red cells, and low density red cells from sickle cell disease patients express 40-55% more BCAM/Lu than high density SS red cells (Zen Q et al., J. Biol. Chem. 274: 728-34 (1999); Udani et al., J Clin Invest. 101: 2550-2558 (1998)). Notably, SS erythrocyte adherence to laminin has recently been documented to be more marked than SS erythrocyte adherence to thrombospondin (Hillary C A et al., Blood 87: 4879-4886 (1996)).

In addition, adhesion of sickle cells but not normal erythrocytes to tumor endothelium via laminin α5 and αvβ3 receptors is enhanced by epinephrine acting through the β2-adrenergic receptor, cAMP and protein kinase-dependent signaling pathway. Indeed, exposure to epinephrine for only 1 minute significantly increases sickle erythrocyte adhesion to both primary and immortalized ECs. Thus adrenergic hormones such as epinephrine upregulate BCAM-1/Lu and ICAM-4 expression on sickle erythrocytes and their binding to laminin and αvβ3 receptors respectively improving their ability to localize in the αvβ3- and laminin-rich tumor microvasculature (Hines P C et al., Blood. 101:3281-7 (2003); Zennadi R et al., Blood 104:3774-81 (2004)).

Because of its marked tortuosity and hypoxemia relative to normal tissues, the neovasculature of carcinomas is especially well suited for selective deposition and aggregation of SS erythrocytes. Under hypoxic conditions, inflammatory cytokines such as TNFα, various interleukins and lipid-mediated agonists (prostacyclins) commonly produced by patients with carcinoma also increase the adhesive and hemostatic properties of tumor neovasculature and promote the adherence of SS cells (Table 2). Indeed, the oscillating oxygen tensions noted in the tumor microvasculature (Lanzen et al., Cancer Res., 66: 2219-23 (2006)) predisposes to a hypoxia-reperfusion form of endothelial injury producing increased adherence of SS erythrocytes to the tumor microvasculature.

TABLE 2
Oxygenation of tumors and normal tissues

| | | Median normal PO₂ |
|---|---|---|
| Tumor type | Median PO₂ (pt. no.) | (pt. no.) |
| Glioblastoma | 4.9 (10) | Nd |
| | 5.6 (14) | Nd |
| Head and Neck | 12.0 (30) | 40 (14) |
| Carcinoma | 14.7 (23) | 43.8 (30) |
| | 14.6 (66) | 51.2 (65) |
| Lung Cancer | 7.5 (17) | 38.5 (17) |
| Breast Cancer | 10.0 (15) | Nd |
| Pancreatic Cancer | 2.7 (7) | 51.6 (7) |
| Cervical Cancer | 5.0 (8) | 51.0 (8) |
| | 5.0 (74) | Nd |
| | 3.0 (86) | Nd |
| Prostate Cancer | 2.4 (59) | 30.0 (59) |
| Soft Tissue Sarcoma | 6.4 (34) | Nd |
| | 18.0 (22) | |

### Nucleated Sickle Cells for Transfection of Tumoricidal Agents

Nucleated erythroid precursors or progenitors from patients with sickle cell anemia are the useful in the claimed subject matter. Because they are endowed with nuclei, they are readily transduced with the therapeutic oncolytic viruses and nucleic acids encoding toxins, toxin-tumor specific antibodies, -diabodies, -nanobodies and other therapeutic molecules. The hemoglobin of these cells polymerizes and they undergo characteristic morphological deformation in the form of fine, fragile, elongated spicules consisting of highly organized and tightly aligned hemoglobin fibers in the protruded regions. The nucleated erythroblasts have a larger volume than mature red cells and more dilute hemoglobin which is confined mostly to the cytoplasm. Under partial or complete deoxygenation they behave much like mature SS red cells, i.e., their sickle hemoglobin polymerizes, they deposit and aggregate in the tumor microcirculation.

Nucleated erythroid precursors/progenitors can be readily obtained in abundance from peripheral blood erythrocytes (Fibach E et al., Exp Hematology 26:319-319 (1998); Fibach E et al., Blood 73: 100-103 (1989); Panzenbock B et al., Blood 1998 92:3658-3668; Arcasoy M O & Jiang X Brit. J. Haematol. 130:121-129 (2005)). Peripheral blood (10-20 mL) is drawn from patients with sickle cell anemia. Mononuclear cells isolated by centrifugation on a gradient of Ficoll-Hypaque are cultured according to a two phase liquid culture procedure. In phase 1, the cells are cultured for 7 days in α-minimal essential medium supplemented with 10% fetal calf serum (both from Gifco, Grand Island N.Y.), cyclosporin A (1 ug/mL) (Sandoz, Basel, Switzerland) and 10% conditioned medium collected from bladder carcinoma 5637 cultures. In phase 2, the nonadherent cells are recultured in α-medium supplemented with 30% fetal calf serum, 1% deionized bovine serum albumin, 1×10⁵M 2-mercaptoethanol, 1.5 mM glutamine, 1×10⁻⁶M dexamethasone, and 1 U/mL human recombinant erythropoietin (Ortho Pharmaceutical Co., Raritan N.J.). Cultures are incubated at 37° C. in an atmosphere of 5% CO₂, with extra humidity. Cell morphology is assessed microscopically on cytocentrifuge-prepared slides (Shandon, Cheshire, UK) stained with alkaline benzidine and Giemsa stained with alkaline benzidine and Biemsa.

Nucleated erythroid precursors/progenitors are obtained from bone marrow or erythroid cells or stem cells. They are also obtained from established erythroid and stem cell lines. The desired nucleated progenitor cells are generally CD34+. All of these cells are identified, isolated and enriched using methods well established in the art.

Cell banks are prepared consisting of ABO and Rh typed, nucleated sickle precursor cells, transfected with the appropriate tumoricidal agents under control of the HRE. Cell banks can also include mature SS, SA and other sickle variants cells incorporating anaerobic bacterial spores, Listeria, S. aureus or tumoricidal drugs for use in patients with solid tumors. Thus it is feasible to use nucleated erythroid precursor cells for transfection of HRE and nucleotides encoding tumoricidal agents.

### SS Cells, Nucleated SS Erythroblasts and Erythroleukemia Cells Transduced by Nucleic Acids Encoding Oncolytic Viruses, Tumoricidal Toxins, Toxin-Antibody Proteins, Cytokines Optionally Under Control of the Hypoxia Responsive Element (HRE)

The present invention contemplates the transduction of the SS cells, SS erythroblasts and erythroleukemia cells by oncolytic viruses, plasmids encoding oncolytic viruses, tumoricidal toxins, toxin-antibody proteins, therapeutic antibodies or antibody fragments and cytokines all optionally under the control of the hypoxia responsive element (HRE). The HRE has been reported in the 5′ or 3′ flanking regions of hypoxia responsive molecules VEGF and EPO and phosphoglycerate kinase promoter and several other genes and is indispensable for their hypoxia-induced transcriptional activation. The core consensus sequence is (A/G) CGT (G/C)C (Forsythe, J A et al., Mol. Cell. Biol. 16:4604-4613 (1996); Levy, A P, J. Biol. Chem. 270, 13333-13340 (1995); Gupta, M et al., Blood 96, 491-497 (2000)).

HIF-1, a key transcription factor that binds to HRE, regulates the expression of various hypoxia-responsive molecules such as EPO. HIF-1 is composed of a 120-kDa O₂-regulated β subunit and a 91- to 94-kDa constitutively expressed α subunit. HIF-1 activity depends mainly on the intracellular level of HIF-1α protein, which is regulated in inverse relation to the oxygen concentration by an oxygen-dependent enzyme, prolylhydroxylase 2 (PHD2). Under hypoxic conditions, the α subunit is stabilized because of the lack of proline hydroxylation and accumulates. Stabilized HIF-1α translocates into the nucleus and forms an HIF-1 complex with the almost ubiquitously expressed HIF-1β. The HIF-1 complex binds to hypoxia response elements (HREs) found in enhancers or promoters of hypoxia-inducible genes.

In the present invention, the HRE is used preferably in concatenated form of up to 15 or more repeats (Prentice H et al., Cardiovasc Res. 35:567-74 (1997)). It is activated at tissue oxygen partial pressures of 1% and with more recent improvements in concatenation to 2-2.5%. The latter pO₂ is well within the range of most carcinomas. The HRE can be used with various promoters (complete or minimal) of which the CMV appears to be the most potent under hypoxic conditions. The present invention contemplates that the HRE as a key promoter in the virus or vector used to transduce SS erythrocytes. The inventor contemplates that preferably the HRE is incorporated into SS erythroblasts ex vivo before administration of the erythrocytes to the patient. After the latter cells are administered to a living body with tumor or suspected tumor (microscopic metastases) they localize in tumor microvasculature. Under hypoxemic conditions of the tumor microenvironment, nucleic acids encoding oncolytic viruses and/or tumoricidal transgenes are activated.

The present invention contemplates sickled erythroid precursors optionally containing the HRE found for example in the EPO and VEGF genes to control the transcription of various tumor selective viruses and tumoricidal agents. When this sickled erythrocyte is trapped in hypoxemic tumor microvasculature, the HRE optionally activates the synthesis of the tumoricidal viruses and proteins producing a targeted tumor killing response. The present invention contemplates any inducible promoter operatively linked to nucleic acids encoding any tumoricidal transgenes or constitutive genes including but not limited to tumoricidal viruses, toxins, toxin-tumor specific antibody fusions, cytokines including but not limited to TNFα and IFNγ, lytic agents including but not limited to perforins, granzyme, hemolysins, holotoxins, autolytic toxins and key constitutive enzymes as useful and functional. Inducible promoters and transcriptional control elements useful in the present invention include but are not limited to estrogen and steroid responsive promoters, tetR gene, radiation inducible promoters such as EGR-1, thyroglobulin promoter, albumin promoter, heat responsive promoters, heavy metal responsive promoters, tissue-restricted transcriptional control elements include the α₁-antitrypsin and albumin promoters (hepatocyte-selective), tyrosine hydrolase promoter (melanocytes), villin promoter (intestinal epithelium), glial fibrillary acidic protein promoter (astrocytes), myelin basic protein (glial cells), and the immunoglobulin gene enhancer (B lymphocytes), tumor-selective promoter elements include α-fetoprotein (hepatoma), DF3/MUC1 (breast and other carcinomas), thyroglobulin (thyroid carcinoma), prostate-specific antigen (prostate carcinoma), and carcinoembryonic antigen (breast, lung, and colorectal carcinomas), DF3/MUC1 promoter, Myc/Max family. The erythroid precursor can accept, encode and deliver plasmids of any kind including those expressing tumoricidial viruses and man made virus constructs with tumoricidal activity

The E1B-55 kDa gene-deleted adenovirus is especially desirable since it selectively replicates in and lyses p53-deficient tumor cells. The virus contains a deletion between nucleotides 2496 and 3323 in the E1B region encoding the 55 kDa protein. In addition, a C to T transition at position 2022 in E1B generates a stop codon at the third codon position of the protein. These alterations eliminate expression of the E1B 55 kDa gene in infected cells. Injection of the mutant virus into p53-deficient tumors has shown efficacy in a wide variety of human and animal carcinomas and lymphomas.

In the present invention, HRE is integrated into the E1B-55 kDa gene-deleted adenovirus using methods standard in the art. In this form, expression of the virus is driven by HRE element. The mutated virus HRE-E1B-55 is then used to transduce or infect nucleated SS erythroblasts using standard techniques in the art. When these transformed erythroblasts are administered to tumor bearing hosts, they aggregate in the hypoxic environment of the tumor microvasculature. At this point, viral replication in the erythroblast is activated by the HRE leading to rupture of the erythroblast and shedding of the tumor selective virus into surrounding tumor tissue. The virus will selectively infect and kill p53 deficient tumor cells and is capable of spreading from cell to cell (innocent bystander effect) within the tumor matrix.

In the present invention, an adenovirus vector (preferably conditionally replication competent) contains one or more functional genes required for replication and is optionally placed under the transcriptional control of an inducible promoter such as the HRE. This retards uncontrolled replication and systemic immunization in vivo and reduces undesirable side effects of viral infection. Replication competent self-limiting or self-destructing viral vectors can also be used, as well as replication deficient viral vectors. Any hypoxia inducible promoter is useful, including but not limited to a recombinant promoter comprising a minimal promoter linked to an HIF-1 binding sequence. HRE is one such sequence. HREs have been found in the promoters of several hypoxia inducible genes, including phosphoglycerate kinase-1 (Firth J D et al., Proc Natl Acad Sci 91:6496-500 (1994); Semenza et al., J Biol. Chem. 269:23757-63 (1994)), erythropoietin (Pugh C W et al., Proc Natl Acad Sci 88:10553-7 (1991); Semenza et al., Proc Natl Acad Sci 88:5680-4 (1991)), and VEGF (Liu Y et al., Circ Res. 77:638-43 (1995); Forsythe J A et al., Mol Cell Biol. 16:4604-13 (1996)).

An adenovirus construct is packaged into adenovirus vectors and the prepared virus titer reaches at least 1×10⁶-1×10⁷ pfu/ml. The adenoviral construct is administered in the amount of 1.0 pfu/target cell. Thus, administration of a minimal level of adenoviral construct provides a therapeutic level upon propagation of the virus.

A nucleic acid construct is integrated into a viral genome by ligating the construct into an appropriate restriction site in the genome of the virus. Viral genomes are packaged into viral coats or capsids by any suitable procedure. A suitable packaging cell line is used to generate viral vectors. These packaging lines complement the conditionally replication deficient viral genomes as they usually include the genes which have been put under an inducible promoter deleted in the conditionally replication competent vectors. Thus, the use of packaging lines allows viral vectors of the presently claimed subject matter to be generated in culture.

The present invention contemplates adeno- or self-replicating RNA viral vectors incorporated into SS erythrocytes, their nucleated precursors, sickle hemoglobin variants, erythroleukemia cells and activated by their HREs under hypoxic conditions of the tumor microvasculature leading to hemolysis and shedding of the HRE-containing adeno- or Sindbis virus. By placing the viral gene essential for transcription optionally under the hypoxia responsive promoter element (HRE), viral proliferation is activated under conditions of severe hypoxia present in most tumors and carcinomas in particular. Notably, the HRE also confers these viruses with a natural tropism for tumor cells exhibiting high levels of HIF-1. The ability of this promoter to preferentially direct transcription in hypoxic cells can be assessed by producing a plasmid that contains the promoter operatively linked to several well known fluorescent coding sequences. The HRP-fluorescent marker construct is used to establish stable sublines from tumor cell lines: Cells grown in normoxic conditions do not express the marker whereas cells from stably transduced sublines exposed to hypoxic conditions (with oxygen tension at 0.5 to 1.5%) showed excellent expression of the marker.

Conditional replication competence using the HRE constructs results in selective vector replication in sickle cells localized in the hypoxic tumor microcirculation. An oncolytic virus (preferably tumor selective/specific) linked to the HRE proliferates and hemolyzes the erythrocyte. The virus with an FIRE-viral construct has an affinity for tumor cells with high levels of HIF-1. Other excellent viral constructs such as dll530 and Sindbis viruses by themselves have an affinity for tumor cells deficient in p53 and laminin receptors respectively and are preferably linked to an HRE enhancer. Virus is shed from the burst erythrocyte to infect tumor cells with high levels of HIF-1 (and/or P53 deficiency or laminin receptors). High replication of the vector is achieved in the tumor cells while replication in surrounding non-neoplastic cells is minimal.

For genes that are upregulated in response to hypoxia, wherein the precise sequence that confers hypoxia inducibility is unknown, the responsive sequence can be identified by methods known to the average artisan. Within a candidate promoter region, the presence of regulatory proteins bound to a nucleic acid sequence is detected with variety of methods well known to those skilled in the art (Ausubel et al, ed. Short Protocols in Molecular Biology. New York: Green Publishing Associates and John Wiley & Sons. P. 26-33 (1992)). Briefly, in vivo footprinting assays demonstrate protection of DNA sequences from chemical and enzymatic modification within living or permeabilized cells. Likewise, in vitro footprinting assays show protection of DNA sequences from chemical or enzymatic modification using protein extracts. Nitrocellulose filter-binding assays and gel electrophoresis mobility shift assays (EMSAs) track the presence of radiolabeled regulatory DNA elements based on provision of candidate transcription factors. Computer analysis programs, for example TFSEARCH version 1.3 (Yutaka Akiyama: “TFSEARCH: Searching Transcription Factor Binding Sites”, http://www.rwcp.or.jp/papia/), can also be used to locate consensus sequences of known transcriptional regulatory elements within a genomic region.

A hypoxia inducible promoter is concatamerized, polymerized or combined with additional elements to amplify transcriptional activity and mRNA translation in response to hypoxia. The hypoxia inducible promoter comprises 5-10 tandem copies of the HRE from the human VEGF or EPO gene linked to the CMV minimal promoter or many other promoters well known in the art.

A hypoxia inducible promoter of the presently claimed subject matter is responsive to non-hypoxic stimuli that can be used in combined therapy. For example, the mortalin promoter is induced by low doses of ionizing radiation (Sadekova S et al., Int J Radiat Biol. 72:653-60 (1997)), the hsp27 promoter is activated by 17beta-estradiol and estrogen receptor agonists (Porter J et al., J Mol Endocrinol. 26:31-42 (2001)), the HLA-G promoter is induced by arsenite, and hsp promoters can be activated by photodynamic therapy (Luna M C et al., Cancer Res. 60:1637-44 (2000)). Thus, a hypoxia inducible promoter can comprise additional inducible features or additional DNA elements. Virus administration can be provided before, during, or after radiotherapy; before, during, or after chemotherapy; and/or before, during, or after photodynamic therapy. Moreover, a hypoxia inducible promoter can be derived from any biological source such as the human VEGF or EPO promoter that can direct efficient hypoxia inducible expression as in bovine pulmonary artery endothelial (BPAE) cells (Liu Y et al., Circ Res. 77:638-43 (1995)).

Viral Vectors with Fusogenic Membrane Glycoprotein Expression

A major limitation of tumor-targeted replication competent virus is their relatively poor efficiency in spreading throughout the tumor mass, thereby requiring repeated viral injections administered at multiple sites and over several days. The present invention contemplates oncotropic/oncolytic vectors expressing fusogenic membrane glycoprotein transfected into SS cells, SS progenitors and erythroleukemia cells. Fusogenic proteins are typically derived from enveloped viruses such as HIV1, measles virus (MV-F, MV-H), gibbon ape leukemia virus (GALV), vesicular stomatitis virus G (VSV-G) that use them to fuse membranes, penetrate cells and cause massive syncytium formation and cell death. HIV1, measles virus (MV-F, MV-H) fusion proteins have been inserted in the adenovirus genome. Fusogenic recombinant ad1vector obtained spreads more efficiently through tumor xenografts and is superior to the cytotoxicity caused by wild type adenovirus alone or transfection of tumor cells with HSV-tk or cytosine deaminase suicide genes killing at least 1 log more virus.

To create a fusogenic recombinant adenovirus bicistronic expression cassette from measles virus glycoproteins F and H is used to replace the E1 gene region within the plasmid pAdEasy1 which contains the other essential regions of the adenovirus genome. The E1 gene deletion is critical since FMG fusion in wild type adenovirus is reduced significantly. To drive the expression of the F and H, either the major immediate cytomegalovirus early promoter (CMV promoter) or the adenovirus major late promoter (MLP) is used. The mRNA consists of the coding region for H followed by the encephalomyocarditis virus internal ribosomal entry site (IRES) and the F coding region. Transfection of the E1 deficient-MFG virus into SS cells, SS progenitors or erythroleukemia cells not only enhances selective virus localization in vivo to tumor sites but also confers protection of the virus from neutralizing antibodies in the systemic circulation. Erythroleukemia cells of any kind or their progenitors such as human K562 or murine MEL cells stably transfected with nucleic acids encoding the BCAM/Lu gene are the preferred carriers. The histone deacetylase inhibitor FR901228 enhances adenovirus infection of erythroleukemic cells and is useful in the claimed invention as described by Kitazone M et al., Blood 99: 2248-2251 (2002).

Nucleic acids encoding the FMGs are substituted for the E1 protein in wild type adenovirus or inserted into the heterologous gene site of the replication-competent Sindbis virus. Any other virus with intrinsic oncotropic/oncolytic activity and a functional insertion site for heterologous genes is a candidate for transfection with the FMG. SS erythrocytes, progenitors or erythroleukemia cells are transfected with these viruses using methods well established in the art. Prior to administration to the host, the viral-transfected cells are optionally exposed to a dose of light radiation (100-900 nM, 1-20 min) that produces a delayed t½ hemolysis time of 20-60 minutes after in vivo delivery. After administration, the viral transduced and light radiated cells localize in tumors, hemolyze and shed virus into the surrounding tumor milieu where they infect and kill tumor cells specifically. Optionally, the same oncolytic virus expressing FMG is additionally transfected with nucleic acids encoding HSV-tk which is likewise carried into the tumor by the host SS cell, SS progenitor or erythroleukemia cells. After hemolysis, the virus sheds and infects surrounding tumor cells and induces expression of thymidine kinase. Ganciclovir is then administered which selectively kills the tumor cells expressing thymidine kinase.

| (Measles fusion protein F gene Santibanez, S. et al. J. Gen. Virol. 86, 365-374 | |
|---|---|
| (2005)) | |
| [SEQ ID NO: 40] | |
| 1 | gtgtccatca tggatctcaa ggtgaacgtc tctgccatat tcatggcagt actgttaact |
| 61 | ctccaaacac ccaccggtca aatccattgg ggcaatctct ctaagatagg |
| | ggtagtaggg |
| 121 | ataggaagtg caagctacaa agttatgact cgttccagcc atcaatcatt |
| | agtcataaaa |
| 181 | ttaatgccca atataactct cctcaataac tgcacgaggg tagagattgc |
| | agaatacagg |
| 241 | agactactga gaacagtttt ggaaccaatt agagatgcac ttaatgcaat |
| | gacccagaat |
| 301 | ataagaccgg ttcagagtgt agcctcaagt aggagacaca agagatttgc |
| | gggagttgtc |
| 361 | ctggcaggtg cggccctagg cgttgccaca gctgctcaga taacagccgg |
| | cattgcactt |
| 421 | caccagtcca tgctgaactc tcaagccatc gacaatctga gagcgagcct |
| | ggaaactacc |
| 481 | aatcaggcaa ttgaggcaat cagacaagca gggcaggaga tgatattggc |
| | tgttcagggt |
| 541 | gtccaagact acatcaataa tgagctgata ccgtctatga accaactatc |
| | ttgtgattta |
| 601 | atcggccaga agctagggct caaattgctc agatactata cagaaatcct |
| | gtcactattt |
| 661 | ggccccagct tacgggaccc catatctgcg gagatatcta tccaggcttt |
| | gagctatgcg |
| 721 | cttggaggag atatcaacaa ggtgttagaa aagctcggat acagtggagg |
| | tgacttactg |
| 781 | ggcatcttag agagcagggg aataaaagcc cggataactc acgtcgacac |
| | agagtcctac |
| 841 | ttcattgtac tcagtatagc ctatccgacg ctgtccgaga ttaagggggt |
| | gattgtccac |
| 901 | cggctagagg gggtctcgta caacataggc tcccaagagt ggtataccac |
| | tgtgcccaag |
| 961 | tatgttgcaa cccaagggta cctcatctcg aattttgatg aatcatcgtg |
| | tactttcatg |
| 1021 | ccagagggaa ctgtgtgcag ccaaaatgcc ttgtacccaa tgagtcctct |
| | gctccaagaa |
| 1081 | tgcctccggg ggtccaccaa gtcctgtgct cgtacactcg tatccgggtc |
| | ttttgggaac |
| 1141 | cggttcattt tatcacaagg gaatctaata gccaattgtg catcaatcct |
| | ttgcaagtgt |
| 1201 | tacacaacag gaacgatcat taatcaagac cctgacaaga tcctaacata |
| | cattgctgcc |
| 1261 | gatcactgcc cggtggtcga ggtgaacggc gtgaccatcc aagtcgggag |
| | caggagatat |
| 1321 | ccggacgctg tgtacttgca cagaattgac ctcggtcctc ccatatcatt |
| | ggagaggttg |
| 1381 | gacgtaggga caaatctggg gaatgcaatt gccaagttgg aggacgccaa |
| | ggaattgttg |
| 1441 | gagtcatcgg accagatatt gaggagtatg aaaggcttat cgagcactag |
| | catagtttac |
| 1501 | atcctgattg cagtgtgtct tggagggctg atagggatcc ccgctttaat |
| | atgttgctgc |
| 1561 | agggggcgtt gtaacaaaaa gggagaacaa gttggtatgt caagaccagg |
| | cctaaagcct |
| 1621 | gatcttacag gaacatcaaa atcctatgta aggtcgctct ga |
| (Measles fusion glycoprotein F1 and F2 Richardson, C., Virology 15,508-523 | |
| (1986)) | |
| [SEQ ID NO: 41] | |
| 1 | mglkvnvsai fmavlltlqt ptgqihwgnl skigvvgigs asykvmtrss hqslviklmp |
| 61 | nitllnnctr veiaeyrrll rtvlepirda lnamtqnirp vqsvassrrh |
| | krfagvvlag |
| 121 | aalgvataaq itagialhqs mlnsqaidnl raslettnqa ieairqagqe |
| | milavqgvqd |
| 181 | yinnelipsm nqlscdligq klglkllryy teilslfgps lrdpisaeis |
| | iqalsyalgg |
| 241 | dinkvleklg ysggdllgil esrgikarit hvdtesyfiv lsiayptlse |
| | ikgvivhrle |
| 301 | gvsynigsqe wyttvpkyva tqgylisnfd essctfmpeg tvcsqnalyp |
| | mspllqeclr |
| 361 | gstkscartl vsgsfgnrfi lsqgnlianc asilckcytt gtiinqdpdk |
| | iltyiaadhc |
| 421 | pvvevngvti qvgsrrypda vylhridlgp pislerldvg tnlgnaiakl |
| | edakelless |
| 481 | dqilrsmkgl sstsivyili avclggligi palicccrgr cnkkgeqvgm |
| | srpglkpdlt |
| 541 | gtsksyvrsl |

### Viral Vectors Targeting the Tumor Neovasculature

Targeting specifically the blood vessels of the tumor instead of the tumor itself is attractive since they are within easy reach of viral carrier SS cell, SS progenitor or erythroleukemia cells that deposit preferentially in tumor endothelium. Moreover, the killing of one tumor endothelial cell is known to support the nutritional needs of approximately 100 tumor cells. Viruses such as the adenovirus expressing flt and endoglin genes target endothelium and are capable of infecting it. Similarly, the virus can contain nucleic acids encoding an immunoglobulin specific for epitopes expressed on the tumor endothelium such as VEGF or BCAM/Lu or combinations thereof.

The present invention contemplates the use of replication-selective oncolytic viruses targeting the tumor endothelium. In order to transcriptionally target the dividing endothelial cell, promoters of genes with specificity for these cells are utilized. Flk-1 (also called KDR and VEGFR-2) is a high-affinity tyrosine kinase receptor for the angiogenic growth factor, VEGF. Flk-1 is an endothelial-cell specific gene which in contrast to other endothelial genes expression is absent in most vascular beds in the adult organism, but is highly induced in the newly formed blood vessels in a variety of human tumors. Endoglin (CD105) a cell surface component of the TGF-β receptor complex, is also preferentially expressed by tumor endothelial cells especially in the endothelium of the tumor edges, where active cell division occurs.

Two novel adenoviruses, Ad.Flk-1 and Ad.Flk-Endo, which induce the expression of adenoviral E1A and E1B genes selectively in dividing endothelial cells by Flk-1 and endoglin promoters respectively are useful in this invention. Ad.Flk-1 and Ad.Flk-Endo possess significant differential replication ratios in Flk-1 and endoglin positive cells of 30 and 600 fold respectively, as compared with cells where these genes are not expressed. Ad.Flk-1 and Ad.Flk-Endo also cause selective cytotoxicity as they killed Flk-1 and endoglin positive HUVECs as efficiently as wild-type virus.

In the present invention SS cells, progenitors and erythroleukemia cells stably transfected with BCAM/Lu or other receptor whose cognate ligand is situated in the tumor neovasculature are transfected with the Ad.Flk-Endo vector. Production of these vectors is given below. The SS cells, SS progenitors and erythroleukemia cells (10⁶-10¹¹) are administered parenterally in vivo to tumor bearing hosts. These cells localize in the tumor neovasculature and undergo viral- and/or photo-induced hemolysis as described below. The transcriptionally targeted virus shed from the SS cells, progenitors and erythroleukemia cells selectively infects the tumor endothelial cells resulting in selective lysis.

### Construction of Ad.Flk-1 and Ad.Flk-Endo

Plasmid pXC1, which contains human adenovirus 5 sequences from by 22 to 5790, is purchased from Microbix (Microbix Biosystems, Toronto, Canada). For insertion of the Flk-1 enhancer/promoter a unique Agel site is created in the adenovirus E1 A promoter of pXC1 by overlapping PCR as follows. The first primer pair [SEQ ID NO: 42] (TCGTCTTCAAGAATTCTCATG (sense) and [SEQ ID NO: 43] TTTCAG TCACCGGTGTCGGA (antisense)) produced a PCR fragment from the unique EcoRI site in the PBR322 backbone of pXC1 to the new Agel site at position 547. The second primer pair [SEQ ID NO: 44] (TCCGACACCGGTGACTGAAA (sense) and [SEQ ID NO: 45] GCATTCTCTAGACACAGGTG (antisense) produced a PCR fragment from the new Agel site to a unique Xbal site at position 1339. Combining equal amounts of the two PCR products, a third PCR is performed with the two outside primers, cut with EcoRI and Xbal, and cloned into similarly cleaved pUC19 to yield pUCE1 A. This plasmid is cut with unique enzymes Sacll at position 357 and Agel at position 547 to delete the endogenous adenovirus E1A promoter.

To amplify the Flk-1 enhancer and promoter liver tissue from a BALB/c mouse is homogenized, and DNA is extracted using DNeasy Tissue Kit (Qiagen, Valencia, Calif.). A 923-bp Flk-1 promoter element is amplified using specific primers based on published sequence (SEQ ID NO: 46): ATTTAGCGGCCGCagttcacaaccgaaatgtcTTC (sense primer with Notl linker) and (SEQ ID NO: 47) AGTTTA CCGGTATCCTGCACCTCGCGCTG (antisense primer with Agel linker).⁴⁰ A 510-bp Flk-1 enhancer element is amplified using specific primers based on published sequence (SEQ ID NO: 48): TCCCCGCGGTAAATGTGCTGT-CTTTAGAAG (sense primer with SflcII linker) and (SEQ ID NO: 49) AATATGCGG CCGCTCCAATAGGAAAGCCCTIC (antisense primer with Notl linker). The promoter and enhancer fragments are cut with Notl-Agel and SacU-Notl, respectively, and cloned into similarly cut pUCE1A to yield pUCE1A-Flk1. The modified E1A fragment containing the Flk-1

To construct pFlk-Endo a unique Blpl restriction is created in pFlk-1 by overlapping PCR as follows. The first primer pair (SEQ ID NO: 50) (TCACCTGTGTCTA GAGAATGC (sense) and (SEQ ID NO: 51) GTAACCAAGCTTAG CCCACG (antisense)) produced a PCR fragment from the unique Xbal site of pFlk-1 to the new Blpl site at position 1690 of adenovirus sequence. The second primer pair (SEQ ID NO: 52) (CGTGGGCTAAGCTTGGTTAC (sense) and (SEQ ID NO: 53) CCA GAAAATCCAGCAGGTACC (antisense) produce a PCR fragment from the new Blpl site to a unique Kpnl site of pFlk-1. Combining equal amounts of the two PCR products, a third PCR is performed with the two outside primers, cut with Xbal and Kpnl, and cloned into Xbal-Kpril digested pFlk-1 to yield pFlk-Blpl, which now has a unique Blpl site before the transcription start site of E1B.

To amplify the endoglin promoter human DNA is extracted from whole blood using QIAamp DNA Blood Mini Kit (Qiagen). A 741-bp endoglin promoter is amplified using specific primers based on published sequence (SEQ ID NO: 54): GATCATGCTAAGCGATCCC AGCGCTACCATCTTC (sense primer with Blpl linker) and (SEQ ID NO: 55) TATAATGCTTAGCGTGGGGGCCTGTGCGCTGG (antisense primer with Blpl linker). The promoter fragment is cut with Blpl and cloned into similarly cleaved pFlk-1-BlpI to yield pFlk-Endo. The sequence of all PCR fragments in pFlk-1 and pFlk-Endo are verified by sequencing.

Recombinant adenoviruses Ad.Flk-1 and Ad.Flk-Endo are prepared by co-transfecting 293 cells with plasmids pFlk-1 and pFlk-Endo with backbone plasmid pBHGlO (Microbix), which contains an E1 deletion of by 188-1339 and an E3 deletion of by 28133-3081. Recombinant adenovirus is isolated from a single plaque, expanded in 293 cells and purified by double cesium gradient ultracentrifugation. The viral particles are measured by optical absorbance at 260 nm, and the plaque-forming units (p.f.u.) are determined by standard agarose-overlay plaque assay on 293 cells. The genome lengths of Ad.Flk-1 and Ad.Flk-Endo are 97% and 99% of wild-type Ad5, respectively.

### Tumoricidal Transgenes

In order to more efficiently kill a cell that contains an adenovirus vector a transgene is provided. A transgene comprises a therapeutic gene, including, but not limited to a tumor suppressor gene, an apoptosis-inducing gene, an anti-angiogenic gene, a suicide prodrug, converting enzyme gene, a bacterial toxin gene, an antisense gene, a tumor suppressor gene, an immunostimulatory gene, or combinations thereof. A “transgene” A transgene includes a gene that is partly or entirely heterologous (i.e., foreign) to the organism from which the cell was derived, or can be a nucleotide sequence identical or homologous to a gene already contained within the cell.

The transgene is encoded by a conditionally replication competent adenovirus vector. Since the number of exogenous nucleotides that can be efficiently packaged into an adenovirus virion is about 2000 base pairs, a conditionally replication competent adenovirus vector can comprise a transgene of no more than about 1.4-1.6 kilobases (kb), in addition to the essential promoter and polyadenylation sequences. Transgenes larger than this are provided by other mechanisms.

Transgenes may also be delivered by replication-competent vectors which may be noncytopathic. Transgenes comprise nucleic acids encoding a polypeptide having a therapeutic biological activity. Exemplary therapeutic polypeptides include but are not limited to TNFα, IFNγ, and immunostimulatory molecules, various cell toxins alone or fused or conjugated to a tumor targeting agent, tumor suppressor gene products/antigens, suicide gene products, and anti-angiogenic factors or prodrug-activating enzymes that release well-defined cytotoxins on reduction in hypoxic cells such as nitrobenzyl phosphoramidate mustards, nitroheterocyclic methylquaternary salts, cobalt(III) complexes and indoloquinones (see Mackensen et al., Cytokine Growth Factor Rev. 8:119-28 (1997); Walther et al., Mol. Biotechnol. 13:21-8 (1999); Kirk et al., Hum Gene Ther. 11:797-806 (2000)) and references cited therein. In addition the transgene can express a ligand such as hergulin which binds overexpressed human epidermal growth factor receptor (HER). The RNA alphaviruses exemplified by the Sindbis virus which selectively targets overexpressed laminin receptors on tumor cells may be incorporated into sickled erythrocytes or erythroblasts optionally under control of the HRE or promoters. Upon lysis of the sickled erythrocyte by the virus, free virus is shed into the tumor microenvironment where it can selectively target surrounding tumor cells. A suicide gene encoding a protein that causes cell death directly, for example by inducing apoptosis, is referred to as an “apoptosis-inducing gene” and includes but is not limited to TNFα (Idriss et al., Microsc Res Tech. 50:184-95 (2000)), TRAIL (Srivastava Neoplasia 3:535-46 (2001)), Bax, and Bcl-2 (Shen et al., Adv Cancer Res. 82:55-84 (2001)). Other genes that encode proteins that kill cells directly include bacterial toxin genes, which are normally found in the genome of certain bacteria and encode polypeptides (i.e. bacterial toxins) that are toxic to eukaryotic cells. Bacterial toxins include but are not limited to diphtheria toxin, pseudomonas exotoxin A and superantigens (Frankel et al., Curr Opin Investig Drugs 2:1294-301 (2001)). The list of superantigens useful in this construct is given in the instant application with a preference for the staphylococcal enterotoxins of the enterotoxin gene complex (egc).

Additional suicide genes encode a polypeptide that converts a prodrug to a toxic compound. Such suicide prodrug converting enzymes include, but are not limited to the HSV-tk polypeptide, which converts ganciclovir to a toxic nucleotide analog (Freeman et. al., Semin Oncol. 23:31-45 (1996); cytosine deaminase, which converts the non-toxic nucleotide analog 5-fluorocytosine into a toxic analog, 5-fluorouracil (Yazawa et al. World J Surg 26:783-9 (2002); and cytochrome p450, which converts certain aliphatic amine N-oxides into toxic metabolites (Patterson L H Curr Pharm Des. 8:1335-47 (2002). Additionally, a suicide gene can encode a polypeptide that interferes with a signal transduction cascade involved with cellular survival or proliferation. Such cascades include, but are not limited to, the cascades mediated by the Flt1 and Flk1 receptor tyrosine kinases (reviewed in Klohs et al., Curr Opin Oncol. 9:562-8 (1997)). Polypeptides that can interfere with Flt1 and/or Flk1 signal transduction include, but are not limited to, a soluble Flt1 receptor (s-Flt1; Shibuya M Int J Biochem Cell Biol. 33:409-20 (2001) and an extracellular domain of the Flk-1 receptor (ex-Flk1; Lin P et al., Cell Growth Differ. 9:49-58 (1998)).

In another embodiment, sickled erythrocytes, erythroblasts or erythroleukemia cells are infected with two different adenovirus vectors, one a conditionally replication competent vector comprising an oncolytic viral gene under the transcriptional regulation of an HRE, and the other a replication deficient adenovirus vector comprising a transgene such as α-hemolysin to lyse the SS cell. The use of a combination approach offers advantages in that a conditionally replication competent adenovirus has a capacity for a transgene of only about 2 kb (if the foreign promoter is small) to carry transgenes. Thus, the capacity of an adenovirus vector to carry transgenes, which in many cases exceed 2 kb, can be expanded. With the use of a replication-deficient virus in conjunction with the conditionally replication competent virus, the ability to deliver transgenes can be significantly expanded. In the case of a first generation E1, E3 defective adenovirus vectors, the capacity will be about 8 kb. In the case of third generation gutless vectors, the capacity will reach approximately 37 kb. Construction of gutless vectors is well described in the art.

In another construct, tumor specific viral replication the E1B or E1A is placed under control of a tissue specific promoter or element such as PSE, PSA, D3/MUC-1 promoter, albumin enhancer promoter and the like. In addition, viruses that are engineered to delete functional region(s) necessary for replication in normal cells such as the di 1530 (E1B-55 kD deletion), G207-HSV1 (ribonucleotide reductase disruption), 1716-HSV (γ34.5 deletion) but are expendable in tumor cells such as Ad-Δ24(Ad) and d/922-047(Ad) with deletion of E1A:CR2-pRB family binding site, KD1, KD3 (Ad) with deletion of E1ACR1 and CR2-p300, pRB binding regions, PV1 (RIPO) with 5′-IRES replaced with HRV2 are useful Likewise, viruses that are engineered to express tumor specific ligands or receptors or inherently tumor selective viruses such as NDV (73T) autonomous parovirus (H1) that target tumor interferon resistant tumors reovirus and Sindbis virus that target Ras pathways and laminin receptors respectively are useful.

The HRE-E1B-55 is also encapsulated within sickled erythrocyte ghosts prepared by methods described below. These viral infected ghosts are administered to tumor bearing hosts where under hypoxemic conditions of the tumor microvasculature they aggregate, produce lytic virus which first lyses the erythroblast and then spreads cell to cell to infect surrounding tumor cells. Additional oncolytic viruses useful in this fashion include but are not limited to herpes simplex, adenoviruses, vaccinia, Newcastle Disease virus, autonomous parvoviruses, reovirus and various other oncolytic viruses with tumor specificity that can be used to transfect sickle cells are described in Kirn, D et al., Nat. Med. 7:781-7 (2001) incorporated by reference in entirety including references cited therein. Similarly, anaerobic bacterial spores such as Clostridia novyi can be encapsulated in sickled erythrocyte ghosts to carry them into tumor microvasculature where they induce a tumoricidal response (Dang et al., Proc. Natl. Acad. Sci. 89: 15155-15160 (2001)).

Additional oncolytic viruses are useful to infect sickled nucleated precursor cells which have been transduced with nucleic acids encoding hemolysins, optionally placed under the hypoxia response element as described herein. Staphylococcal alpha hemolysin and Listeria hemolysin are excellent candidates for this purpose but many other hemolysins are useful as well. The amino acid sequences of alpha hemolysin and Listeria hemolysin are given below:

When entering the hypoxic tumor microcirculation, the sickled erythrocyte adheres to the tumor vasculature and the HRE is activated inducing the formation of nucleotides encoding the hemolysins which hemolyze the erythrocyte releasing oncolytic virus into the tumor site. Sickle cell deposition in tumor vessels leads to reduced SS cell velocity, upregulation of endothelial VCAM-1, TNFα, and p-selectin, trapping of additional sickled cells and micro-occlusion of the tumor microvasculature.

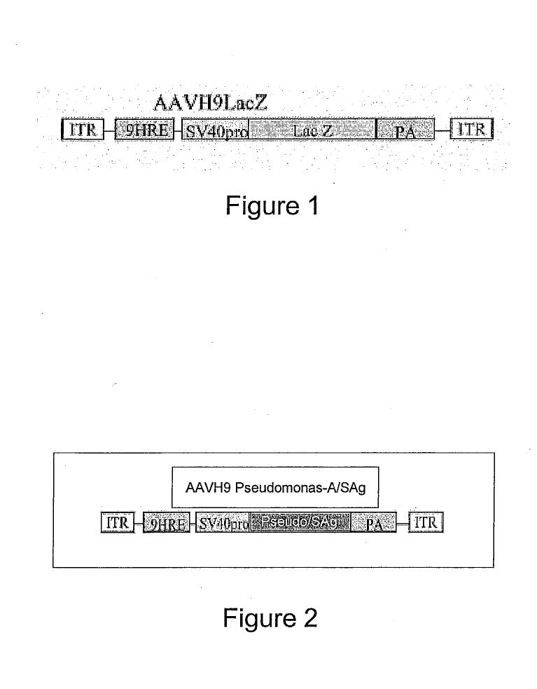

For proteins such as Pseudomonas exotoxin A and superantigens, 4-9 copies of the EPO FIRE consensus sequence (SEQ ID NO: 39) (CCGGGTAGCTGGCGTACGTGCTGCAG) are optionally inserted into the pβgal-promoter plasmid between SmaI and HindIII sites (CLONTECH) upstream of the simian virus 40 (SV40) or CMV promoter. The expression cassette (nine copies of optional EPO HRE, SV40 minimal promoter, LacZ gene, and SV40 polyadenylation signal) is cloned into an AAV vector between two inverted terminal repeats to generate the AAVH9LacZ vector as shown in FIG. 1.

AAVH9 Pseudomonas Exotoxin A or AAVH9 SEG is generated by replacing LacZ gene in AAVH9LacZ with Pseudomonas exotoxin A or Staphylococcal enterotoxin G respectively as shown in FIG. 2.

AAV vectors are prepared by using the three-plasmid cotransfection system. AAV vector is cotransfected with two helper plasmids (provided by Avigen, Alameda, Calif.) into sickled erythroid precursors by the calcium phosphate precipitation method. One helper plasmid, pLadeno5, has the adenoviral VA, E2A, and E4 regions that mediate AAV vector replication. The other, pHLP19, has AAV rep and cap genes.

The HRE is optionally fused to various nucleic acids encoding tumoricidal proteins including but not limited to superantigens (preferably staphylococcal enterotoxins G, I, M, N, O), Pseudomonas exotoxins (exotoxin A being the best characterized), verotoxins and/or subunits, diptheria toxin, pertussis toxin, complement membrane attack complex, perforins, holins, S. aureus autolysins, granzymes, tumor specific antibodies, chemokines, cytokines and chemoattractants. Likewise a hemolysin such as S. aureus alpha toxin, Listeria or E. Coli hemolysin are fused to the HRE to facilitate the internal lysis of the SS erythroid precursors under hypoxic conditions.

Pseudomonas exotoxin A (PEA) is a potent bacterial toxin composed of three major domains: (i) domain Ia (amino acids 1-252) is the cell binding domain; (ii) domain II (amino acids 253-364) is responsible for translocation into the cytosol; and (iii) domain III (amino acids 400-613) ADP-ribosylates elongation factor 2, arresting protein synthesis and causing cell death, and also contains the COOH-terminal sequence (SEQ ID NO: 56) REDLK, which directs the endocytosed toxin to the ER. Domain Ib (amino acids 365-399) is a minor domain, and its function is unknown. PE38 is a modified form of PE in which all of domain Ia and amino acids 365-380 of domain Ib have been deleted.

Recombinant Pseudomonas exotoxin molecules display cytotoxic activity. The cytotoxicity may be retained even if a domain or a portion thereof such as the amino terminal end of domain Π is deleted. This molecule may be linked or fused to other targeting or ligand binding agents specific for target cells so that the cytotoxicity is targeted to desired cells.

Native PEA has the amino acid sequence set forth below. It is used as a frame of reference for variants of this molecule. Other common references are used herein to indicate deletions or substitutions to a sequence using the sequence below as the reference (U.S. Pat. No. 5,602,095)

| (SEQ ID NO: 57) | |
|---|---|
| 1 | aeeafdlwne cakacvldlk dgvrssrmsv dpaiadtngq gvlhysmvle ggndalklai |
| 61 | dnalsitsdg ltirleggve pnkpvrysyt rqargswsln wlvpighekp snikvfihel |
| 121 | nagnqlshms piytiemgde llaklardat ffvrahesne mqptlaisha gvsvvmaqaq |
| 181 | prrekrwsew asgkvlclld pldgvynyla qqrcnlddtw egkiyrvlag npakhdldik |
| 241 | ptvishrlhf peggslaalt ahqachlple tftrhrqprg weqleqcgyp vqrlvalyla |
| 301 | arlswnqvdq virnalaspg sggdlgeair eqpeqarlal tlaaaeserf vrqgtgndea |
| 361 | gaanadvvsl tcpvaageca gpadsgdall ernyptgaef lgdggdvsfs trgtqnwtve |
| 421 | rllqahrqle ergyvfvgyh gtfleaaqsi vfggvrarsq dldaiwrgfy iagdpalayg |
| 481 | yaqdqepdar grirngallr vyvprsslpg fyrtsltlaa peaageverl ighplplrld |
| 541 | aitgpeeegg rletilgwpl aertvvipsa iptdprnvgg dldpssipdk eqaisalpdy |
| 601 | asqpgkppre dlk |

A useful PEA molecule is one in which domain Ia is deleted and no more than the first 27 amino acids have been deleted from the amino terminal end of domain II. This substantially represents the deletion of amino acids 1 to 279. The cytotoxic advantage created by this deletion is decreased if the following deletions are made: 1-281; 1-283; 1-286; and 314-380. In addition, the PE molecules can be further modified using site-directed mutagenesis or other techniques known in the art, to alter the molecule for particular desired application. Means to alter the PE molecule in a manner that does not substantially affect the functional advantages provided by the PE molecules described here can also be used and such resulting molecules are intended to be covered herein.

To maximize the cytotoxic properties of a preferred PE molecule, several modifications to the molecule are recommended. An appropriate carboxyl terminal sequence to the 5 recombinant molecules is preferred to translocate the molecule into the cytosol of target cells. Amino acid sequences which have been found to be effective include, REDLK (SEQ ID NO: 55) (as in native PE), REDL (SEQ ID NO: 58) or KDEL (SEQ ID NO: 59), repeats of those, or other sequences that function to maintain or recycle proteins into the endoplasmic reticulum.

Deletions of amino acids 365-380 of domain Ib does not result in loss of activity. Further, a substitution of methionine at amino acid position 280 in place of glycine to allow the synthesis of the protein to begin and of serine at amino acid position 287 in place of cysteine to prevent formation of improper disulfide bonds is beneficial.

Useful ligand binding agents include all molecules capable of reacting with or otherwise recognizing or binding to a receptor on a target cell. Examples of such binding agents include, but are not limited to, antibodies, growth factors such as TGFα, IL2, IL4, IL6, IGF1 or CD4, lymphokines, cytokines, hormones and the like which specifically bind desired target cells.

Antibodies include various forms of modified or altered antibodies, such as an intact immunoglobulin, an Fv fragment containing only the light and heavy chain variable regions, a Fab or (Fab)′₂ fragment containing the variable regions and parts of the constant regions, a single-chain antibody (Bird et al., Science 242, 424-426 (1988); Huston et al., Nat. Acad. Sci. USA 85, 5879-5883 (1988)). The antibody may be of animal (especially mouse or rat) or human origin or may be chimeric (Morrison et al., Proc Nat. Acad. Sci. USA 81, 6851-6855 (1984)) or humanized (Jones et al., Nature 321. 522-525 (1986), and published UK patent application #8707252). Methods of producing antibodies suitable for use in the present invention are well known to those skilled in the art and can be found described in such publications as Harlow & Lane, Antibodies: A Laboratory Manual, Cold Spring Harbor Laboratory, (1988).

The recombinant PE molecules may be fused to, or otherwise bound to a ligand binding agent by recombinant methods well known and available to those in the art. Production of various immunotoxins is well-known within the art and can be found, for example in “Monoclonal Antibody-Toxin Conjugates: Aiming the Magic Bullet,” Thorpe et al., Monoclonal Antibodies in Clinical Medicine, Academic Press, pp. 168-190 (1982) and Waldmann, Science, 252:1657 (1991), both of which are incorporated by reference. To use the recombinant PE molecules with an antibody, a form of the PE molecule with cysteine at amino acid position 287 is preferred to couple the toxin to the antibody or other ligand through the thiol moiety of cysteine. The PE molecules may also be fused to the ligand binding agent by recombinant means such as through the production of single chain antibodies in E. coli. The genes encoding protein chains may be cloned in cDNA or in genomic form by any cloning procedure known to those skilled in the art. See for example Sambrook et al., Molecular Cloning: A Laboratory Manual, Cold Spring Harbor laboratory, (1989). It is desirable to insert the ligand binding agent at a point within domain III of the PE molecule, particularly for smaller agents such as TGFα (transforming growth factor α). Most preferably the ligand binding agent is fused between about amino acid positions 607 and 604 of the PE molecule. This means that the ligand binding agent is inserted after about amino acid 607 of the molecule and an appropriate carboxyl end of PE is recreated by placing amino acids about 604-613 of PE after the binding agent. Thus, the ligand binding agent is inserted within the recombinant PE molecule after about amino acid 607 and is followed by amino acids 604-613 of domain III V_{L} and V_{H} regions from a desired antibody may also be inserted in a single chain form within domain III. Binding agents may also be inserted in replacement for domain is as has been accomplished in what is known as the TGFα/PE40 molecule (also referred to as TP40).

Those skilled in the art will realize that additional modifications, deletions, insertions and the like may be made to the ligand binding agent and PE genes. Especially, deletions or changes may be made in PE or in a linker connecting an antibody gene to PE, in order to increase cytotoxicity of the fusion protein toward target cells or to decrease nonspecific cytotoxicity toward cells without antigen for the antibody. All such constructions may be made by methods of genetic engineering well known to those skilled in the art (see, generally, Sambrook et al., supra) and may produce proteins that have differing properties of affinity, specificity, stability and toxicity that make them particularly suitable for various clinical or biological applications.

Fusion proteins of the invention including PE molecules may be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli this includes a promoter such as the T7, tip, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences. The plasmids of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant fusion proteins can be purified according to standard procedures of the art, including ammonium sulfate precipitation, affinity columns, column chromatography, gel electrophoresis and the like (see, generally, R. Scopes, Protein Purification, Springer-Verlag, N.Y. (1982)). Substantially pure compositions of at least about 98 to 99% are preferred.

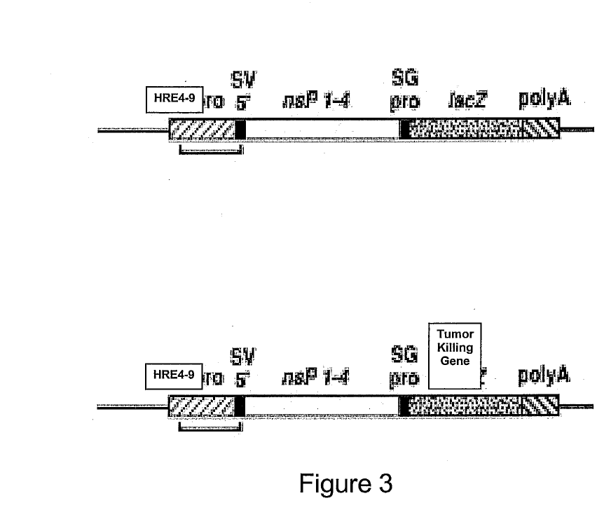

Truncated and mutant forms of bacterial toxins useful in this invention are shown in FIG. 3 of Kreitman R J & Pastan I Adv Drug Deliv Rev 31: 53-88 (1998) as described below. Amino acid 607 of PE and the remaining carboxyl terminal amino acids 608-613 are depicted. Pseudomonas exotoxin (PE) contains domains Ia (amino acids 1-252), I (amino acids 253-364), Ib (amino acids 365-399) and III (amino acids 400-613) are shown below. In PE4E; basic amino acids at positions 57, 246, 247 and 249 of PE are replaced by glutamate residues. In PE40, domain Ia has been removed from PE. In PE38, amino acids 365-380 have been removed from domain Ib of PE40. In PE38 KDEL, the carboxyl terminal amino acids REDLK (SEQ ID NO: 55) of PE38 have been replaced with KDEL (SEQ ID NO: 58). PE35 contains methionine followed by amino acids 281-364 and 381-613 of PE, and the only cysteine residue in PE35 is shown at position 287. Diphtheria toxin (DT) contains a methionine preceding amino acids 1-5 (GADDV) (SEQ ID NO: 60). DT contains an A chain (amino acids 1-193) and a B chain (amino acids 194-535). In DAB486, amino acids 486-535 of DT are removed, and in DT388 or DAB3g, amino acids 389-535 of DT are removed. All of these forms are useful in the claimed invention.

The 8H9 monoclonal antibody (MAb) is highly reactive with a cell surface glycoprotein expressed on human breast cancers, childhood sarcomas, and neuroblastomas but is not reactive with the cell surface of normal human tissues. This specific reactivity suggests that MAb 8H9 is useful for targeted cancer therapy. Two recombinant immunotoxins (ITs) using the single-chain Fv (scFv) of MAb 8H9 are particularly useful when fused to a truncated PE. The 8H9 (scFv) cDNA is fused to a DNA encoding a 38-kDa truncated form of Pseudomonas exotoxin (PE38) to generate the IT 8H9(scFv)-PE38. The fusion gene is expressed in Escherichia coli, and the IT is purified to near homogeneity from inclusion bodies. The purified IT showed specific cytotoxicity on nine different cancer cell lines derived from breast cancer, osteosarcoma, and neuroblastomas, known to react with MAb 81-19. The cytotoxic activity is inhibited by MAb 8H9, showing the cytotoxic activity is specific. The antitumor activity of 8H9(scFv)-PE38 evaluated in severe combined immunodeficient mice bearing MCF-7 breast cancers or OHS-M1 osteosarcomas showed specific dose-dependent antitumor activity at 0.075 and 0.15 mg/kg. A more stable disulfide-linked IT, 8H9(dsFv)-PE38, produced in high yield (16%) and showed cytotoxic and antitumor activities similar to those of 8H9(scFv)-PE38. 8H9(dsFv)-PE38 was given to two cynomolgus monkeys at doses of 0.1 and 0.2 mg/kg i.v. QOD×3 and was well tolerated. These results make 8H9(dsFv)-PE38 an excellent candidate for use in the present invention for treatment of breast cancers, osteosarcomas, and neuroblastomas (Brinkman et al., Proc. Natl. Acad. Sci. 88: 8616-8620 (1991); Onda M. et al., Cancer Res. 64, 1419-1424, (2004)).

The present invention is not confined to the latter tumor specific antibody. Any tumor specific antibody, fv, Fab fragment either single or double chain or tumor targeting ligand e.g., EGF, chemokine receptor ligand specific for any and all human tumors listed herein is useful in the present invention. The mesothelin tumor specific monoclonal antibody which has been fused to PE40 and shown broad anti-tumor activity is particularly preferred. Likewise, any other tumoricidal molecules or molecules that promote tumor killing, e.g., Panton-Valentine leukocidin (PVL) including but not limited to ricin, diptheria toxin, pertussus toxin either alone or coupled to a tumor specific targeting structure is useful in this invention. A targeting device and tumor toxin are conjugated as fusion proteins or biochemically crosslinked using well established technology.

A particularly preferable construct in the present invention is PE38-sc or dsFV or PE40-mesothelin incorporated into self replicating RNA alphavirus vectors as described below.

### Self Replicating RNA Vectors

As described above adeno-associated virus (AAV) has the characteristics of the long-term and efficient transgene expression in various cell types. However, disadvantages of the AAV include a restricted packaging capacity, inefficiency for large production, pre-existing immunity to human AAV vectors and integration into the host genome. While capable of delivering genes with high efficiency to a wide spectrum of non-dividing cells in vivo, adenoviruses induce a strong immune response of host cells directed against multiple viral structural epitopes which neutralize production of the desired heterologous protein.

Thus additional vectors are useful for transfection of tumor killing agents into the sickled erythroblasts and progenitors include the self-replicating RNA replicons (replicase nucleic acids) derived from alphavirus vectors, such as Sindbis virus, Semliki Forest virus, or Venezuelan equine encephalitis viruses. These viruses have a broad cell host range, readily transfect SS erythroblasts and rapidly replicate themselves (10⁹-10¹⁰ infectious particles/ml) as well as transgenic tumoricidal constructs in high titer. They are administered as either RNA or DNA, which is then transcribed into RNA replicons in transfected cells in vivo. Intracellular self replication of the native virus also induces apoptosis of the host cell. In the setting of the present invention, these self replicating vectors are both hemolytic and oncolytic. In contrast to AAV, preexisting and acquired immunity to alphaviruses in humans is rare as the virus is not integrated into the host genome.

The alphaviruses accomplish self-replication through the action of a polyprotein RNA replicase that is encoded within a single open reading frame. A single strand of RNA is directly translated by ribosomes (because of its positive polarity) producing the replicase polyprotein. This polyprotein is cleaved into four subunits that drive not only its own replication, but the replication of a structural protein that comprises the viral coat. Theoretically up to 200,000 copies of the RNAs and 100,000,000 molecules of heterologous proteins are made in a single cell.

### Structure of Alphaviruses

Alphaviruses, the major genus of the Togavirus family with 26 members commonly reside in many species such as mosquitoes, birds and rodents and other mammals. The alpha virus genome consists of approximately 12 kb as single-stranded RNA of positive polarity that is capped at the 5′ terminus and polyadenylated at the 3′ terminus. The alphavirus particle contains a single genomic RNA complex with 240 molecules of a basic capsid protein surrounded by a lipid bilayer containing E1 and E2 envelope glycoprotein heterodimers that trimerize producing a functional subunit and spike on the virus surface. The E1 glycoprotein is highly conserved among alphaviruses and is involved in cell attachment, membrane fusion and entry. The E2 glycoprotein contains the most potent epitopes eliciting neutralizing antibodies. The genomic RNA is encapsulated in a protein shell composed of a single protein subunit surrounded by a lipid bilayer consisting of two transmembrane glycoproteins. The 5′ portion of the alphavirus genome contains the genetic information encoding the nonstructural viral proteins required for transcription and replication of the viral RNA. The 3′ portion of the genome contains the genes encoding the viral structural proteins, such as the capsid protein and viral envelope glycoproteins.

### Replication Cycle of Alphaviruses

The alphavirus enters the cell by receptor-mediated endocytosis and after fusion of the virus with the endosomal membrane the viral nucleocapsid is released into the cytoplasm where translation of the non-structural viral protein (the replication complex) occurs. The viral structural proteins (capsid, envelope proteins) translated from 26S RNA (subgenomic RNA) are synthesized as polyproteins with the N-terminal capsid protein functioning as an auto-protease. The replication complex is required for initiation of viral RNA amplification. Four non-structural genes, nsP1-4, generate four polypeptides the replication complex after post-translational cleavage that function together in a replication complex required for the synthesis of the negative strand RNA from which an estimated 200,000 copies of RNA are made. RNA replication occurs via synthesis of a full length minus-strand intermediate that is used as a template for synthesis of additional genome length RNAs and for transcription of a plus-strand subgenomic RNA from an internal promoter. Thus the synthesis of minus, plus and subgenomic RNAs is regulated via proteolytic processing of non-structural polyprotein replicase components. Replication occurs entirely in the cytoplasm of the infected cells as an RNA molecule without a DNA intermediate. Assembly of RNA and capsid protein into nucleocapsids also occurs in the cytoplasm followed by transport to the plasma membrane where it acquires a lipid bilayer envelope with embedded viral glycoproteins. Simultaneously, the envelope proteins are processed through the Golgi apparatus and the endoplasmic reticulum to the plasma membrane, where they surround nucleocapsids. Finally mature virus particles are released by budding through the plasma membrane.

It is reported that SIN-targeted laminin receptors are expressed at much higher density on tumor cells than on normal cells (Tseng J et al., supra (2002)).

### Expression of Heterologous Genes

Alpha virus vectors have a large capacity to accommodate foreign gene with a size restriction of approximately 4 kb. It is possible to introduce at least 7 kb inserts meaning that several genes either under separate subgenomic promoters or Internal Ribosomal Entry Site (IRES) sequences can be inserted. In principle, three different types of vectors are constructed. All of these result in delivery of self replicating alphavirus vector into target cells as the native virus and/or with the expression of tumoricidal molecules being driven by a viral subgenomic promoter (Agapov E V et al., Proc Natl Acad Sci 95:12989-94 (1998); Frolov I et al., Proc Natl Acad Sci 93:11371-7 (1996); Frolov I et al., J. Virol. 68:1721-7 (1994); Frolov I et al., J. Virol. 73:3854-65 (1999); Invanova L et al., J. Virol. 73:1998-2005 (1999); Boorsma, M., et al., Nature Biotechnol., 18: 429-32 (2000); Lundstrom K., Gene Therapy 12: S92-S97 (2005); Yamanaka R., Int. J. Oncol. 24: 919-923, (2004); Leitner W W et al., Cancer Res. 60: 51-55 (2000); Tseng J et al., J Natl. Cancer Inst. 94:1790-802 (2002)) and optionally the highly efficient HRE.

I. Replication-Deficient Vectors:

Replication-deficient non-cytopathic alphavirus vectors such as SFV, Sindbis virus (SIN) and Venezuelan equine encephalitis virus (VEE) contain the viral nonstructural genes (nsP1-4) and the tumoricidal gene packaged into alphavirus particles. The generated recombinant alphavirus particles are capable of infection of host cells, but because no viral structural genes are accommodated, no further virus replication occurs. The obtained transgene expression is therefore of a transient nature.

II. Replication-Competent Viral Particles and DNA-Based Vectors:

In contrast to the suicide vectors described above, replication competent vectors contain the full-length alphavirus genome and an additional subgenomic promoter upstream of the transgene of interest. Infection of host cells with replication-competent particles leads to virus replication exclusively in the cytoplasm and expression of their genes is independent of host nuclear programs. The recombinant particles produced are infectious, capable of generating progeny virus in host cells that ultimately kill the host cell. Heterologous proteins such as the IgG domain of protein A, α- and β-human chorionic gonadotropin have been inserted into the viral envelope protein E2.

An additional strategy for inducing the expression of tumoricidal genes is to construct cDNAs of the alphavirus RNA genome wherein the tumoricidal genes are placed downstream from the promoter for a DNA dependent RNA polymerase used to transcribe a subgenomic RNA. To allow direct application of plasmid DNA, the SP6 RNA polymerase promoter is replaced by a CMV promoter which generates a long positive strand of RNA (replicon) and a tumoricidal protein which like the alphaviral genome itself is then capable of self replication. Transfection of SS erythroblasts with plasmid DNA results in high expression levels of the virus.

A DNA-based helper vector is also cotransfected to obtain recombinant particles. However, the titers are significantly lower than for RNA-based particles. In the SFV system, the replicase system 5′ and 3′ sequences needed for replication are intact and the structural genes are replaced by the tumoricidal polypeptide. The SFV system is suicidal therefore transmission of infectious particles from the cell targeted by the vector cannot occur. This avoids integration of the transgene into the chromosome or induction of tolerance. The risk of generating anti-vector immunity is low since no structural genes are encoded by the vector.

In an example of an RNA-based vector system encoding a recombinant tumoricidal gene or protein, the Sindbis virus is introduced into the cytoplasm of susceptible cells where it replicates and the virus genomic 49S RNA serves as the template for synthesis of a complementary negative strand by the virus-encoded replicase. The negative strand in turn serves as the template for additional genomic RNA and for an abundant internally initiated 26S subgenomic RNA. The nonstructural proteins (nsPs) are translated from the 59 two-thirds of the genomic RNA, while the structural proteins (sPs) are translated from the subgenomic 26S RNA that represents the 39 one-third of the genome. The nsP and sP genes are each expressed as polyproteins and are processed posttranslationally into the individual proteins.

Expression of heterologous proteins from alphavirus vectors is based on the same strategy as expression of the sPs of wild-type virus above and is initiated by transfection of in vitro-transcribed, self-replicating vector RNA (replicon) molecules. The region encoding the virus sPs is replaced with a heterologous sequence or gene of interest, and the viral nsP-encoding region and all sequences required in cis for replication and packaging are maintained. Heterologous sequences are synthesized as highly abundant subgenomic mRNA molecules, which in turn serve as the translational template for the heterologous gene. Infectious vector particles have been generated by cotransfection and trans complementation of vector RNA replicons with an in vitro-transcribed defective helper (DH) RNA. The DH RNA contains the genes encoding the virus sPs and all of the sequences required in cis for replication but is deleted in the viral nsP genes and the virus packaging sequence core. Thus, replication of the DH RNA and expression of high levels of the sPs occur in the presence of vector-supplied nsPs and result in the production of particles containing vector genomes.

In the present invention, the replication-competent SinRep/LacZ or pSINrep5 vectors in native form or containing a CMV promoter and the HRE enhancer are operatively linked to a nucleic acids encoding an FMG Measles F protein gene (fusogenic membrane glycoprotein, MGF) described earlier that facilitates the dispersion and distribution of viral gene products within the tumor mass or a tumoricidal polypeptide such as the sc8H9 (Fv)-PE38. The FMG or tumoricidal protein is inserted into the Sindbis RNA and DNA expression vectors and defective helpers as described below. The HRE is optionally inserted just downstream of the subgenomic promoter (Dubensky et al., J. Virology 70: 508-516 (1996)).

In an additional embodiment the hyperfusogenic mutant of the gibbon ape leukemia virus envelope glycoprotein (BALV.fus) is expressed in Sindbis virus replicon containing infectious particles in high titler by cotransfecting vector and helper RNAs into baby hamster kidney (BHK-21) cells. Packaged GALV.fus expressing Sindbis vectors can be used to transfect SS RBCs, SS erythroid precursors and erythroleukemia cells. The FMG Measles F protein gene (fusogenic membrane glycoprotein, MGF) and any other effective fusogenic particle is similarly integrated into the Sindbis virus replicon.

### Methods

Sindbis virus plasmid DNA and RNA replicon expression vectors contained viral nt 1 to 7643, pKSII1 polylinker, viral nt 11664 to 11703, and a 25-mer synthetic poly(A) tail and are constructed from the pRSINg, pDLTRSINg, and pDCMVSINg plasmids. The RNA expression vector contains the SP6 promoter at its 59 end, and the DNA expression vectors contained either the MoMLV LTR, SV40, or CMV IE promoter at their 59 ends and the bovine growth hormone transcription termination/polyadenylation signal at their 39 ends. The PCR amplicon product obtained with primer pair SIN3144F and SIN7643R (Table 1, Dubensky et al., J. Virology 70: 508-516 (1996)) is used to construct a portion of the expression vectors which includes nt1 to 7643. A unique XhoI site is introduced into the 59 end of primer SIN7643R to facilitate insertion of the amplicon between the SfiI site at Sindbis virus nt5122 and the XhoI site in the pKSII1 polylinker. The primer pair SIN11644F and SIN11703R (Table 1 Dubensky et al., J. Virology 70: 508-516 (1996)) PCR amplicon product is used to assemble the vector 39 end between unique NotI and SacI sites at the 39 end of the pKSII1 polylinker. For insertion into DNA expression plasmids, the 39-end SacI site of the Sindbis virus vector and the unique XbaI site of pcDNA3 are digested and blunted with T4 DNA polymerase, and the fragments are ligated.

The FMG gene is inserted into the polylinker of the DNA and in vitro-transcribed RNA-based expression vectors. These constructions are designated pRSIN-FMG (in vitro-transcribed RNA expression vectors) and pDLTRSIN-FMG and pDCMVSIN-FMG (DNA expression vectors). Linearization of pRSIN-FMG for in vitro transcription is done with SacI or PmeI, respectively.

In other constructions, the FMG is inserted between the synthetic A25 tract and the transcription termination/polyadenylation signal of the pDLTRSIN-vector. The FMG sequence, with SacI sites at each end, is generated by PCR. Correct- and reverse-sense HDV insertions are verified by sequence analysis; these constructions are designated pDLTRSIN-lucFMG.

Sindbis vector plasmid Sinrep5LacZ encoding beta-galactosidase and helper plasmid DH-BB are prepared as described by Bredbeek P et al., J. Virol. 67: 6439-6446 (1993). Plasmid Sinrep5GALV.fus is made by replacing the β-galactosidase gene in Sinrep5LacZ with the GALV.fus gene. The GALV.fus gene is amplified from plasmid FB.CD40L.X.Galv.fus which is derived from FBMoSALF containing GaLV R- by PCR amplification (Fielding A L et al., Blood 91: 1802-1809 (1998). Primers used for PCR are GALV.fus.Xba (SEQ ID NO: 61) (5′-CTAGTCTAGAATGGTATTGCTGCCTGGGTCC-3′) and GALV.fus.Sph (SEQ ID NO: 62) (5′-ATATCGGCATGCACATGCACTTATCC-TATCATTG-3′). The PCR product is digested with Xba I and Sph I restriction enzymes and subcloned into the Sinrep5LacZ vector. The insert is verified by DNA sequencing. The plasmids were prepared with a QIAfilter Plasmid Midi kit (Qiagen) for further use.

Packaging of Sindbis vector into infectious virus particles is described by Bredbeek supra (1993). Briefly, vector and helper plasmid DNAs are linearized by restriction enzyme Xho I, and then used to perform an in vitro transcription. The transcription reaction uses 10 μl 5× buffer, 5 μl 10 mM DTT, 10 μl rNTP mixture (2.5 mM each, Boehringer), 2.5 μl 10 mM M⁷G(5′)ppp(5′)G RNA CAP analog (New England Biolabs), 2 μg DNA, 1 μl RNaseOUT (40 u, GIBCO), 2.5 μl SP6 polymerase (15 u/ul, GIBCO) and RNase-free water is added to 50 μl in total and incubated on a water bath at 41° C. for 1 h. RNA transcripts are loaded onto 1% agarose denaturing gels to evaluate RNA, concentration and integrity, and quantitated by measuring OD260 Vector and helper RNAs are co-transfected into BHK-21 cells and incubated at 37° C. Then, 36-48 h later, the supernatants are harvested, filtered with a 0.45-μm filter and stored at −80° C. for future use. For virus concentration, 10 ml virus-containing media are ultracentrifuged in a Beckman 41 rotor at 30K rpm for 1 h. The supernatant is discarded and the virus pellet is resuspended in 100 μl phosphate-buffered saline (PBS) or serum-free Dulbecco's modified Eagle's medium (DMEM).

The vectors are initially transfected into BHK cells, viral particles isolated and used to infect sickled erythroblasts ex vivo. The infected SS erythroblasts are administered in vivo and are trapped in the hypoxemic microcirculation of tumors whereupon synthesis of the Sindbis virus is activated via the HRE. The virus replicates rapidly in high titer (10⁹-10¹⁰ infectious particles/ml) and induces apoptosis of the SS erythroblast with shedding of the virus in large numbers into the surrounding tumor tissue. The virus infects tumor cells via binding of its laminin receptor to laminin expressed on tumor cells. The viral-infected tumor cells are lysed by the virus. Cell to cell transfer is facilitated by co-integration of FMG gene or other fusogenic molecule or VP22 protein into the SINrep5 vector with a tumoricidal polypeptide into the viral genome (Cheng, W et al J. Virol. 75: 2368-2376 (2001)).

sc8H9 (Fv)-PE38 and any of its variants are the preferred tumoricidal toxin for use in the alphaviral and other viral constructs described herein. Other tumor toxins are useful as well as other tumor targeting agents. PE38 is the truncated form of Pseudomonas exotoxin A and psc8H9 is the expression vector for which encodes the PE38 fused to single or double chain tumor specific Fv specific for adenocarcinomas as described in FIG. 1 of Onda et al., Cancer Res. 64: 1419-24 (2004)). DNA fragments encoding sc8H9 (Fv)-PE38 are isolated by digesting psc8H9 (Fv)-PE38 with NdeI and EcoRI restriction enzymes. These isolated DNA fragments are further cloned into the corresponding XbaI and PmeI sites of the SINrep5 or SinRep/2PSG vectors to generate SINrep-sc8H9 (Fv)-PE3 (Onda et al., supra (2004)).

Any alphavirus vector is useful whether it is replication competent or incompetent, cytopathic or non-cytopathic for host cells. Replication competent and cytopathic native alphavirus particles containing an inducible promoter and enhancer such as HRE are transfected into sickled RBCs, their precursor erythroblasts or erythroleukemia cells. When these erythroblasts are deposited in the tumor vasculature, the virus-infected cell bursts shedding alphavirus particles into the surrounding media. The process of hemolysis may be facilitated by the exposure of the virus-infected SS erythrocytes, SS precursors or erythroleukemia cells to light irradiation under conditions (cells: 10⁶ ml⁻¹; time: 2 min from 10 mm distance; source: ‘black-light’ delivering 10 μm⁻²; emission light spectra in the region 320-450 nm, with a maximum at 380 nm. These conditions induce photohemolysis t½ of 10-60 minutes after light exposure allowing for parenteral administration of virus-infected cells and their localization in tumor neovasculature where hemolysis takes place with viral shedding into the tumor milieu. Because of their specificity for laminin receptors on tumor cells, the Sindbis virus particles with fusogenic particles selectively infect and lyse surrounding tumor cells. While other alphaviruses are useful, the native Sindbis virus is preferred.

Replication incompetent and non-cytopathic Sindbis virus vectors in which a tumor toxin or a viral fusogenic gene such as the FMG (optionally under control of an HRE or other inducible enhancer/promoter) is substituted for viral structural genes are also useful. In this system, the sickled erythroblast once deposited in the hypoxemic tumor microvasculature expresses and secretes a tumoricidal toxin such as sc8H9(Fv)-PE38. The replication competent vector that lyses the carrier SS cell while producing the tumoricidal proteins or fusogenic genes is preferred. However, a replication incompetent vector that does not lyse the sickled erythroblasts is also useful. The SS erythroblast continues to produce and secrete the tumoricidal proteins in high titer that selectively attack and kill surrounding tumor cells. Specific methods for producing replication competent/incompetent or cytopathic/non-cytopathic alphavirus vectors are given in Example 4.

Any biologically acceptable tumoricidal molecule is useful when integrated into the viral constructs listed herein. Particularly relevant molecules that are integrated into the cloning site of the self-replicating viruses are staphylococcal alpha hemolysin, listeria hemolysin and Panton Valentine Leukocidin (PVL). The former two released from SS erythroblasts in tumor sites are capable of inducing hemolysis of the parent SS erythroblasts and tumor oncolysis. Likewise, Panton Valentine Leukocidin (PVL) released into tumor tissue from SS erythroblasts attracts and induces apoptosis of polymorphonuclear leukocytes leading to necrosis of tumor cells.

| The structure of staphylococcal alpha hemolysin is | |
|---|---|
| described by Song, L et al Science 274: 1859-1866 (1996). | |
| [SEQ ID NO: 63] | |
| Alpha hemolysin | |
| 1 | mktrivssvt ttlllgsilm npvagaadsd iniktgttdi gsnttvktgd |
| | lvtydkengm |
| 61 | hkkvfysfid dknhnkkllv irtkgtiagq yrvyseegan ksglawpsaf |
| | kvqlqlpdne |
| 121 | vaqisdyypr nsidtkeyms tltygfngnv tgddtgkigg liganvsigh |
| | tlkyvqpdfk |
| 181 | tilesptdkk vgwkvifnnm vnqnwgpydr dswnpvygnq lfmktrngsm |
| | kaadnfldpn |
| 241 | kassllssgf spdfatvitm drkaskqqtn idviyervrd dyqlhwtstn |
| | wkgtntkdkw |
| 301 | tdrsseryki dwekeemtn |
| The structure of Listeria hemolysin is delineated | |
| by Nelson, KE et al., Nucleic Acids Res. 32: | |
| 2386-2395 (2004). | |
| [SEQ ID NO: 64] | |
| Listeria hemolysin | |
| 1 | mtikkeradi llveqglfet rekakraima givyrkeerv dkpgekipid |
| | elqvkgkqm |
| 61 | pyvsrgglkl ekalqvfdfe vkdklmldig astggftdca lqngarhsya |
| | ldvgynqlaw |
| 121 | klrnddrvtv mertnfrhvt padfseglad fatidvsfis lklilpvlrt |
| | vlvtggdvmt |
| 181 | likpqfeagr eqvgkkgiir dpavhesvve hivqfaldng ydlmgldfsp |
| | itggegnief |
| 241 | ahlkwtgge tgtnhlepna iaklitkaht kldk |
| The structure of Panton Valentine Leukocidin is | |
| delineated by Nakagawa S et al., Biochem. Biophys. | |
| Res. Commun. 328, 995-1002 (2005).). | |
| [SEQ ID NO: 65] | |
| Panton Valentine Leukocidin | |
| 1 | mvkkrllaat lslgiitpia tsfheskadn nienigdgae vvkrtedtss |
| | dkwgvtqniq |
| 61 | fdtvkdkkyn kdalilkmqg finskttyyn ykntdhikam rwpfqynigl |
| | ktndpnvdli |
| 121 | nylpknkids vnvsqtlgyn iggnfnsgps tggngsfnys ktisynqqny |
| | isevehqnsk |
| 181 | svqwg ikans fitslgkmsg hdpnlfvgyk pysqnprdyf vpdnelpplv |
| | hsgfnpsfia |
| 241 | tvshekgsgd tsefeitygr nmdvthatrr tthygnsyle gsrihnafvn |
| | rnytvkyevn |
| 301 | wktheikvkg hn |

A typical pharmaceutical toxin composition for intravenous administration includes about 0.1-10 mg per patient per day. Dosages from 0.1 up to about 100 mg per patient per day may be used particularly if the agent is administered to a secluded site and into the circulatory or lymph system such as into a body cavity or into a lumen of an organ. This amount of toxin can be readily generated in approximately 10-100 cc of sickled erythrocytes once activated under hypoxemic conditions. Actual methods for preparing administrable compositions will be known or apparent to those skilled in the art are described in more detail in such publications as Remington's Pharmaceutical Science, 10th ed. Mack Publishing Company, Easton, Pa. (1995).

siRNA

RNA interference (RNAi) is a highly conserved gene silencing mechanism that uses double-stranded RNA (dsRNA) as a signal to trigger the degradation of homologous mRNA. The mediators of sequence-specific mRNA degradation are 21- to 23-nt small interfering RNAs (siRNAs) generated by ribonuclease III cleavage from longer dsRNAs. A short (usually 21-nt) double-strand of RNA (dsRNA) with 2-nt 3′ overhangs either end. Twenty-one-nucleotide siRNA duplexes trigger specific gene silencing in mammalian somatic cells without activation of the nonspecific interferon response.

Transfection of an exogenous siRNA is enhanced by introduction of a loop between the two strands, thus producing a single transcript, which can be processed into a functional siRNA. Such transcription cassettes typically use an RNA polymerase III promoter (e.g. U6 or H1), which usually direct the transcription of small nuclear RNAs (snRNAs) (U6 is involved in gene splicing; H1 is the RNase component of human RNase P). The resulting siRNA transcript is then processed by Dicer.

In one embodiment of the present invention, sickled erythroblasts are transduced in vitro with vectors encoding siRNAs that induce or contribute to tumoricidal activity. The therapy is applicable to carcinomas, sarcomas, gliomas, melanomas and lymphomas/leukemias. The spectrum of vectors that have been used effectively as vehicles for siRNAs transfection in experimental tumor models to date is given in Table 6. The present invention contemplates the use of any of these vectors optionally under control of the HRE or other suitable promoter as useful. Typically, the sickled erythroblasts are transfected in vitro with the siRNA using siRNA expression vectors or PCR products. Synthetic siRNAs chemically synthesized or in vitro transcribed siRNAs can be transfected into cells or injected into mice. A self-replicating cytopathic alphavirus vector is preferred such as the SINrep5 which expresses Sindbis virus structural proteins that recognize laminin receptors on tumor cells. The siRNA of choice is integrated into the cloning region of these viruses or cotransfected with them.

When administered parenterally, to tumor bearing hosts, the transfected SS erythroblasts are capable of lysing the erythroblast and shedding the vector, containing the siRNA to infect surrounding tumor cell selectively. Alphaviruses with self replicating replicons such as the Sindbis and SFV that can lyse the SS erythroblast carrier and infect adjacent tumor cells are preferred but other tumor specific viruses shown in Tables 1A and 1B such as dll150 and those avid for HIF in tumor cells are also useful. Optionally, the VP22 or other peptides that promote cell to cell transfer are cointegrated into the alphavirus (pRep5) vector together with the siRNA. DNA fragments encoding VP22 are isolated by digesting pcDNA3-VP22, respectively, with XbaI and PmeI restriction enzymes. These isolated DNA fragments are further cloned into the corresponding XbaI and PmeI sites of the SINrep5 vector to generate SINrep5-siRNA-VP22 constructs.

An adenovirus is one of the most well-known viral vectors for gene delivery. Intratumoral injection of an adenovirus encoding the hypoxia-inducible factor-1 (HIF-1)-targeted siRNA had a significant effect on tumor growth when combined with ionizing radiation (Zhang et al., Cancer Res. 64:8139-42 (2004)). The very same construct is used to infect SS erythroblasts that are lysed by the virus leading to viral shedding into surrounding tumor tissue. The virus selectively infects hypoxic tumor cells expressing HIF-1 and induces apoptosis via siRNA targeting of HIF-1. Oncolytic viruses similarly transfected with a siRNA targeting a gene overexpressed in tumor cells can lyse the tumor cell via siRNA inactivation of a key genetic function or by endogenous self-replication.

Candidate target genes for RNAi-mediated knockdown are selected from several key oncogenes, antiapoptotic genes or tumor promoting genes, including growth and angiogenic factors or their receptors. As a matter of course cancer-specific genes selectively overexpressed, mutated or translocated are chosen. Initial in vitro studies have demonstrated effective silencing of a wide variety of mutated oncogenes such as K-Ras, mutated p53, Her2/neu and bcr-abl. siRNA software is available for design of effective siRNA sequences (Takeshita F., Cancer Sci 97: 689±696 (2006)).

TABLE 6
Delivery of small interfering RNA (siRNA) in cancer models

| | | | Implanted Site | |
|---|---|---|---|---|
| Carriers | Routes | Type of cancer (cell line) | (target organ) | Target gene |
| Naked siRNA | i.p., i.v., s.c., i.t. | Fibrosarcoma | s.c. | VEGF |
| | | (JT8) | | |
| Naked siRNA + gemcitabine | i.v. | Pancreatic adenocarcinoma | Orthotopic pancreas | FAK |
| | | (PANC1, MIAPaCa2, BxPC3) | | |
| Naked siRNA | i.v. | Pancreatic adenocarcinoma (BxPC3) | s.c., | CEACAM6 |
| | | | Orthotopic pancreas | |
| Naked siRNA | i.v. | Pancreatic adenocarcinoma | s.c., | EphA2 |
| | | (PANC1, MIAPaCa2, BxPC3, | Orthotopic pancreas, | |
| | | Capan2) | liver metastasis | |
| Naked siRNA + gemcitabine | i.v. | Pancreatic adenocarcinoma | s.c., | RRM2 |
| | | (PANC1, MIAPaCa2, BxPC3, | Liver metastasis | |
| | | Capan2) | | |
| Naked siRNA | i.v. | Breast cancer | Lung metastasis | CXCR4 |
| | | (MDA-MB-231) | | |
| Liposome | i.p. | Colon cancer | s.c., i.p. | β-Catenin |
| | | (HTC116) | | |
| Liposome | i.v. | Liver metastatic spleen cancer (A549) | Liver | bcl-2 |
| Liposome | i.t. | Bladder cancer (UM- | Bladder | PLK-1 |
| | | UC-3-LUC) | | |
| Liposome | i.t. | Pancreatic carcinoma | s.c. | Somatostatin |
| | | (Capan-1) | | |
| CCLA (NeoPhectin-AT) | i.v. | Prostate cancer (PC- | s.c. | Raf-1 |
| | | 3) | | |
| CCLA | i.v. | Breast cancer | s.c. | c-raf |
| | | (MDA-MB-231) | | |
| shRNA plasmid + pegylated | i.v. | Glioma | Brain | EGFR |
| immunoliposome | | (U87) | | |
| PEI | i.p. | Ovarian carcinoma cells | s.c. | HER-2 |
| | | (SKOV-3) | | |
| shRNA plasmid + PEI | i.t. | Ewing's sarcoma | s.c. | VEGF |
| | | (TC71) | | |
| Adenovirus vector | i.t. | Cervical adenocarcinoma, colon | s.c. | HIF-1α |
| | | cancer (HeLa, HTC116) | | |
| Adenovirus vector | i.t. | Lung cancer | s.c. | Skp-2 |
| | | (ACC-LC-172) | | |
| shRNA plasmid | i.t. | Glioblastoma | Brain | MMP-9 + |
| | | (SNB19) | | cathepsin B |
| shRNA plasmid | i.t. | Glioma | Brain | Cathepsin B. uPA |
| | | (SNB19) | | |
| shRNA plasmid + ATA | i.v. | Cervical adenocarcinoma, lung | s.c. | PLK1 |
| | | cancer (HeLa S3, A549) | | |
| PEI-PEG-RGD | i.v. | Neuroblastoma | s.c. | VEGF-R2 |
| | | (N2A) | | |
| CDP-AD-PEG-transferrin | i.v. | Ewing's sarcoma | Multiple organ metastasis | EWS-FLII |
| | | (TC71) | | |
| HVJ envelope vector + cisplatin | i.t. | Cervical adenocarcinoma (HeLa) | Intradermally | Rad51 |
| ErbB2-protamine fusion protein | i.t., i.v. | Melanoma | s.c. | c-myc |
| | | (B16) | | MDM2 |
| | | | | VEGF |
| | | | | (mix) |
| Atelocollagen | i.t. | Prostate cancer (PC- | s.c. | VEGF |
| | | 3) | | |
| Atelocollagen | i.t. | Germ-cell tumor | Testis | FGF-4 |
| | | (NEC8) | | |
| Atelocollagen | i.v. | Prostate cancer (PC- | Bone metastasis | EZH2, p110α |
| | | 3M-Luc) | | |

AD-PEG, adamantane-PEG5000;
ATA, aurintricarboxylic acid, nuclease inhibitor;
CCLA, cationic cardiolipin analog-based liposome;
CDP, cyclodextrin-containing polycations;
i.p., intraperitoneal;
i.t., intratumoral;
S i.v., intravenous;
PEG, polyethylene glycol;
PEI, polyethylenimine;
RGD, Arg-Gly-Asp;
s.c., subcutaneous

The preparation of siRNA duplexes specific for and capable of inactivating the target genes given in Table 3 are described in Example 5.

SS Erythrocytes, SS Erythroblasts and Erythroleukemia Cells Transduced with Multi-Drug Resistant Genes

The multi-drug resistance gene, MDR1 encodes an ATP-dependent plasma membrane efflux pump, P-glycoprotein (P-gp). These transporters and several others including the ABG transporters are present in SS hematopoietic progenitors and erythroleukemia cells. In contrast to the products of other drug resistance genes, the P-gp extrudes a broad range of hydrophobic drugs from cells, including vinca alkaloids, anthracyclines, epipodophyllotoxins, colchicine, actinomycin D, taxol and taxotere. Transfer and expression of human MDR1 in marrow cells has been used to protect hematopoietic cells against myelotoxic drugs. Transgenic mice expressing the human MDR1 gene and normal mice transplanted with marrow from MDR1 expressing transgenic mice did not develop leukopenia after treatment with cytotoxic drugs presumably due to chemoprotection of transduced cells by MDR1 gene expression. MDR1 has been combined with other drug resistance genes to broaden the spectrum of drugs for combinatorial chemoprotection of transduced human stem cells: Mutants of P-gp have been used to tailor drug resistance profiles and are useful in this invention. For instance, the wild-type version of human MDR1 (containing Gly at position 185) confers preferential resistance against vinblastine while the mutant with Val at position 185 confers resistance to colchicine.

In the claimed invention, nucleic acids encoding the MDR1 optionally placed under the transcriptional control of the HRE enhancer are transfected ex vivo into sickle erythroblasts, hematopoeitic stem cells or nucleated erythroleukemia cells stably transfected with BCAM/Lu or other molecule(s) which bind to tumor neovasculature. These cells are then co-cultured with tumor cytotoxic drugs preferably in prodrug form. Loading of the cells with drug is accomplished by osmotic diffusion or electroporation and other methods well established in the art. These cells are then infused into the tumor bearing host. The transduced erythroblasts or erythroleukemia cells deposit in the hypoxemic tumor microvasuclature wherein the MDR1 gene is activated leading to efflux of the resident cytotoxic drug or prodrug into surrounding tumor tissue. The cytotoxic drug kills tumor cells directly. The invention is not confined to the MDR gene. ABG group of transporters and any other drug transporters in SS hematopoietic precursors are relevant and useful in this invention for the transport of tumoricidal drugs and toxins.

The expression of drug metabolizing cytochrome P450s (CYPs) notably 1A, 1B, 2C, 3A, 2D subfamily members has been identified in a wide range of human cancers. Individual tumor types have distinct P450 profiles as studied by detection of P450 activity, identification of immunoreactive CYP protein and detection of CYP mRNA. Selected P450s, especially CYP1B1, are overexpressed in tumours including cancers of the lung, breast, liver, gastrointestinal tract, prostate, bladder. Several prodrug anti-tumour agents have been identified as P450 substrates. Those in clinical use include prodrug alkylating agents cyclophosphamide, ifosphamide, dacarbazine, procarbazine, Tegafur, a prodrug fluoropyrimidine, methoxymorphylinodoxorubicin, a metabolically activated anthracycline, as well as flutamide and tamoxifen, two non-steroidal hormone receptor antagonists that are significantly more active following CYP-hydroxylation. New agents selectively dependent on tumor CYP activation include 2-(4-aminophenyl) benzothiazoles exclusively in CYP1A1 inducible tumors. Some CYPs operate most effectively under hypoxemic conditions. Indeed, bioreductive prodrugs such as the indolequinone AQ4N (a CYP3A substrate) and MUP 98176 are activated to cytotoxic metabolites specifically in hypoxic tumor regions after bioreduction.

In the present invention, drug resistant SS hematopoietic stem cells are produced by sustained exposure to prodrugs ex vivo. These cells are then administered to tumor bearing hosts and localize in the tumor vasculature where the bioreductive prodrug is transported out of the cell and taken up by surrounding tumor cells. Tumor cells overexpress oxyreductase systems cytochrome P450 enzymes and/or its congeners oxidize prodrugs to their reduced and active state resulting in oncolysis. Several of these active metabolites are significantly more cytotoxic under hypoxemic conditions within tumor cells.

For example, an SS cell progenitor or erythroleukemia cell stably transfected with an adhesion receptor such as BCAM/Lu whose cognate ligand laminin-α5 is expressed on tumor neovasculature is rendered drug resistant by exposure to chemotherapy for period known to induce resistance to a particular drug. At the end of this time frame, the drug-resistant cells are known to expel drug at a rate 4-10 fold faster than control non-drug resistant cells. Specifically, the K562 erythroleukemic cell line stably transfected with BCAM/Lu or other molecule(s) that bind to tumor neovasculature is rendered drug resistant after continuous exposure to small doses of Adriamycin for 120 hours after which Adriamycin is completely expelled from the cell over a period of 30 minutes (Yanovich et al., Cancer Res. 44, 4499-4505 (1989)). In the present invention, erythroleukemia cells or hematopoietic precursors stably transfected with BCAM/Lu or SS progenitor cells are exposed to various forms of chemotherapy and the optimal time course for development of drug resistance and release following discontinuation of drug is determined. After induction of drug resistance, these cells (10⁸-10¹²) are infused into tumor-bearing hosts where they deposit and release their drug directly into the tumor milieu. All forms of chemotherapy for which drug transport systems exist the erythroleukemia or SS progenitor population are eligible for this treatment including but not limited to the MDR and ABG transporters. Drug resistant erythroleukemia cells or erythroblasts are preferred drug carriers because the chemotherapeutic that is expelled from the cell does minimal damage to the cell itself.

Optionally, ex vivo exposure of both drug resistant and non-drug resistant cells (incubated with a tumoricidal drug for 8-120 hours) to light radiation (200-900 nM) that induces a hemolysis t½ of 20-60 minutes is useful to ensure release of the drug from the carrier SS cells, SS progenitors or erythroleukemia cells once they have deposited in the tumor vasculature. In this way chemotherapy can be specifically targeted to and concentrated in the tumor.

Likewise, nucleic acids encoding monoclonal antibodies specific for epitopes expressed on tumor cells, tumor parenchyma or tumor vasculature can be transfected into the SS progenitor or erythroleukemia cells using recombinant vectors well established in the art. An example of one such monoclonal antibody is Avastin specific for VEGF receptors on tumor endothelium. SS cells or erythroleukemia cells localized in the tumor vasculature release the VEGF-specific monoclonal antibodies into the tumor milieu. The tumor neovasculature is within easy reach of the recombinant antibodies with epitopes expressed on tumor endothelium and endothelial matrix such as VEGF and laminin-α5. In this way, anti-angiogenic therapy such anti-VEGF is concentrated at the site of its cognate ligand in the tumor neovasculature, produces an increase in the therapeutic index of the drug and reduction in its systemic side effects.

### Photolysis of Viral-Transduced Ss Cells, Ss Progenitor Cells and Erythroleukemia Cells

SS erythrocytes produce an abundance of hemoglobin degradation products rendering them photosensitive. Non-enzymatic heme-iron degradation is initiated by autooxidation of hemoglobin S and randomly attacks carbon-methene bridges of the heme moiety tetrapyrrole rings. This results in a 4-10 fold increase in fluorescent heme degradation products (FHDP) such as hemichromes and protoporphyrin IX, release of free iron and generation of 2 fold higher amounts of reactive oxygen species (ROS) than normal RBCs. Membrane-bound hemichromes produced in this process along with earlier bound hemoglobin S are targeted by activated O₂, superoxide and OH⁻ radicals resulting in membrane injury and cell lysis.

Protoporphyrins produced during the intermediate metabolism of heme are among the most effective RBC photosensitizers. In SS cells, their progenitors and erythroleukemia cells the intracellular concentration in RBCs increases after administration of the precursor 5-aminolevulinic acid. Other RBC photosensitizers effective in this invention include but are not limited to furocoumarins, xanthene dyes, α-alkylamino-2-arylquinolinemethanol antimalarial compounds, chlorpromazine, griseofulvin, carprofen, phthalo-cyanine sulphonates, sulphonated chloro-aluminium phthalocyanine (AIPcS), chlorin e₆ (Chl-e₆), HY and the mono-sodium salt (HY-Na), haematoporphyrin derivative (HPD), Photofrin® (PF), haematoporphyrin (HP), and benzo-porphyrin derivative monoacid ring A (BPD-MA), protoporphyrin IX. In the claimed invention, mature SS cells, SS progenitors and erythroleukemia cells (stably transduced with BCAM/Lu) containing increased amounts of naturally produced or exogenously induced photosensitizers such as deoxyhemoglobin, denatured hemoglobin and protoporphyrin IX are infected with oncolytic viruses as described herein with virus yields of 10⁵-10¹⁰ p.f.u./ml 48 hours post infection. These cells are then exposed to visible, ultraviolet or laser light in a range or 200-900 nM for 2-60 minutes which induces a hemolysis t½ of 30-60 minutes (Grossheimer L I Photosensitization of Red Blood Cell Hemolysis: A Brief Review http://www.photobiology.com/reviews/5/index.htm (1998); Bilgin M D et al., Photochem Photobiol. 72:121-7 (2000); Grossweiner L I et al., Lasers Med Sci 13: 42-54 (1998); Fernandez J M et al., J. Photochem. Photobiol. B: Biology 37: 131-140 (1997)). The latter references and their references cited are incorporated by reference in entirety. The cells (10⁵-10¹²) are administered parenterally and localize in the tumor neovasculature where photohemolysis takes place with shedding of oncolytic virus into the surrounding tumor milieu.

Various photosensitizing agents listed above are incubated with mature SS cells, SS progenitors and erythroleukemia cells transduced with oncolytic virus ex vivo before exposure to a visible light wave source for a duration that ensures t½ hemolysis in 30-60 minutes after administration to tumor bearing hosts. In a typical regimen using Lutetium (III) texaphryrin (PCI-0123; Lu-Tex) as photosensitizer, SS cell isolated from fresh human blood from a SS homozygous patient with sickle cell anemia and diluted in pH 7.4 phosphate buffered saline (PBS) to give a light-scattering OD=2.0 at 750 nm. The cells are incubated with Lu-Tex for 90 min at 37° C. in PBS. The Lu-Tex is pre-treated by bath sonication for 30 min at 25° C. The final RBC concentration is 5.0×10⁷ for human cells. One set of irradiations is made with the unbound Lu-Tex in the external medium. In another set of irradiations the cells are centrifuged and resuspended two times to remove the unbound Lu-Tex. Spectral measurements showed that >97% of the initially bound Lu-Tex remains bound to the RBC. The cells are irradiated in a 2 cm×2 cm cylindrical cuvette with oxygen bubbling and slow stirring while the transmission at 633 nm is monitored with a 1 mW He—Ne laser. The irradiation source is a Quantum Devices Model QBMEDXM-728 multi-element LED (730 nm maximum, 35 nm FWHM) located 3 cm from the irradiation cuvette. The on-axis incident fluence rate measured with a Newport Model 835 power meter is 63 mW cm⁻² (Bilgin et al., supra (2000)). Hemolysis is negligible during the irradiations.

One of skill recognizes that this is a model for the Lu-Tex photosensitization and that other photosensitizers are effective in initiating SS cell hemolysis. Conditions for maximum operability of these agents in the present invention may vary with each photosensitizer but not require undue experimentation. Any photosensitizing agent is useful in this invention including but not limited to 5-aminolevulinic acid, protoporphyrin IX, Texaphrin, furocoumarins, xanthene dyes, α-alkylamino-2-arylquinolinemethanol antimalarial compounds, chlorpromazine, griseofulvin, carprofen, phthalo-cyanine sulphonates, sulphonated chloro-aluminium phthalocyanine (AIPcS), chlorin e₆ (Chl-e₆), HY and the mono-sodium salt (HY-Na), haematoporphyrin derivative (HPD), Photofrin® (PF); haematoporphyrins (HP), and benzo-porphyrins.

In one embodiment, erythroleukemia cells stably transfected with BCAM/Lu and infected with oncolytic virus as given herein are grown in Dulbecco's modified Eagle's medium supplemented with 15% fetal calf serum (Gibco) in a humidified incubator enriched with 10% CO₂ at 37° C. supplemented with cold 5-amino levulinic acid (5-ALA) 5×10⁻⁴M and [4⁻¹⁴C]ALA (0.1/μCi ml⁻¹). The cells are subdivided twice a week by resuspension in fresh medium at a concentration of ˜5×10⁵ cells ml⁻¹. After 8 days of culture, cells (10⁸) are harvested, washed twice in PBS (0.1 M, pH=7.2). The resuspended cells (10⁶ ml⁻¹) are irradiated for 2 min from 10 mm distance, using a ‘black-light’ source, delivering 10 μm⁻². The emission spectra of the light are in the region 320-450 nm, with a maximum at 380 nm (Malik Z et al., Br. J. Cancer 56: 589-595 (1987). At the end of light exposure the cells are immediately collected and injected into tumor bearing hosts. The administered erythroleukemia cells localize to the tumor neovasculature within 10 minutes after delivery, undergo photohemolysis 20 minutes later and shed their oncolytic viral contents into the tumor milieu. The light exposure may also be delivered to the host from an exogenous source after the administrated erythroleukemic cells have localized in tumors (usually 5-30 minutes after intravenous injection/infusion).

In an additional embodiment, protoporphyrin IX accumulation in the same erythroleukemia cells and specific cell lysis induced by exposure to 1 mM delta-aminolevulinic acid (ALA) for 2-5 hours is increased significantly by inclusion with ALA of 1,10-phenanthroline (0.75 mM), a tetrapyrrole biosynthesis modulator during the incubation in a method described by Rebeiz N et al Photochem Photobiol 44: 679-687 (1986).

In another embodiment, tumor-localizing quantum dots or nanoparticles emitting light in a wavelength known to activate protoporphyrin IX or deoxygenated hemoglobin are injected into the tumor bearing host 5-40 minutes before the infusion of the SS cells, SS progenitors and erythroleukemia cells transduced with oncolytic virus. Optionally the SS cells, SS progenitors or erythroleukemia cells are exposed to photosensitizers for a period of 1-60 minutes before light radiation is commenced. The quantum dots and nanoparticles localize in the tumor. The SS cells, progenitors or erythroleukemia cells co-localize in the tumor and undergo photooxidation and lysis by the light emitting particles situated in the tumor leading with release of oncolytic virus into the tumor milieu. Sublethal light exposure or x-irradiation applied to the cells ex vivo before infusion ensures hemolysis and facilitates viral shedding from the cell once the SS cell, SS erythroblast or erythroleukemia cell is deposited in the tumor neovasculature.

SS Cells, Ss Erythroblasts and Erythroleukemia Cells with a Porphyric Phenotype and Porphyria Cells with an SS Phenotype

In an additional embodiment, the gene encoding aminolevulinic acid deamidase is silenced via a siRNA in an SS hematopoietic progenitor cell; alternatively, the SS globin gene is inserted into erythroid progenitor cells from patients with porphyria cutanea tarda, erythrogenic porphyria or acute intermittent porphyria; cells from variants of these diseases or other diseases which over-produce photosensitizing porphyrins are also useful in this invention. These cells produce an abundance of photosensitizing porphyrins including protoporphyrin IX that are activated by visible light resulting in photooxidative hemolysis.

In the present invention, these cells porphyric progenitor cells are transduced by oncolytic virus or nucleic acids encoding tumor/angio-specific immunoglobulins (e.g., anti-VEGF agents). Likewise, a drug resistant population may be produced by exposure to antitumor agents in vivo as described above. These porphyric progenitor cells (10⁴-10¹¹) are optionally exposed to visible light at wave lengths of 200-900 nM for 10 minutes and then administered parenterally to tumor-bearing hosts where they deposit in the tumor neovasculature. The cell undergoes photohemolysis within 30 minute after parenteral administration with consequent shedding of the oncolytic virus, antitumor drug or tumor-specific/neovascular (VEGF)-specific monoclonal antibody into the tumor milieu. The kinetics of cell injury as a function of light exposure (wave length and duration) are determined beforehand so that the ex vivo light-induced photo-oxidation reaches a t½ of 20-60 minutes after administration when the cells are deposited in the tumor neovasculature.

Liposome, Nanoparticles, Quantum Dots, Plasmonic Nanostructures Coupled to Quantum Dots Expressing BCAM/Lu, Alone or Together with ICAM-4, α4β1, or Other Adhesion Molecules to Promote Binding to Tumor Neovasculature

Colloidal semiconductor quantum dots are single crystals a few nanometers in diameter exhibiting composition and size-dependent absorption and emission. Their size and shape can be precisely controlled by the duration, temperature, and ligand molecules used in the synthesis. Absorption of a photon with energy above the semiconductor band gap energy results in the creation of an electron-hole pair (or exciton), an increased probability at higher energies (i.e., shorter wavelengths) resulting in a broadband absorption spectrum. The radiative recombination of an exciton (characterized by a long lifetime, >10 ns) leads to the emission of a photon in a narrow, symmetric energy band. Qdots tend to be brighter than dyes because their extinction coefficients are an order of magnitude larger than most dyes. The long fluorescence lifetime of Qdots enables the use of time-gated detection and the ability to express a specific excitation wavelength allows for the separation of their signal from that of shorter lived species such as background autofluorescence encountered in cells. Their increased brightness and photostability coupled with their ability to emit light in a narrow spectrum make them well suited to selectively activate the denatured hemoglobin, heme and protoporphyrin IX in SS cells, their progenitors and erythroleukemia cells.

Qdot ligands containing either an amine or a carboxyl group, for instance, offer the possibility of cross-linking molecules containing a thiol group or an N-hydroxysuccinimyl ester moiety by means of standard bioconjugation reactions. Another approach uses electrostatic interactions between Qdots and charged adapter molecules, or between Qdots and proteins modified to incorporate charged domains. Different types of functionalization have also been explored as a way to target Qdots to cell surface proteins. Some examples include streptavidin, antibodies, receptor ligands such as epidermal growth factor (EGF) or serotonin, recognition peptides, and affinity pairs such as biotin-avidin permitting the labeling of most types of target in vivo.

Biologically synthesized Qdots have CdS cores coated by natural peptides. Peptides have the advantage of (i) protecting the core/shell structure and maintain the original Qdot photophysics, (ii) solubilizing Qdots, (iii) providing a biological interface, and (iv) allowing the incorporation of multiple functions. The resulting particles have excellent colloidal properties, photophysics, and biocompatibility, and this peptide toolkit can easily be tailored to provide additional functionalities.

The present invention contemplates that mature SS cells localizing in the tumor vasculature express at least three potent receptor systems BCAM/Lu, ICAM-4 and α4β1 whose cognate ligands are known to be expressed on the tumor neovasculature. These three receptors are coupled to derivatized liposomes, nanoparticles, Qdots or other non-viable biocompatible particles suitable for in vivo use preferably using bifunctional and methodology described below and conjugating agents described in Table 3. Each particle may contain individual receptors or a mixture of two or more receptors to mimic the distribution on a viable SS cell or progenitor. For in vivo administration, particles containing at least one of the three receptors or a mixture of particles each containing one, two or all three receptors are administered parenterally. Upon infusion into tumor bearing hosts, these particles localize in the tumor vasculature.

Quantum dots with tumor targeting molecules such as tumor specific antibodies, receptors or ligands conjugated to them are prepared using derivatization procedures and bifunctional crosslinkers as described below. These molecules are then infused into the tumor bearing host where they localize in the tumor. Five to 60 minutes later, SS cells, SS erythroblasts or erythroleukemia cells transfected with oncolytic virus or chemotherapy are administered. The cells localize to the tumor where they are are activated by the previously infused and tumor-bound Qdots. The latter selectively emit light in a spectral range that activates denatured hemoglobin and protoporphyrin X present in the SS cells, SS erythroblasts or erythroleukemia cells. The latter cells lyse releasing their oncolytic virus or chemotherapy into the tumor milieu.

Directing light waves at the interface between a metal and dielectric can induce a resonant surface of the metal. This results in the generation of surface plasmons-density waves of electrons that propagate along the interface. Using a plasmon slot waveguide and adjusting the thickness of the dielectric core, the wavelength of the plasmons can transmit a signal as far as tens of microns. They can also generate signals in the soft x-ray range of wave lengths (between 10 and 100 nanometers) by exciting materials with visible light. Coating the surface of silicon Qdots with silver or gold plasmonic nanostructures boosts their light emission by about 10 fold. Plasmonic nanostructures with stronger light emission than Qdots are coupled to tumor targeting moieties (as described above) and preferred over quantum dots for tumor localization after parenteral administration. These particles are derivatized as described below, coupled to BCAM/Lu, (optionally to α4β1 and ICAM-4) and administered parenterally to the host wherein they localize to tumors in vivo. They are readily detected by external sources as described below for diagnostic purposes or they may be activated in a therapeutic context activated by an external light source. Their brighter emission allows the BCAM/Lu plasmonic Qdots to be readily detected in small metastatic foci of tumor by a total body scanner. An external light source capable of penetrating tissues activates them generating tumor temperatures above 42° C. resulting in tumor cell killing selectively at metastatic sites.

SS cells, SS erythroblasts and erythroleukemia cells (optionally pre-photosensitized ex vivo before administration as described above and transfected with oncolytic viruses or chemotherapy) are administered 10-60 minutes after the above tumor targeted plasmonic particles have deposited in tumor. The SS cells, SS erythroblasts and erythroleukemia cells deposit in the tumor neovasculature are hemolyzed by the bright light of the tumor-bound plasmonic particles emitting selectively in a range known to activate denatured hemoglobin or protophoryrin X overexpressed in the SS cells, SS erythroblasts and erythroleukemia cells.

### Preparation of Quantum Dots

Quantum dots obtained from Quantum Dot Corp. QD605 and QD655 have typical CdSe/ZnS core-shell structures, and QD705 and QD800 are made of CdTe cores with ZnS coatings. The organic coating chemistry has been previously described in the literature, and the final coated quantum dots are endowed with carboxylate groups. The quantum yields of each quantum dot determined in 50 mM borate buffer (pH 9) are 65% (QD605), 83% (QD655), 80% (QD705) and 43% (QD800). The hydrodynamic diameters of all quantum dots and conjugates are measured by Malvern Instruments Ltd. with a Zetasizer Nano ZS. The coupling reagent 1-ethyl-3-(3-dimethylamino-propyl)carbodiimide hydrochloride (EDC) is from Fluka.

For solubilization, their hydrophobic surface ligands are replaced by amphiphilic ones. Different Qdot solubilization strategies include (i) ligand exchange with simple thiol-containing molecules or oligomeric phosphines, dendrons, and peptides; (ii) encapsulation by a layer of amphiphilic diblock or triblock copolymers or in silica shells phospholipid micelles, polymer beads, polymer shells, or amphiphilic polysaccharides; and (iii) combinations of layers of different molecules conferring the required colloidal stability to Qdots. A water-based synthesis method yielding particles that emit from the visible to the NIR spectrum are intrinsically water-soluble.

### Preparation of QD Conjugates.

Method 1. Organic-soluble, CdSe/ZnS core-shell nanocrystals (Hines, M. A. & Guyot-Sionnest, P. J. Phys. Chem. 100: 468-471 (1996); Dabbousi. B. O. et al. J. Phys. Chem. 101: 9463-9475 (1997) are isolated from hexanes and ligand solution with an equal volume of methanol, rinsed with methanol, and redispersed in CHCl₃. These materials are mixed with neutralized amphiphilic polymer (40% octylamine-modified polyacrylic acid, 2,000 units/QDot) in CHCl₃, and the solvent evaporated. The dry film is redispersed in water and purified from excess polymer by gel filtration. The surface coating is cross-linked further by EDC (1-ethyl-3-(3-dimethylamino propylcarbodiimide)-mediated coupling to lysine (or polyethylene glycol-lysine), and these materials are then coupled to streptavidin or antibodies by an EDC-mediated coupling reaction in 10 mM borate buffer, pH 8.0. QDot bioconjugates are diluted for use in 10 mM borate buffer, pH 8.2. QD 535, QD 560, QD 608, and QD 630 are used.
Method 2. To a mixture of 8.2 pmol of Qdots and 164 pmol of peptide (20 equivalents) in 200 ul borate buffer (pH 7.4), 32.8 nmol of EDC (4,000 equivalents) are added. Borate buffer is chosen to minimize quantum dot aggregation during the coupling. The mixture is incubated for 1 h, and the uncoupled free peptide or protein conjugate partner and excess EDC are removed by three washes using a 100 K NanoSep filter (Pall Corporation) by centrifugation at 2655 g for 3 min at 4° C. The final complex is kept in borate buffer at 4° C.

### Biochemical Cross-Linkers

Receptor molecules specific for the tumor neovasculature are linked directly to a nanoparticles, liposomes or Qdots via certain preferred biochemical linker or spacer groups. For chemical conjugates, cross-linking reagents are preferred and are used to form molecular bridges that bond together functional groups of two different molecules. Heterobifunctional crosslinkers can be used to link two different proteins in a step-wise manner while preventing unwanted homopolymer formation. Such cross-linkers are listed in Table 3, below.

Hetero-bifunctional cross-linkers contain two reactive groups one (e.g., N-hydroxy succinimide) generally reacting with primary amine group and the other (e.g., pyridyl disulfide, maleimides, halogens, etc.) reacting with a thiol group. Compositions to be crosslinked therefore generally have, or are derivatized to have, an available functional binding group. This requirement is not considered to be limiting in that a wide variety of groups can be used in this manner. For example, primary or secondary amine groups, hydrazide or hydrazine groups, carboxyl, hydroxyl, phosphate, or alkylating groups may be used for binding or cross-linking.

The spacer arm between the two reactive groups of a cross-linker may be of various length and chemical composition. A longer, aliphatic spacer arm allows a more flexible linkage while certain chemical groups (e.g., benzene group) lend extra stability or rigidity to the reactive groups or increased resistance of the chemical link to the action of various agents (e.g., disulfide bond resistant to reducing agents). Peptide spacers, such as Leu-Ala-Leu-Ala, are also contemplated.

It is preferred that a cross-linker have reasonable stability in blood. Numerous known disulfide bond-containing linkers can be used to conjugate two polypeptides. Linkers that contain a disulfide bond that is sterically hindered may give greater stability in vivo, preventing release of the agent prior to binding at the desired site of action.

A most preferred cross-linking reagents for use in with antibody chains is SMPT, a bifunctional cross-linker containing a disulfide bond that is “sterically hindered” by an adjacent benzene ring and methyl groups. Such steric hindrance of the disulfide bond may protect the bond from attack by thiolate anions (e.g., glutathione) which can be present in tissues and blood, and thereby help in preventing decoupling of the conjugate prior to the delivery to the target, tumor site. SMPT cross-links functional groups such as —SH or primary amines (e.g., the ∈-amino group of Lys).

TABLE 3
Hetero-Bifunctional Cross-linkers

| | | Spacer arm length |
|---|---|---|
| Linker | Advantages and Applications | after cross linking |
| Succinimidyloxycarbonyl-α-(2- | Greater stability | 11.2 A |
| pyridyldithio)toluene (SMPT) 1 | | |
| N-succinimidyl 3-(2- | Thiolation | 6.8 A |
| pyridyldithio)propionate (SPDP) 2 | | |
| Sulfosuccinimidyl-6-[α-methyl-α-(2- | Extended spacer arm; Water-soluble | 15.6 A |
| pyridyldithio)toluamido]hexanoate | | |
| (Sulfo-LC-SPDP)1 | | |
| Succinimidyl-4-(N- | Stable maleimide reactive group; | 11.6 A |
| maleimidomethyl)cyclohexane-1- | conjugation of enzyme or other | |
| carboxylate (SMCC)1 | polypeptide to antibody | |
| Succimimidyl-4-(N- | Stable maleimide reactive group; water- | 11.6 A |
| maleimidomethyl)cyclohexane- | soluble | |
| carboxylate (Sulfo-SMCC) 1 | | |
| m-Maleimidobenzoyl-N- | Enzyme-antibody conjugation; hapten- | 9.9 A |
| hydroxysuccinimide (MBS) 1 | carrier protein conjugation | |
| m-Maleidmidobenzoyl-N- | Water-soluble | 9.9 A |
| hydroxysulfosuccinimide (Sulfo- | | |
| MBS) 1 | | |
| N-Succinimidyl(4- | Enzyme-antibody conjugation | 10.6 A |
| iodacetyl)aminobenzoate (SIAB) 1 | | |
| Sulfosuccinimidyl(4- | Water-soluble | 10.6 A |
| iodoacetyl)aminobenzoate (Sulfo- | | |
| SIAB) 1 | | |
| Succinimidyl-4-(p- | Enzyme-antibody conjugation; extended | 14.5 A |
| maleimidophenyl)butyrate (SMPB) 1 | spacer arm | |
| Sulfosuccinimidyl-4-(p- | Extended spacer arm | 14.5 A |
| maleimidophenyl)butyate (Sulfo- | Water-soluble | |
| SMPB) 1 | | |
| 1-ethyl-3-(3- | Hapten-Carrier conjugation | 0 |
| dimethylaminopropyl)carbodiimide | | |
| hydrochloride N- | | |
| Hydroxysulfosuccinimide | | |
| (EDC/Sulfo-NHS) 3 | | |
| p-Azidobenzoyl hydrazide (ABH) 4 | Reacts with sugar groups | 11.9 A |

1 Reactive toward primary amines, sulfhydryls
2 Reactive toward primary amines
3 Reactive toward primary amines, carboxyl groups
4 Reactive toward carbohydrates, nonselective

Hetero-bifunctional photoreactive phenylazides containing a cleavable disulfide bond, for example, sulfosuccinimidyl-2-(p-azido salicylamido)-ethyl-1,3-dithiopropionate. The N-hydroxy-succinimidyl group reacts with primary amino groups and the phenylazide (upon photolysis) reacts non-selectively with any amino acid residue. Other useful cross-linkers, not considered to contain or generate a protected disulfide, include SATA, SPDP and 2-iminothiolane. The use of such cross-linkers is well known in the art.

Once conjugated to the QDot, liposome or nanoparticle, the conjugate is separated from unconjugated receptors or partner polypeptides and from other contaminants. A large number of purification techniques are available for use in providing conjugates of a sufficient degree of purity to render them clinically useful. Purification methods based upon size separation, such as gel filtration, gel permeation or high performance liquid chromatography will generally be of most use. Other chromatographic techniques, such as Blue-Sepharose separation, may also be used.

The preferred targeting molecule for incorporation into Qdots, nanoparticles and liposomes is BCAM/Lu which may optionally be combined with other adhesion molecules, ICAM-4 and/or α4β1 or any other molecule known to bind to tumors or tumor neovasculature. Their amino acid sequences are given below.

| BCAM/Lu Parsons SF et al., Proc. Natl. Acad. Sci. | |
|---|---|
| 92: 5496-5500 (1995) | |
| (SEQ ID NO: 66) | |
| 1 | meppdapaqa rgaprlllla vllaahpdaq aevrlsvppl vevmrgksvi |
| | ldctptgthd |
| 61 | hymlewfltd rsgarprlas aemqgselqv tmhdtrgrsp pyqldsqgrl |
| | vlaeaqvgde |
| 121 | rdyvcvvrag aagtaeatar lnvfakpeat evspnkgtls vmedsaqeia |
| | tcnsrngnpa |
| 181 | pkitwyrngq rlevpvemnp egymtsrtvr easgllslts tlylrlrkdd |
| | rdasfhcaah |
| 241 | yslpegrhgr ldsptfhltl hyptehvqfw vgspstpagw vregdtvqll |
| | crgdgspspe |
| 301 | ytlfrlqdeq eevlnvnleg nltlegvtrg qsgtygcrve dydaaddvql |
| | sktlelrvay |
| 361 | ldplelsegk vlslplnssa vvncsvhglp tpalrwtkds tplgdgpmls |
| | lssitfdsng |
| 421 | tyvceaslpt vpvlsrtqnf tllvqgspel ktaeiepkad gswregdevt |
| | licsarghpd |
| 481 | pklswsqlgg spaepipgrq gwvsssltlk vtsalsrdgi sceasnphgn |
| | krhvfhfgtv |
| 541 | spqtsqagva vmavavsvgl lllvvavfyc vrrkggpccr qrrekgappp |
| | gepglshsgs |
| 601 | eqpeqtgllm ggasggargg sggfgdec |
| ICAM-4 Bailly P et al., Proc. Natl. Acad. Sci. | |
| 91: 5306-5310 (1994) | |
| (SEQ ID NO: 67) | |
| 1 | mgslfplsll fflaaaypgv gsalgrrtkr aqspkgspla psgtsvpfwv |
| | rmspefvavq |
| 61 | pgksvqlncs nscpqpqnss lrtplrqgkt lrgpgwvsyq lldvrawssl |
| | ahclvtcagk |
| 121 | trwatsrita ykpphsvile ppvlkgrkyt lrchvtqvfp vgylvvtlrh |
| | gsrviysesl |
| 181 | erftgldlan vtltyefaag prdfwqpvic harlnldglv vrnssapitl |
| | mlawspapta |
| 241 | lasgsiaalv gilltvgaay lckclamksq a |
| α4β1 Takada, Y. et al. EMBO J. 8: | |
| 1361-1368 (1989) | |
| (SEQ ID NO: 68) | |
| 1 | mawearrepg prraavretv mlllclgvpt grpynvdtes allyqgphnt |
| | lfgysvvlhs |
| 61 | hganrwllvg aptanwlana svinpgaiyr crigknpgqt ceqlqlgspn |
| | gepcgktcle |
| 121 | erdnqwlgvt lsrqpgengs ivtcghrwkn ifyiknenkl ptggcygvpp |
| | dlrtelskri |
| 181 | apcyqdyvkk fgenfascqa gissfytkdl ivmgapgssy wtgslfvyni |
| | ttnkykafld |
| 241 | kqnqvkfgsy lgysvgaghf rsqhttevvg gapqheqigk ayifsideke |
| | lnilhemkgk |
| 301 | klgsyfgasv cavdlnadgf sdllvgapmq stireegrvf vyinsgsgav |
| | mnametnlvg |
| 361 | sdkyaarfge sivnlgdidn dgfedvaiga pqeddlqgai yiyngradgi |
| | sstfsqrieg |
| 421 | lqiskslsmf gqsisgqida dnngyvdvav gafrsdsavl lrtrpvvivd |
| | aslshpesvn |
| 481 | rtkfdcveng wpsvcidltl cfsykgkevp gyivlfynms ldvnrkaesp |
| | prfyfssngt |
| 541 | sdvitgsiqv ssreancrth qafmrkdvrd iltpiqieaa yhlgphvisk |
| | rsteefpplq |
| 601 | pilqqkkekd imkktinfar fcahencsad lqvsakigfl kphenktyla |
| | vgsmktlmln |
| 661 | vslfnagdda yettlhvklp vglyfikile leekqincev tdnsgvvqld |
| | csigyiyvdh |
| 721 | lsridisfll dvsslsraee dlsitvhatc eneeemdnlk hsrvtvaipl |
| | kyevkltvhg |
| 781 | fvnptsfvyg sndenepetc mvekmnltfh vintgnsmap nvsveimvpn |
| | sfspqtdklf |
| 841 | nildvqtttg echfenyqrv caleqqksam qtlkgivrfl sktdkrllyc |
| | ikadphclnf |
| 901 | lcnfgkmesg keasvhiqle grpsilemde tsalkfeira tgfpepnprv |
| | ielnkdenva |
| 961 | hvlleglhhq rpkryftivi issslllgli vlllisyvmw kagffkrqyk |
| | silqeenrrd |
| 1021 | swsyinsksn dd |
| 721 | lsridisfll dvsslsraee dlsitvhatc eneeemdnlk hsrvtvaipl |
| | kyevkltvhg |
| 781 | fvnptsfvyg sndenepetc mvekmnltfh vintgnsmap nvsveimvpn |
| | sfspqtdklf |
| 841 | nildvqtttg echfenyqrv caleqqksam qtlkgivrfl sktdkrllyc |
| | ikadphclnf |
| 901 | lcnfgkmesg keasvhiqle grpsilemde tsalkfeira tgfpepnprv |
| | ielnkdenva |
| 961 | hvlleglhhq rpkryftivi issslllgii vlllisyvmw kagffkrqyk |
| | silqeenrrd |
| 1021 | swsyinsksn dd |

### In Vivo Fluorescence Imaging.

Quantum dot conjugated to BCAM/Lu are injected either subcutaneously, intramuscularly or intravenously into tail vein of nude mice. Images are acquired with and without filters. Mice are subsequently anesthetized with isoflorane, and transferred into the light-tight chamber of an IVIS 200 imager. Wavelength-resolved spectral imaging is carried out using a spectral imaging system (Maestro In-Vivo Imaging System from Cambridge Research & Instrumentation). The excitation filter is 503-555 nm. The tunable filter is automatically stepped in 10-nm increments from 580 to 900 nm with an exposure time of 49 ms for images captured at each wavelength. Animals are placed supine under isofluorane anesthesia in a light-tight chamber. Collected images are analyzed by the Maestro software, using spectral unmixing algorithms to separate autofluorescence from Qdot signals. The in vitro multiplexed bioluminescence imaging of quantum dot conjugates is performed similarly with the Maestro system, but with the excitation light blocked and 5-s exposure time for each individual acquisition.

### Sickle Cell Ghosts, SS Erythrocytes, SS Progenitors and Erythroleukemia Cells as Carriers of Tumoricidal Agents

The extremely plastic structure of the erythrocyte and the ability to remove its cytoplasmic contents and reseal the plasma membranes enable the entrapment of different macromolecules within the so-called hemoglobin free “ghost.” Combining these ghosts and a fusogen such as polyethylene glycol has permitted the introduction of a variety of macromolecules into mammalian cells (Wiberg, F C et al., Nucleic Acid Res. 11: 7287-7289 (1983); Wiberg, F C et al., Mol. Cell. Biol. 6: 653-658 (1986); Wiberg, F C et al., Exp. Cell. Res. 173: 218-227 (1987)). The mature sickled erythrocyte, SS progenitor cells and erythroleukemia cells can be modified in this way and still retain its rigid membrane structure. Thus it can be used to entrap tumoricidal agents, oncolytic viruses and plasmids encoding oncolytic viruses, toxins, toxin-antibody conjugates, therapeutic monoclonal antibodies and carry them into the tumor vasculature. Tumor killing agents introduced into the sickled erythrocyte are released locally following deposition in the tumor microcirculation. Some of the most promising agents include spores of Clostridia perfringens novyi a non-pathogenic anaerobic bacteria selectively activated in anaerobic tissue has shown tumoricidal activity in murine models (Dang et al., Proc. Natl. Acad. Sci. 98: 15155-15160 (2001), non-pathogenic Listeria monocytogenes which specifically activates tumor killing (T_{H}-1) cytokines and also produces a hemolysin (listerolysin) or dead but metabolically active listeria or other bacterial species that enables it to lyse the SS erythrocytes from within the cell (Brockstedt et al., Nat. Med. 11:853-60 (2005)). Modified bacteria are incorporated into the erythrocytes by fusion of the bacteria with erythrocyte membrane followed by internalization. Anaerobic spores such Clostridia novyi are encapsulated by sickled erythrocytes, SS progenitors, or erythroleukemia cells stably transfected with BCAM/Lu by the methods of Schrier S. Meth. Enzymol. 149: 261-271 (1987) and Tsong T Y Meth. Enzymol. 149:248-259 (1987); Deloach J R Meth. Enzymol. 149: 235-242 (1987)); however, any other encapsulation procedure described below or in Methods in Enzymology, vol 149, Academic Press, New York, N.Y. (1997) herein incorporated by reference in their entirety is useful in the present invention. Anti-tumor drugs especially those active under anaerobic conditions can be also be encapsulated in this fashion. Phage displays, exosomes, sickle cell vesicles, yeast sec vesicles expressing tumoricidal toxins or superantigens can be prepared and incorporated into mature sickled erythrocytes by fusigenic methods previously described. These cells loaded with spores are preferentially exposed to photosensitizers and a light source as described above to induce a VA cell lysis of 10-60 minutes. They are administered to tumor bearing hosts, deposit in the tumor neovasculature, undergo photolysis and release their contents in the tumor milieu.

Various types of chemotherapy can be loaded into mature sickled erythrocytes or erythrocyte ghosts preferably before administration some which have particular effectiveness in the hypoxemic micro-environment of the tumor. These include quinone-containing alkylating agents, of which mitomycin C is the prototype and nitroaromatic compounds, of which misonidazole and RB 6145 are examples. Tirapazamine is the prototype hypoxia-activated prodrug and is particularly useful. Its toxic metabolite is a highly reactive radical present at higher concentrations under hypoxia that selectively kills radio-resistant hypoxic cells in tumors. This makes the tumors much more sensitive to treatment with conventional chemotherapy and radiotherapy. An additional chemotherapeutic useful in this invention is dolostatin an antivascular agent that leads to vascular shutdown in tumors and traps molecules such as sickled erythrocytes with their tumoricidal loads in the tumor microvasculature. Indeed, various antineoplastic drugs such as actinomycin D, bleomycin and cytosine arabinoside are entrapped in erythrocyte ghosts by well established methods (Deloach & Barton C. Am. J. Vet. Res. 42: 1971 (1981); Deloach & Barton C. Am. J. Vet. Res. 43:2210 (1983); Lynch W S et al., Am. J. Hematol. 9: 249 (1980)). Normally, the drug is added externally and incorporated inside the erythrocyte by a passive mechanism. However, if the molecular weight of the substance to be encapsulated is greater than the cutoff of the dialysis tube, the drug is added to the erythrocytes before dialysis. If available in limited amounts the drug is incubated directly after the dialysis step with the dialyzed erythrocytes. Here, the cells are preferentially photosensitized and exposed to light ex vivo as described above and then administered to tumor bearing hosts where they localize in tumor neovasculature.

SS ghosts from mature SA, SS erythrocytes from patients with sickle cell trait or sickle cell anemia respectively are useful for encapsulation of anaerobic bacteria such as Clostridia novyi, Listeria or S. aureus because under physiologic conditions they show normal morphology whereas under the more extreme conditions of hypoxia such as the acidotic and/or hypoxemic tumor microvasculature they sickle and become adherent to the microvasculature. Once adherent to the endothelium of the tumor microcirculation, they obstruct microvasculature in a manner similar to the homozygous SS erythrocytes.

The present invention contemplates sickle cell ghosts, SS erythrocytes, their precursors, variants and erythroleukemia cells as carriers of chemotherapy, prodrugs, anti-tumor angiogenic therapy, tumoricidal proteins, toxins, e.g., superantigens, diphtheria, ricin, pseudomonas exotoxin A and toxin-tumor specific antibody conjugates, tumor specific antibodies, enzymes and metals such as iron and gold selectively into tumors. They can be carriers of Qdots, liposomes and nanoparticles or any other type of biocompatible particle with tumor localizing properties.

### SS Cell Encapsulation Methodology

### Collection and Washing of Erythrocytes

The methods to encapsulate drugs, enzymes or peptides are based on the property of the RBC to increase in volume when placed under conditions of reduced osmotic pressure, such as in the presence of a hypotonic solution. The hypotonic encapsulation method of Deloach Jr et al., Am. J. Vet Res. 42: 667-671 (1981) considered to be representative of the field and preserves the biochemical and physiological characteristics of the erythrocytes and the highest percentage of encapsulation.

Carrier erythrocytes may be prepared from human blood and blood of different animal species such as rat, mouse, rabbit, dog, etc. Blood is taken from patients with sickle cell anemia with homozygous SS hemoglobin and the erythrocytes collected using an appropriate anticoagulant such as EDTA or a mixture of citrate, phosphate and dextrose (CPD) because it best preserves the properties of red blood cells although some use heparin (1000 IE/10 ml blood). Erythrocytes are separated from serum and buffy coat by centrifugation at room temperature and washed 4 times in via centrifugation (530 g, 15 min, 4° C.) with isotonic solutions usually Hanks-PBS buffer to remove other blood components. It is also possible to achieve a good washing with a plasma separator.

### Dialysis of Erythrocytes Against Hypotonic Buffer

This step allows substances to enter the red cells by an increase in porosity due to the hypotonic environment. Washed packed erythrocytes (hematocrit 50-90%; 5 to 10 ml) are placed inside a dialysis bag. The substance to be encapsulated is added either to the actual suspension of erythrocytes, adjusting the final hematocrit of the suspension, or dissolved in the external dialysis buffer. Dialysis is carried out with an appropriate hypotonic buffer at 48° C., pH of 7.4 and continued for various periods. Single dialysis membranes with a molecular cutoff of 3.4-14 kDa, are useful although two types of membrane with different molecular weight cutoffs are recognized in the art. Tonicity is restored by addition of sufficient quantity of 154 mM NaCl to bring the osmolality up to 300 mOsm. By raising the salt concentration to its original level, the RBC pores close, the RBCs reassume their normal biconcave shape and the substance remains encapsulated inside the cells at a suitable concentration. Nonentrapped substances are washed out and the loaded RBCs are ready to be used as carriers for the delivery of the encapsulated drugs (Rossi et al., Expert Opin. Drug Deliv. 2: 311-322 (2005)).

The art recognizes that composition and osmolality of the buffers may vary depending on the animal species employed and the substance to be encapsulated exemplified in Table 2 of Rossi et al., Expert Opin. Drug Deliv. 2: 311-322 (2005). For human erythrocytes, the osmolalities of the hypotonic buffers vary from 26 to 220 mOsm/kg based on the substance to be encapsulated (see Table 3 of Rossi et al., Expert Opin. Drug Deliv. 2: 311-322 (2005)). The duration of the dialysis for human erythrocytes ranges between 20 and 180 min. Additionally, the volume ratio (v/v) between the erythrocyte suspension and the dialysis buffer of 1:50 is effective. Automated systems with dialysate flow rates ranging from 15-19 ml/min to 20-60 ml/min. are useful.

Several buffers are used to wash the erythrocytes before and after dialysis. These include but are not limited to (i) 154 mM NaCl; (ii) 15 mM Na₂HPO (pH 7.0), 10 nM glucose, 144 mM NaCl (phosphate butter); (iii) 1.5 mM NaCl pH 7.0), 10 mM glucose, 144 mM NaCl (phosphate-MgCl₂ buffer); (iv) 5 mM Na₂HPO₄, pH 7.0, 0-5 mM CaCl₂, 10 mM glucose, 154 mM NaCl (phosphate-CaCl₂ buffer; (v) 10 mM Tris HCl (pH 7.0), 10 nM glucose, 144 mM NaCl: and (vi) 154 mM NaCl, 5 mM MgCl₂ (NaCl—MgCl₂ buffer).

### Resealing Step

The resealing step produces entrapment and encapsulation of a drug, enzyme, antibody, polypeptide by closing the RBC pores. The dialysis bag containing the erythrocyte suspension is transferred to an isotonic or hypertonic buffer isotonic buffer such as Hanks-PBS for 10 min at 37° C., or a highly hypertonic buffer is added at a proportion of 0.1:1 (v/v) directly to the erythrocyte suspension. The buffer compositions employed in the resealing step are given in Table 2 of Gutierrez Millan C et al., Blood Cells, Molecules, Diseases 33: 132-140 (2004). After resealing, the erythrocytes are washed several times with an isotonic buffer at 48° C. and then resuspended in plasma for later reinjection. Washing with hypotonic buffers leads to the removal of the most fragile carrier cells.

Quantitative range of reagents used in the performance of encapsulation of various molecules within intact human RBCs is a summarized in Table 4 (FIG. 1, from Gutierrez Millan C et al., Blood Cells Molecules, Diseases 33: 132-140 (2004). Technical modifications may vary with the physical properties of the species of molecule to be encapsulated but are well within the skill of the ordinary scientist

TABLE 4
Range of Conditions Used for Encapsulation of Human Erythrocytes

| | Buffer | Temperature | Time range |
|---|---|---|---|
| Washing | isotonic | 4° C. | 5-15 min |

| | | | Dialysis | | Volume | Hematocrit |
|---|---|---|---|---|---|---|
| | Buffer | Temperature | Time range | pH | ratio range | range |
| Hypotonic | 26-220 mOsm/kg | 4° C. | 20-180 min | 7.4 | 1/10- 1/300 | 50-90% |
| Dialysis | | | | | | |

| | | | Annealing | |
|---|---|---|---|---|
| | Buffer | Temperature | Time range | pH |
| Annealing | isotonic | 25-39° C. | 10-30 min | 7.4 |

| | Volume | | Resealing | |
|---|---|---|---|---|
| | ratio range | Temperature | time range | pH |
| Resealing | 1/10- 1/30 | 4-39° C. | 5-60 min | 7.4 |

| | Buffer | Temperature | Time range |
|---|---|---|---|
| Washing | isotonic | 4° C. | 5-15 min |

Encapsulation Protocol is Given in Example 7.

Before administration in vivo, the red cell ghosts encapsulating the desired therapeutic substance prepared by the above methods are optionally exposed to a light source in a wave length of 50-900 nanometers for 5-60 minutes designed to induce a photohemolysis t½ of 20-60 minutes. Following parenteral administration red cell ghosts localize in tumor neovasculature where they undergo photohemolysis shedding their contents into the tumor milieu. The ghosts may also be coencapsulated with small amounts of ferrous particles and hemoglobin or exposed exogenously to photosensitizers and light ex vivo as described above to ensure timely photohemolysis after the cells are administered in vivo and localized in the tumor bed.

Vesicles from sickled erythrocytes are shed from the parent cells. They contain membrane phospholipids which are similar to the parent cells but are depleted of spectrin. They also demonstrate that a shortened Russell's viper venom clotting time by 55% to 70% of control values and become more rigid under acid pH conditions. Rigid sickle cell vesicles induce hypercoagulability. Vesicles shed from immature or mature sickled erythrocytes are capable of localizing to tumor microvascular sites where they bind and induce an anti-tumor effect.

Vesicles are prepared and isolated as follows: Blood is obtained from patients with homozygous sickle cell anemia. The PCV range is 20-30%, reticulocyte range is 8-27%, fetal hemoglobin range is 25-13% and endogenous level of ISCs is 2-8%. Blood is collected in heparin and the red cells are separated by centrifugation and washed three times with 0.9% saline. Cells are incubated at 37° C. and 10% PCV in Krebs-Ringer solutions in which the normal bicarbonate buffer is replaced by 20 mM Hepes-NaOH buffer and which contains either 1 mM CaCl₂ or 1 mM EGTA. All solutions contain penicillin (200 U/ml) and streptomycin sulphate (100 ug/ml). Control samples of normal erythrocytes are incubated in parallel with the sickle cells. Incubations of 10 ml aliquots are conducted in either 100% N₂ or in room air for various periods in a shaking water bath (100 oscillations per mm). N₂ overlaying is obtained by allowing specimens to equilibrate for 45 mm in a sealed glove box (Gallenkamp) which was flushed with 100% N₂. Residual oxygen tension in the sealed box is less than 1 mmHg. The percentage of irreversibly sickled cells is determined by counting. 1000 cells after oxygenation in room air for 30 mm and fixation in buffered saline (130 mM NaCl, 20 mM sodium phosphate, pH 74) containing 2% glutaraldehyde. Cells whose length is greater than twice the width and which possessed one or more pointed extremities under oxygenated conditions are considered to be irreversibly sickled. After various periods of incubation, cells are sedimented at 500 g for 5 mm and microvesicles) are isolated from the supernatant solution by centrifugation at 15,000 g for 15 mm. The microvesicles form a firm bright red pellet sometimes overlain by a pink, flocculent pellet of ghosts (in those cases where lysis was evident) which is removed by aspiration.

Quantitation of microvesicles is achieved by resuspension of the red pellet in 1 ml of 05% Triton X followed by measurement of the optical density of the clear solution at 550 nm. Optical density measurements at 550 nm give results that are relatively the same as measurements of phospholipid and cholesterol content in the microvesicles. Cell lysis is determined by measurement of the optical density at 550 nm of the clear supernatant solution remaining after sedimentation of the microvesicles. Larger samples of microvesicles for biochemical and morphological analysis are prepared from both sickle and normal cells following incubation of up to 100 ml of cell suspension at 37° C. for 24 h in the absence or presence of Ca₂⁺⁺. Ghosts are prepared from sickle cells after various periods of incubation. The cells are lysed and the ghosts washed in 10 mM Tris HCl buffer, pH 73, containing O₂ mM EGTA.

The compositions of the claimed invention are useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries, choriocarcinoma and gestational trophoblastic disease; male genital tract including prostate, seminal vesicles, testes and germ cell tumors; endocrine glands including thyroid, adrenal, and pituitary; skin including hemangiomas, melanomas, sarcomas arising from bone or soft tissues and Kaposi's sarcoma; tumors of the brain, nerves, eyes, and meninges including astrocytomas, gliomas, glioblastomas, retinoblastomas, neuromas, neuroblastomas, Schwannomas and meningiomas; solid tumors arising from hematopoietic malignancies such as leukemias and including chloromas, plasmacytomas, plaques and tumors of mycosis fungoides and cutaneous T-cell lymphoma/leukemia; lymphomas including both Hodgkin's and non-Hodgkin's lymphomas.

The compositions are also be useful for the prevention of metastases from the tumors described above either when used alone or in combination with radiotherapeutic, photodynamic, and/or chemotherapeutic treatments conventionally administered to patients for treating disorders, including angiogenic disorders. Treatment of a tumor with surgery, photodynamic therapy, radiation and/or chemotherapy is followed by administration of the compositions to extend the dormancy of micrometastases and to stabilize and inhibit the growth of any residual primary tumor or metastases. The compositions can be administered before, during, or after radiotherapy; before, during, or after chemotherapy; and/or before, during, or after photodynamic therapy.

The present invention contemplates that erythrocytes or erythroblasts from patients with any form of sickle hemoglobinopathy are useful. These include erythrocytes or erythroblasts from hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. Indeed, any erythrocyte or erythroblasts with or without sickle hemoglobin expressing receptors capable of binding to tumor neovasculature are useful in the inventions described herein. Particularly useful are those cells which express hemoglobin S in combination with other types of hemoglobin. Both mature and nucleated forms of these cells are useful. In addition, the present invention contemplates that normal or leukemic erythrocytes or their nucleated progenitors transduced with hemoglobin genes from patients with hemoglobinopathies to produce a cell that behaves substantially like an SS or SA erythrocyte or erythrocyte precursor is useful. The present invention also contemplates that normal or sickle erythrocytes or sickle variants, e.g., HbSC cells, and nucleated progenitors which are upregulated by hormones, cytokines, biologically active agents, drugs, chemical or physical treatments to express adhesive properties or to enhance expression of adhesive properties are also useful in this invention.

Nucleated Erythroleukemia Cells, Transduced with BCAM/Lu and/or Other Adhesions Molecules, Sickle Hemoglobin Genes, Oncolytic Viruses and Tumoricidal Transgenes

Additional nucleated erythrocytes that are useful in this invention are human erythroleukemia cells readily obtained from peripheral blood, bone marrow or tissue cultured cell lines. Human erythroleukemia cells are exemplified by the established human cell line K562. Erythroleukemic K562 cells are immature erythroid cells that can be stably transduced with a broad array of nucleic acids encoding integrins and adhesion molecules. They express α5v1 integrins B-CAM-Lu specific for fibronectin and laminin respectively. Adherence to fibronectin through α5v1 works synergetically with α4β1 receptor.

In sickle erythrocytes, their nucleated progenitors (collectively SS cells) and erythroleukemic cells, adrenergic stimuli and PMA upregulate expression of B-CAM/Lu receptors that are specific and highly avid for laminin-α5 receptors (Udani M et al., J Clin Invest. 101:2550-8 (1998)). Normal erythrocyte BCAM-1/Lu is unaffected by this treatment. Likewise adrenergic stimulation of SS cells but not normal erythrocytes induces increased binding to endothelial cell αvβ3 mediated by ICAM-4(LW, CD242). Stimulation of erythroleukemia cells and SS cells with epinephrine and forskolin increased Lu phosphorylation by PKA selectively and enhanced adhesion to laminin under flow conditions while normal red cells were unaffected (Hines P C et al., Blood. 101:3281-7 (2003); Gauthier E et al., J Biol. Chem. 280:30055-62 (2005); Zennadi R et al., Blood 104:3774-81 (2004)). Therefore, SS cells, nucleated precursors and sickle hemoglobin variants stimulated with epinephrine (or similar adrenergic agents) to upregulate BCAM-/Lu and ICAM-4 receptor binding to tumor endothelial laminin α5 and αvβ3 respectively, are particularly useful to increase deposition of these cells in the tumor microvasculature. Likewise, erythroleukemia cells stimulated with adrenergic agents and/or transduced by nucleic acids encoding BCAM/lu are useful in this invention.

K562-expressed α₅β₁ integrin is predominantly in an inactive state. However, addition of stimulatory anti-β₁-integrin antibodies (TS2/16, 9EG7, and 12G10), anti-α₅ antibody (SNAKA51), or PMA is required to promote cell adhesion (Clark K et al., J Cell Sci. 118:291-300 (2005)). Tensin induction by resveratrol also increased K562 cell adhesion to fibronectin, cell spreading and actin polymerization (Rodrigue C M et al., Oncogene 24:3274-84 (2005)).

The present invention contemplates the use of erythroleukemic cells that are transfected with the adhesion, integrin nucleic acids including but not limited to the preferred BCAM/Lu gene (Hines P C et al. (2003) supra; Zennadi R et al. (2004) supra; Gauthier E, et al., (2005) supra) to induce expression of BCAM-1/Lu alone or together with any other molecules with affinity for tumor microvasculature or tumor cells. Integrins/adhesion or any other molecules with affinity for the tumor microvasculature are useful in the claimed invention.

The present invention contemplates that before administration to tumor bearing patients, the BCAM/Lu laminin-α5 and ICAM-4 receptors on sickle cells (to include nucleated sickle cell progenitors, non-nucleated sickle cells and all sickle variants) are upregulated with epinephrine, PMA or other adrenergic stimuli, forskolin, phosphodiesterase inhibitors, cAMP analogs, pertussis toxin, okadeic acid or any agent which upregulates cAMP levels in the SS or erythroleukemia cell or sickle variants. In vitro treatment conditions include exposure to epinephrine of 1-5×10⁻² uM/10⁸ erythrocytes for 1-15 minutes at 37° C. (see Udani, Zennadi, Hines and Gauthier supra for additional useful treatments and conditions for upregulation of adhesion molecules on SS and erythroleukemia cells and sickle variants). Likewise, erythroleukemia cells (or erythroleukemia cells transduced with nucleic acids encoding BCAM/LU) are stimulated with adrenergic agents and treated with stimulatory anti-β₁-integrin antibodies (TS2/16, 9EG7, and 12G10), anti-α₅ antibody (SNAKA51), or PMA by methods given in Rodrigue C M (2005) supra; Clark K (2005) supra, to upregulate expression of α5v1 integrins before administration to tumor bearing patients. These cells are administered using the doses and volumes identical to those of sickle erythrocyte precursors and sickle erythrocytes described above and in Example 3.

In an additional embodiment, nucleated erythroleukemia cells are transfected with SS β globin gene to induce expression of sickle hemoglobin SA by homologous recombination using techniques well established in the art (Bender M A et al., Mol Cell Biol 8: 1725-1735 (1988); Oh I H et al., Exp Hematol. 32:461-9 (2004); Stoeckert C J et al., Exp Hematol. 18:1164-70 (1990); Zhou S Z et al., Exp Hematol. 21: 928-933 (1993)). Stable transduction of CD34+ stem cells from sickle cell patients has been achieved using nucleic acids encoding normal β globin with more than 50% of the progeny expressing SA hemoglobin by homologous recombination (Wu, L C et al., Blood (2006)). 13 globin genes from patients with any other sickle variant or hemoglobinopathy as mentioned above e.g., hemoglobin SA, SC, SE, S-thalassemia etc. are useful. The transduced leukemic cells assume the properties of a typical SA erythrocyte including hemoglobin polymerization when deoxygenated and increased structural rigidity. These transfected erythroleukemia cells may also be transfected with nucleic acids encoding SS β globin integrated into a replication competent or incompetent oncolytic virus under the control of the HRE.

In the present invention, erythroleukemia cells from mammalian donors or established cells lines are useful. They are isolated from bone marrow or peripheral blood of patients with erythroleukemia by apheresis using methodology well established in the art. Established human erythroleukemia cell lines are also useful including but not limited to the K562 or ERY-1 (Hines P C et al., supra (2003); Zennadi R et al., supra (2004); Gauthier E, et al., (2005) supra; Ribadeau D A et al., Leuk Res. 12: 1329-39 (2004)).

In another embodiment, erythroleukemia cells and SS cells to include nucleated sickle cell progenitors, non-nucleated sickle cells and all sickle variants in the natural state are adrenergically-upregulated or transduced with integrin/adhesion nucleic acids and useful as carriers of tumoricidal agents. A particularly preferred construct is human erythroleukemia cells (exemplified by K562 cells) or murine MEL cells stably transfected with nucleic acids encoding BCAM/lu alone or together with ICAM-4 and/or α4β1 (Parsons S F et al. Blood 89: 4219-4225 (1997).

Erythroleukemia cells (optionally stably transduced with BCAM/lu or other molecules avid for tumors) may be transduced with plasmids or vectors encoding oncolytic viruses including man-made and modified or mutant oncolytic viruses, tumoricidal proteins, toxins, toxin antibody conjugates, therapeutic protein, antibodies and antibody fragments and hemolysins. These cells are administered to tumor bearing hosts wherein they bind to tumor or tumor neovasculature and release their tumoricidal contents.

Expression of BCAM/Lu in K562 erythroleukemia cells. The full-length BCAM/Lu cDNA construct is subcloned into the pBabe puro retroviral vector (kindly provided by Dr H. Land, ICRF, London UK). The pBabe puro retroviral vector containing the BCAM/lu is linearized by digestion with Sca I before transfection. K562 cells (5×10⁵) are directly transfected with the pBabe puro construct containing full-length BCAM/lu cDNA (25 mg) using the calcium phosphate technique and by electroporation (200V, 1,000 mF). Transfectants are cultured in Iscove's modified Eagle's medium, 10% fetal calf serum (FCS) for 48 hours then plated over 96-well culture plates in medium containing 3 mg/mL puromycin (Sigma, Poole, UK). Individual puromycin-resistant colonies are isolated and tested for expression of BCAM/lu with monoclonal antibodies and immunoblotting. These cells are administered to the host in volumes of 5 to 1000 cc over 30 minutes to 180 minutes.

The same erythroleukemia cells are transduced with any and all of the oncolytic viruses including but not limited to the alphavirus and adenovirus constructs, tumoricidal proteins/toxins/hemolysins/conjugate nucleic acids using vectors, constructs, promoters, enhancers including but not limited to the HRE and signal sequences described herein for mature SS cells and SS progenitors.

### Use of Sickle Erythroblasts, Erythrocytes, Erythroleukemia Cells and Sickle Cell Ghosts In Vivo

### Subjects

The subjects treated are preferably human subjects and any mammalian species in which treatment or prevention of cancer is desirable, particularly agricultural and domestic mammalian species.

### Administration

Suitable methodology for administration of sickle erythrocytes, erythroblasts, sickle variants and erythroleukemia cells transduced with the various plasmids, vectors, oncolytic viruses, tumoricidal transgenes, proteins, antibodies, enzymes of the claimed invention is parenteral infusion or injection in a manner similar to a conventional blood transfusion with delivery between 5-1000 ml of cells/hr via a secure intravenous catheter.

### Dose

An effective dose of sickle erythrocytes is administered to a subject in need thereof. A “therapeutically effective amount” is an amount of the therapeutic composition sufficient to produce a measurable response (e.g., a cytolytic response in a subject being treated). Actual dosage levels of active ingredients in the pharmaceutical compositions of the claimed compositions are varied so as to administer an amount that is effective to achieve the desired therapeutic response for a particular subject.

The potency of a therapeutic composition can vary, and therefore a “therapeutically effective” amount can vary. However, using the assay methods described herein below, one skilled in the art can readily assess the potency and efficacy of a candidate modulator of this presently claimed subject matter and adjust the therapeutic regimen accordingly.

One of ordinary skill in the art can tailor the dosages to an individual patient, taking into account the particular formulation, method of administration to be used with the composition, and tumor size considering patient height and weight, severity and stage of symptoms, and the presence of additional deleterious physical conditions. Such adjustments or variations as well as evaluation of when and how to make such adjustments or variations are well known to those of ordinary skill.

Toxicity is assessed using criteria set forth by the National Cancer Institute and is reasonably defined as any grade 4 toxicity or any grade 3 toxicity persisting more than 1 week. Dose is also modified to maximize anti-tumor or anti-angiogenic activity.

### Therapeutic Cell

Previously it has been shown that the administration of irradiated tumor cells transfected with and secreting superantigen SEB to mice with metastatic mammary carcinoma resulted in a significantly improved survival and reduction of metastatic colonies in the lung (Terman D S U.S. Pat. No. 6,221,351). In the present invention, tumor cells with a metastatic phenotype (expressing the major chemokine receptors for this phenotype) are transfected with nucleic acids encoding superantigens and co-transduced with the Sindbis virus or the adenovirus expressing the fusogenic membrane glycoprotein. Optionally, the tumor cells may also be loaded with protoporphyrin IX by pretreatment with delta amino levulinic acid for 2-5 hours. The cells are irradiated with visible light for 2-60 minutes as described herein and then administered parenterally in doses of 10⁴-10¹¹ to tumor bearing hosts 2-3 times weekly for 2-6 weeks. Survival in treated group is significantly greater than untreated controls or controls treated with mock-transfected tumor cells using methods well established in the art.

### Chemotherapeutic and Other Agents

Chemotherapeutic agents can be used before, together with or after parenteral/systemic-administration of sickle erythrocytes, their nucleated precursors, sickle hemoglobin variants, erythroleukemia cells to enhance the tumor-killing effect. The sickle erythrocytes are defined in Definitions on page 1 as mature sickled cells, their nucleated precursors, sickle hemoglobin variants and erythroleukemia cells. These cells in native form or transduced with viral vectors/transgenes or upregulated with adrenergic agents are delivered by injection, instillation or infusion by any route including intravenously, intramuscularly, intradermally, intravesicularly, intrathecally, intrapleurally, intrapericardially, subcutaneously, intraperitoneally, and any other parenteral route. Chemotherapy is administered by infusion, instillation or injection by any parenteral route such as intrathecally, intratumorally, intravenously, intratumorally, intramuscularly, intradermally, intravesicularly, intrathecally, intrapleurally, intrapericardially, subcutaneously, intraperitoneally concomitantly with sickle erythrocyte. Preferably chemotherapy is given together with, before or 1-12 days after sickled erythrocytes, including native transgenes and their homologues, fragments, fusion proteins or mixtures thereof alone. Anti-cancer chemotherapeutic drugs useful in this invention include but are not limited to antimetabolites, anthracycline, vinca alkaloid, anti-tubulin drugs, antibiotics and alkylating agents. Representative specific drugs that can be used alone or in combination include cisplatinum (CDDP), adriamycin, dactinomycin, mitomycin, caminomycin, daunomycin, doxorubicin, tamoxifen, taxol, taxotere, vincristine, vinblastine, vinorelbine, etoposide (VP-16), 5-fluorouracil (5FU), cytosine arabinoside, cyclophosphamide, thiotepa, methotrexate, camptothecin, actinomycin-D, mitomycin C, aminopterin, combretastatin(s) and derivatives and prodrugs thereof.

A variety of chemotherapeutic and pharmacological agents may be given separately. Those of ordinary skill in the art will know how to select appropriate agents and doses, although, as disclosed, the doses of chemotherapeutic drugs are preferably reduced when used in combination with sickle erythrocyte in the present invention.

Another newer class of drugs that are also termed “chemotherapeutic agents” comprises agents that induce apoptosis. Any one or more of such drugs, including genes, vectors, antisense constructs, siRNA constructs, and ribozymes, as appropriate, may be used in conjunction with sickle erythrocytes.

Other agents useful herein are anti-angiogenic agents, such as Avastin, angiostatin, endostatin, vasculostatin, canstatin and maspin. Avastin or Bevacizumab is a recombinant humanized monoclonal antibody directed against vascular endothelial growth factor (VEGF). Human VEGF mediates neo-angiogenesis in normal and malignant vasculature. It is overexpressed in most malignancies, and high levels have correlated with a greater risk of metastasis. Avastin or bevacizumab binds VEGF and prevents its interaction with receptors (Flt-1 and KDR) on the surface of endothelial cells. Avastin 5 mg/kg intravenously is given every 14 days until disease progression is detected. The initial dose of Avastin is delivered over 90 minutes as an IV infusion. sickle erythrocyte, preferably sickle erythrocyte, are administered before, during or after avastin and usually given once or twice weekly for up to 10 weeks.

Chemotherapeutic agents are administered as single agents or multidrug combinations, in full or reduced dosage per treatment cycle. They can be administered before, during or after intrathecal or intratumoral, intravesicular and parenteral sickle erythrocyte composition. In a preferred schedule, the chemotherapeutic agent is administered within 36 hours of the last of two to four treatments of sickle erythrocyte compositions administered intrathecally (intrapleurally) or intratumorally or intravenously.

The combined use of the preferred sickle erythrocyte compositions with low dose, single agent chemotherapeutic drugs is particularly preferred. Indeed, this synergy of sickle erythrocyte with chemotherapy allows the use of the more toxic superantigens in lower and subtoxic doses as a means of priming a tumor for killing by chemotherapy. The choice of chemotherapeutic drug in such combinations is determined by the nature of the underlying malignancy. For lung tumors, cisplatinum is preferred. For breast cancer, a microtubule inhibitor such as taxotere is the preferred. For malignant ascites due to gastrointestinal tumors, 5-FU is preferred. “Low dose” as used with a chemotherapeutic drug refers to the dose of single agents that is 10-95% below that of the approved dosage for that agent (by the U.S. Food and Drug Administration, FDA). If the regimen consists of combination chemotherapy, then each drug dose is reduced by the same percentage. A reduction of >50% of the FDA approved dosage is preferred although therapeutic effects are seen with dosages above or below this level, with minimal side effects.

Tumors that are treated with sickle erythrocytes and chemotherapy are preferably at least 6 cm³ and visible by x-ray, CT, ultrasound, bronchoscopy, laparoscopy, culdoscopy. Localization of the agent delivered is facilitated with fluoroscopic, CT or ultrasound guidance. Representative tumors that are treatable with this approach include but are not limited to hepatocellular carcinoma, lung tumors, brain tumors, head and neck tumors and unresectable breast tumors. Multiple tumors at different sites may be treated by intrathecal or intratumoral chemotherapy and parenterally administered sickle erythrocytes.

The chemotherapeutic agent(s) selected for therapy of a particular tumor preferably is one with the highest response rates against that type of tumor. For example, for non-small cell lung cancer (NSCLC), cisplatinum-based drugs have been proven effective. Cisplatinum may be given parenterally or intratumorally. When given intratumorally, cisplatinum is preferentially in small volume around 1-4 ml although larger volumes can also work. The smaller volume is designed to increase the viscosity of the cisplatinum containing solution in order to minimize or delay the clearance of the drug from the tumor site. Other agents useful in NSCLC include the taxanes (paclitaxel and docetaxel), vinca alkaloids (vinorelbine), antimetabolites (gemcitabine), and camptothecin (irinotecan) both as single agents and in combination with a platinum agent.

The optimal chemotherapeutic agents and combined regimens for all the major human tumors are set forth in Bethesda Handbook of Clinical Oncology, Abraham J et al., Lippincott William & Wilkins, Philadelphia, Pa. (2001); Manual of Clinical Oncology, Fourth Edition, Casciato, D A et al., Lippincott William & Wilkins, Philadelphia, Pa. (2000) both of which are herein incorporated in entirety by reference.

In one embodiment, these recommended chemotherapeutic agents are used alone or combined with other chemotherapeutics in subtherapeutic or full doses. Alternatively, they may be administered parenterally by infusion, instillation or injection in doses 10-95% below the FDA recommended therapeutic dose. For intratumoral administration, the dose of a chemotherapeutic drug or biologic agent is preferably reduced 10- to 50-fold below the FDA-recommended dose for parenteral administration. Chemotherapy in full or reduced dose can be administered parenterally by injection, instillation or infusion parenterally by any route such as intrathecally, intratumorally intravenously, intramuscularly, intradermally, intravesicularly, intrathecally, intrapleurally, intrapericardially, subcutaneously, intraperitoneally concomitant with, before or after the SAg.

Cisplatinum has been widely used to treat cancer, with effective parenteral doses of 20 mg/m² for 5 days every three weeks for a total of three courses. Preferred dose per treatment for cisplatinum given intratumorally is 5-10 mg whereas for intrathecal use 20-80 mg may be administered. Intratumoral cisplatinum may be given every 7-14 days for 10-20 treatments whereas intrathecal cisplatinum may be given every 2-6 weeks for 10-20 treatments. Cisplatinum delivered in small volumes, e.g., 5-10 mg/1-3 ml saline is extremely viscous and may be retained in the tumor for a sustained period acting much like a controlled release drug from an inert surface. This is indeed one preferred mode of administration of cisplatinum when administered intratumorally with or without the superantigen.

When used before, together with or after sickle erythrocyte administration, doses of chemotherapy are used preferably in full doses but may be reduced 10-95% below the FDA recommended therapeutic dose. For intratumoral administration, the dose of a chemotherapeutic drug or biologic agent may be reduced 10- to 50-fold below the FDA-recommended dose for parenteral administration. Cisplatinum is preferably given systemically with effective doses of 20 mg/m² for 5 days every three weeks for a total of three courses. For intratumoral use a cisplatinum dose of is 5-50 mg/lesion is given whereas for intrathecal use 20-80 mg may be administered. Intratumoral cisplatinum may be given every 7-14 days for 10-20 treatments whereas intrathecal cisplatinum may be given every 2-6 weeks for 10-20 treatments. Cisplatinum delivered in small volumes, e.g., 5-10 mg/1-3 ml saline is extremely viscous and may be retained in the tumor for a sustained period acting much like a controlled release drug from an inert surface. However the cisplatinum or chemotherapy is also effective when given in non-viscous form before, together with or after egc SAg therapy.

Other agents and therapies that are useful together with or after parenteral (e.g., intratumoral, intrapleural, intraperitoneal, intravesicular, intravenous) sickle erythrocytes include, radiotherapeutic agents, antitumor antibodies with attached anti-tumor drugs such as plant-, fungus-, or bacteria-derived toxin or coagulant, ricin A chain, deglycosylated ricin A chain, ribosome inactivating proteins, sarcins, gelonin, aspergillin, restrictocin, a ribonuclease, a epipodophyllotoxin, diphtheria toxin, or Pseudomonas exotoxin. Additional cytotoxic, cytostatic or anti-cellular agents capable of killing or suppressing the growth or division of tumor cells include anti-angiogenic agents, interferons alpha and gamma, apoptosis-inducing agents, coagulants, prodrugs or tumor targeted forms, tyrosine kinase inhibitors (Siemeister et al., Cancer Metastasis Rev. 17:241-8 (1998), antisense strategies, RNA aptamers, siRNA and ribozymes against VEGF or VEGF receptors (Saleh M et al., Cancer Res. 56:393-401 (1996); Cheng et al., Proc Natl Acad Sci 93:8502-7 (1996); Ke et al., Int J. Oncol. 12:1391-6 (1998); Parry et al., Antisense Nucleic Acid Drug Dev. 9:271-7 (1999)); each incorporated herein by reference.

Any of a number of tyrosine kinase inhibitors is useful when administered before, together with, or after, intratumoral sickle erythrocytes. These include, for example, the 4-aminopyrrolo[2,3-d]pyrimidines (U.S. Pat. No. 5,639,757). Further examples of small organic molecules capable of modulating tyrosine kinase signal transduction via the VEGF-R2 receptor are the quinazoline compounds and compositions (U.S. Pat. No. 5,792,771). Tarceva or Erlotinib attaches to EGF receptors and thereby blocks the EGF-mediated activation of tyrosine kinase. Tarceva 150 mg daily is administered before during or after parenteral (intrathecal, intrapleural and/or intravenous) sickle erythrocyte treatment and continued until disease progression or unacceptable toxicity occurs.

Other agents which may be employed in combination with sickle erythrocytes are steroids such as the angiostatic 4,9(11)-steroids and C21-oxygenated steroids (U.S. Pat. No. 5,972,922). Thalidomide and related compounds, precursors, analogs, metabolites and hydrolysis products (U.S. Pat. Nos. 5,712,291 and 5,593,990) may also be used in combination with SAgs and other chemotherapeutic drugs agents to inhibit angiogenesis. These thalidomide and related compounds can be administered orally.

Certain anti-angiogenic agents that cause tumor regression may be administered before, together with, or after, intrathecal, intrapleural, intratumoral, intravenous or parenteral sickle erythrocytes. These include the bacterial polysaccharide CM101 (currently in clinical trials as an anti-cancer drug) and the antibody LM609. CM101 has been well characterized for its ability to induce neovascular inflammation in tumors. CM101 binds to and cross-links receptors expressed on dedifferentiated endothelium that stimulate the activation of the complement system. It also initiates a cytokine-driven inflammatory response that selectively targets the tumor. CM101 is a uniquely antiangiogenic agent that downregulates the expression VEGF and its receptors. Thrombospondin (TSP-1) and platelet factor 4 (PF4) may also be used together with or after intratumoral SAg. These are both angiogenesis inhibitors that associate with heparin and are found in platelet α granules.

Interferons and metalloproteinase inhibitors are two other classes of naturally occurring angiogenic inhibitors that can be used before, together with or after intratumoral SAg. Vascular tumors in particular are sensitive to interferon; for example, proliferating hemangiomas are successfully treated with IFNα. Tissue inhibitors of metalloproteinases (TIMPs), a family of naturally occurring inhibitors of matrix metalloproteases (MMPs), can also inhibit angiogenesis and can be used in combination (before, during or after) the SAgs.

### Radiation Therapy

Local radiation to any tumor sites or the mediastinum using the traditional standard dose of 60-65 gy is given concomitant with parenteral (e.g., intrathecal, intravenous, intravesicular, intrapleural, intralymphatic or intratumoral) administration of sickled erythrocytes. The radiotherapy is also be given before, during or after the sickled erythrocyte therapy but in either case there is a hiatus of no more than 30 days between the start of sickled erythrocyte therapy and the start or conclusion of radiotherapy. The median survival of patients given this type of radiotherapy alone is 5% at one year whereas the combined modality improves the median survival to more than two years.

In general, local radiation therapy alone has minimal efficacy in contributing to long-term disease control in advanced carcinomas. While radiation is an effective palliative measure to relieve symptoms, only a very small minority of patients achieve long-term survival when treated with radiation alone. However, radiation synergizes with sickle erythrocyte therapy in shrinking tumors and prolonging survival. Radiation is given to bulky or symptomatic lung lesions before, during or after sickle erythrocyte therapy. Preferably it is started 1-2 weeks before sickle erythrocyte treatment and continued simultaneously with sickle erythrocyte for 1-4 weeks until the full courses of sickle erythrocyte and radiation are completed. It may also be started after sickle erythrocyte treatment preferably within 24 hours of the last sickle erythrocyte treatment. Radiation may also be given to a malignant lesion or a tumorous body cavity before, together with or after the site has been injected with sickle erythrocyte intratumorally or intrathecally and/or system ic/parenteral chemotherapy. It may also be administered to a malignant lesion or site not injected specifically with sickle erythrocytes. In this case the sickle erythrocyte may be given systemically or intrathecally but not directly to the radiated tumor mass or site. Regimens for the use of intratumoral sickle erythrocytes and intratumoral and/or systemic use of chemotherapy are described in previous sections on chemotherapy. Radiation may also be used with chemotherapy in these settings together with systemic and/or intratumoral sickle erythrocyte treatment and intratumoral or systemic chemotherapy.

Radiation techniques are preferably continuous rather than split. Hyper-fractionated radiation, employing multiple daily fractions of radiation is preferred to conventionally fractionated radiation. Radiation doses vary from 40-70 gy although a dose between 60 and 70 gy dose is preferred. It is contemplated that radiation doses considered being subtherapeutic and up to 70% below the conventional doses are also useful when used before, during or after a course of sickle erythrocyte therapy.

### Production and Isolation of Superantigens

The superantigens disclosed herein are prepared by either biochemical isolation, or, preferably by recombinant methods. The following SAgs, including their sequences and biological activities have been known for a number of years. Studies of these SAgs are found throughout the biomedical literature. For, biochemical and recombinant preparation of these SAgs see the following references: Borst, D W et al., Infect. Immun. 61: 5421-5425 (1993); Couch, J L et al., J. Bacteriol. 170: 2954-2960 (1988); Jones, C L et al., J. Bacteriol. 166: 29-33 (1986); Bayles K W et al., J. Bacteriol. 171: 4799-4806 (1989); Blomster-Hautamaa, D A et al., J. Biol. Chem. 261:15783-15786 (1986); Johnson, L P et al., Mol. Gen. Genet. 203, 354-356 (1986); Bohach G A et al., Infect. Immun. 55: 428-433 (1987); Iandolo J J et al., Meth. Enzymol 165:43-52 (1988); Spero L et al., Meth. Enzymol 78(Pt A):331-6 (1981); Blomster-Hautamaa D A, Meth. Enzymol 165: 37-43 (1988); Iandolo J J Ann. Rev. Microbiol. 43: 375-402 (1989); U.S. Pat. No. 6,126,945 and U.S. provisional patent application 60/389,366 filed Jun. 15, 2002. These references and the references cited therein are hereby incorporated by reference in their entirety.

These SAgs are Staphylococcal enterotoxin A (SEA), Staphylococcal enterotoxin B (SEB), Staphylococcal enterotoxin C (SEC—actually three different proteins, SEC1, SEC2 and SEC3)), Staphylococcal enterotoxin D (SED), Staphylococcal enterotoxin E (SEE) and toxic shock syndrome toxin-1 (TSST-1) (U.S. Pat. No. 6,126,945 and U.S. provisional patent application 60/389,366 filed Jun. 15, 2002, and the references cited therein). The amino acids sequences of the above group of native (wild-type) SAgs is provided below:

| SEA (Huang, I. Y. et al., J. Biol. Chem. 262: | |
|---|---|
| 7006-7013 (1987)) | |
| [SEQ ID NO: 1] | |
| 1 | SEKSEEINEK DLRKKSELQG TAGNKQIY YYNEKAKTEN KESHDQFLQH |
| | TILFKGFFTD |
| 61 | HSWYNDLLVD FDSKDIVDKY KGKKVDLYGA YYGYQCAGGT PNKTACMYGG |
| | VTLHDNNRLT |
| 121 | EEKKVPINLW LDGKQNTVPL ETVKTNKKNV TVQELDLQAR RYLQEKYNLY |
| | NSDVFDGKVQ |
| 181 | RGLIVFHTST EPSVNYDLFG AQGQYSNTLL RIYRDNKSIN SENMHIDIYL YTS |
| SEB (Papageorgiou, A. C. et al. J. Mol. Biol. 277: | |
| 61-79 (1998)) | |
| [SEQ ID NO: 2] | |
| 1 | ESQPDPKPDE LHKSSKFTGL MENMKVLYDD NHVSAINVKS IDQFLYFDLI |
| | YSIKDTKLGN |
| 61 | YDNVRVEFKN KDLADKYKDK YVDVFGANYY YQCYFSKKTN DINSHQTDKR |
| | KTCMYGGVTE |
| 121 | HNGNQLDKYR SITVRVFEDG KNLLSFDVQT NKKKVTAQEL DYLTRHYLVK |
| | NKKLYEFNNS |
| 181 | PYETGYIKFI ENENSFWYDM MPAPGDKFDQ SKYLMMYNDN KMVDSKDVKI |
| | EVYLTTKK |
| SEC1 (Bohach, G. A. et al., Mol. Gen. Genet. 209: | |
| 15-20 (1987)) | |
| [SEQ ID NO: 3] | |
| 1 | MNKSRFISCV ILIFALILVL FTPNVLAESQ PDPTPDELHK ASKFTGLMEN |
| | MKVLYDDHYV |
| 61 | SATKVKSVDK FLAHDLIYNI SDKKLKNYDK VKTELLNEGL AKKYKDEVVD |
| | VYGSNYYVNC |
| 121 | YFSSKDNVGK VTGGKTCMYG GITKHEGNHF DNGNLQNVLI RVYENKRNTI |
| | SFEVQTDKKS |
| 181 | VTAQELDIKA RNFLINKKNL YEFNSSPYET GYIKFIENNG NTFWYDMMPA |
| | PGDKFDQSKY |
| SEC2 (Papageorgiou, A. C., et al., Structure 3: | |
| 769-779 (1995)) | |
| [SEQ ID NO: 4] | |
| 1 | ESQPDPTPDE LHKSSEFTGT MGNMKYLYDD HYVSATKVMS VDKFLAHDLI |
| | YNISDKKLKN |
| 61 | YDKVKTELLN EDLAKKYKDE VVDVYGSNYY VNCYFSSKDN VGKVTGGKTC |
| | MYGGITKHEG |
| 121 | NHFDNGNLQN VLIRVYENKR NTISFEVQTD KKSVTAQELD IKARNFLINK |
| | KNLYEFNSSP |
| 181 | YETGYIKFIE NNGNTFWYDM MPAPGDKFDQ SKYLMMYNDN KTVDSKSVKI |
| | EVHLTTKNG |
| SEC3 (Hovde, C. J. et al., Mol. Gen. Genet. 220: | |
| 329-333 (1990)) | |
| [SEQ ID NO: 5] | |
| 1 | MYKRLFISRV ILIFALILVI STPNVLAESQ PDPMPDDLHK SSEFTGTMGN |
| | MKYLYDDHYV |
| 61 | SATKVKSVDK FLAHDLIYNI SDKKLKNYDK VKTELLNEDL AKKYKDEVVD |
| | VYGSNYYVNC |
| 121 | YFSSKDNVGK VTGGKTCMYG GITKHEGNHF DNGNLQNVLV RVYENKRNTI |
| | SFEVQTDKKS |
| 181 | VTAQELDIKA RNFLINKKNL YEFNSSPYET GYIKFIENNG NTFWYDMMPA |
| | PGDKFDQSKY |
| 241 | LMMYNDNKTV DSKSVKIEVH LTTKNG |
| SED (Bayles, K. W. et al., J. Bacteriol. 171: | |
| 4799-4806 (1989)) | |
| [SEQ ID NO: 6] | |
| 1 | MKKFNILIAL LFFTSLVISP LNVKANENID SVKEKELHKK SELSSTALNN |
| | MKHSYADKNP |
| 61 | IIGENKSTGD QFLENTLLYK KFFTDLINFE DLLINFNSKE MAQHFKSKNV |
| | DVYPIRYSIN |
| 121 | CYGGEIDRTA CTYGGVTPHE GNKLKERKKI PINLWINGVQ KEVSLDKVQT |
| | DKKNVTVQEL |
| 181 | DAQARRYLQK DLKLYNNDTL GGKIQRGKIE FDSSDGSKVS YDLFDVKGDF |
| | PEKQLRIYSD |
| 241 | NKTLSTEHLH IDIYLYEK |
| SEE (Couch, J. L. et al., J. Bacteriol. 170: | |
| 2954-2960 (1988)) | |
| [SEQ ID NO: 7] | |
| 1 | MKKTAFILLL FIALTLTTSP LVNGSEKSEE INEKDLRKKS ELQRNALSNL |
| | RQIYYYNEKA |
| 61 | ITENKESDDQ FLENTLLFKG FFTGHPWYND LLVDLGSKDA TNKYKGKKVD |
| | LYGAYYGYQC |
| 121 | AGGTPNKTAC MYGGVTLHDN NRLTEEKKVP INLWIDGKQT TVPIDKVKTS |
| | KKEVTVQELD |
| 181 | LQARHYLHGK FGLYNSDSFG GKVQRGLIVF HSSEGSTVSY DLFDAQGQYP |
| | DTLLRIYRDN |
| 241 | KTINSENLHI DLYLYTT |
| TSST-1 (Prasad, G. S. et al., Protein Sci. 6: | |
| 1220-1227 (1997)) | |
| [SEQ ID NO: 8] | |
| 1 | MNKKLLMNFF IVSPLLLATT ATDFTPVPLS SNQIIKTAKA STNDNIKDLL |
| | DWYSSGSDTF |
| 61 | TNSEVLDNSL GSMRIKNTDG SISLIIFPSP YYSPAFTKGE KVDLNTKRTK |
| | KSQHTSEGTY |
| 121 | IHFQISGVTN TEKLPTPIEL PLKVKVHGKD SPLKYGPKFD KKQLAISTLD |
| | FEIRHQLTQI |
| 181 | HGLYRSSDKT GGYWKITMND GSTYQSDLSK KFEYNTEKPP INIDEIKTIE AEIN |

The sections which follow discuss SAgs which have been discovered and characterized more recently.

### Staphylococcal Enterotoxins SEG, SEH, SEI, SEJ, SEK, SEL, SEM, SEN, SEO, SEP, SEQ, SER, SEU

New Staphylococcal enterotoxins G, H, I, J, K, L and M (SEG, SEH, SEI, SEJ, SEK, SEL, SEM, SEN, SEO, SEP, SEQ, SER, SEU; abbreviated below as “SEG-SEU”) were described in Jarraud, S. et al., J. Immunol. 166: 669-677 (2001); Jarraud S et al., J. Clin. Microbiol. 37: 2446-2449 (1999) and Munson, S H et al., Infect. Immun. 66:3337-3345 (1998). SEG-SEU show superantigenic activity and are capable of inducing tumoricidal effects. The homology of these SE's to the better known SE's in the family ranges from 27-64%. Each induces selective expansion of TCR Vβ subsets. Thus, these SEs retain the characteristics of T cell activation and Vβ usage common to all the other SE's. RT-PCR was used to show that SEH stimulates human T cells via the Vα domain of TCR, in particular Vα (TRAV27), while no TCR Vβ-specific expansion was seen. This is in sharp contrast to all other studied bacterial superantigens, which are highly specific for TCR Vβ. Vβ binding superantigens form one group, whereas SEH has different properties that fit well with Vα reactivity. It is suggested that SEH directly interacts with the TCR Vα domain (Petersson K et al., J Immunol. 170:4148-54 (2003)).

SEG and SEH of this group and other enterotoxins including SPEA, SPEC, SPEG, SPEH, SME-Z, SME-Z2, (see below) utilize zinc as part of high affinity MHC class II receptor. Amino acid substitution(s) at the high-affinity, zinc-dependent class II binding site are created to reduce their affinity for MHC class II molecules.

Jarraud S et al., 2001, supra, discloses methods used to identify and characterize egc SEs SEG-SEM, and for cloning and recombinant expression of these proteins. The egc comprises SEG, SEI, SEM, SEN, SEO and pseudogene products designated ψent 1 and ψent 2. Purified recombinant SEN, SEM, SEI, SEQ, and SEGL29P (a mutant of SEN) were expressed in E. coli. Recombinant SEG, SEN, SEM, SEI, and SEQ consistently induced selective expansion of distinct subpopulations of T cells expressing particular VP genes.

Jarraud S et al., 2001, supra, indicates that the seven genes and pseudogenes composing the egc (enterotoxin gene cluster) operon are co-transcribed. The association of related co-transcribed genes suggested that the resulting peptides might have complementary effects on the host's immune response. One hypothesis is that gene recombination created new SE variants differing by their superantigen activity profiles. By contrast, SEGL29P failed to trigger expansion of any of 23 Vβ subsets, and the L29P mutation accounted for the complete loss of superantigen activity (although this mutation did not induce a major conformational change). It is believed that this substitution mutation located at a position crucial for proper superantigen/MHC II interaction.

Overall, TCR repertoire analysis confirms the superantigenic nature of SEG, SEI, SEM, SEN, SEO. These investigators used a number of TCR-specific mAbs (Vβ specificity indicated in brackets) for flow cytometric analysis: E2.2E7.2 (Vβ2), LE89 (Vβ3), IMMU157 (Vβ5.1), 3D11 (Vβ5.3), CR1304.3 (Vβ6.2), 3G5D15 (Vβ7), 56C5.2 (Vβ8.1/8.2), FIN9 (Vβ9), C21 (Vβ11), S511 (Vβ12), IMMU1222 (Vβ13.1), JIJ74 (Vβ13.6), CAS1.1.13 (Vβ14), Tamaya1.2 (Vβ16), E17.5F3 (Vβ17), βA62.6 (Vβ18), ELL1.4 (Vβ20), IG125 (Vβ21.3), IMMU546 (Vβ22), and HUT78.1 (Vβ23). Flow cytometry also revealed preferential expansion of CD4+ T cells in SEI and SEM cultures. By contrast, the CD4/CD8 ratios in SEQ-, SEN-, and SEG-stimulated T cell lines were close to those in fresh PBL.

Recombinant and biochemical preparation of the egc SEs is given in U.S. 60/799,514, PCTUS05/022638, U.S. 60/583,692, U.S. 60/665,654, U.S. 60/626,159 which incorporated by reference and their references in their entirety.

The amino acid sequences of SEG-SEU are shown below

| SEG (Baba, T. et al., Lancet 359, 1819-1827 (2002)) | |
|---|---|
| [SEQ ID NO: 9] | |
| 1 | MNKIFRVLTV SLFFFTFLIK NNLAYADVGV INLRNFYANY QPEKLQGVSS |
| | GNFSTSHQLE |
| 61 | YIDGKYTLYS QFHNEYEAKR LKDHKVDIFG ISYSGLCNTK YMYGGITLAN |
| | QNLDKPRNIP |
| 121 | INLWVNGKQN TISTDKVSTQ KKEVTAQEID IKLRKYLQNE YNIYGFNKTK |
| | KGQEYGYKSK |
| 181 | FNSGFNKGKI TFHLNNEPSF TYDLFYTGTG QAESFLKIYN DNKTIDAENF |
| | HLDVEISYEK |
| 241 | TE |
| SEG (Jarraud, S et al., J. Immunol. 166: | |
| 669-677 (2001)) | |
| (SEQ ID NO: 10) | |
| 1 | MKKLSTVIII LILEIVFHNM NYVNAQPDLK LDELNKVSDK NNKGTMGNVM |
| | NLYTSPPVEG |
| 61 | RGVINSRQFL SHDLIFPIEY KSYNEVKTEL ELENTELANN YKDKKVDIFG |
| | VPYFYTCIIP |
| 121 | KSEPDINQNF GGCCMYGGLT FNSSENERDK LIYVQVTIDN RQSLGFTITT |
| | NKNMVTIQEL |
| 181 | DYKARHWTKE KKLYEFDGSA FESGYIKFTE KNNTSFWFDL FPKKELVPFV |
| | PYKFLNIYGD |
| 241 | NKVVDSKSIK MEVFLNTH |
| SEH (Omoe, K. et al., J. Clin. Microbiol. 40: | |
| 857-862 (2002)) | |
| [SEQ ID NO: 11] | |
| 1 | EDLHDKSELT DLALANAYGQ YNHPFIKENI KSDEISGEKD LIFRNQGDSG |
| | NDLRVKFATA |
| 61 | DLAQKFKNKN VDIYGASFYY KCEKISENIS ECLYGGTTLN SEKLAQERVI |
| | GANVWVDGIQ |
| 121 | KETELIRTNK KNVTLQELDI KIRKILSDKY KIYYKDSEIS KGLIEFDMKT |
| | PRDYSFDIYD |
| 181 | LKGENDYEID KIYEDNKTLK SDDISHIDVN LYTKKKV |
| SEI (Kuroda, M. et al., Lancet 357 (9264), | |
| 1225-1240 (2001)) | |
| [SEQ ID NO: 12] | |
| 1 | MKKFKYSFIL VFILLFNIKD LTYAQGDIGV GNLRNFYTKH DYIDLKGVTD |
| | KNLPIANQLE |
| 61 | FSTGTNDLIS ESNNWDEISK FKGKKLDIFG IDYNGPCKSK YMYGGATLSG |
| | QYLNSARKIP |
| 121 | INLWVNGKHK TISTDKIATN KKLVTAQEID VKLRRYLQEE YNIYGHNNTG |
| | KGKEYGYKSK |
| 181 | FYSGFNNGKV LFHLNNEKSF SYDLFYTGDG LPVSFLKIYE DNKIIESEKF |
| | HLDVEISYVD |
| 241 | SN |
| SEJ (Zhang, S. et al., FEMS Microbiol. Lett. | |
| 168: 227-233 (1998)) | |
| [SEQ ID NO: 13] | |
| 1 | MKKTIFILIF SLTLTLLITP LVYSDSKNET IKEKNLHKKS ELSSITLNNL |
| | RHIYFFNEKG |
| 61 | ISEKIMTEDQ FLDYTLLFKS FFISHSQYND LLVQFDSKET VNKFKGKQVD |
| | LYGSYYGFQC |
| 121 | SGGKPNKTAC MYGGVTLHEN NQLYDTKKIP INLWIDSIRT VVPLDIVKTN |
| | KKKVTIQELD |
| 181 | LQARYYLHKQ YNLYNPSTFD GKIQKGLIVF HTSKEPLVSY DLFNVIGQYP |
| | DKLLKIYQDN |
| 241 | KIIESENMHI DIYLYTSLIV LISLPLVL |
| SEK (Baba, T., et al., Lancet 359: | |
| 1819-1827 (2002)) | |
| [SEQ ID NO: 14] | |
| 1 | MKKLISILLI NIIILGVSNN ASAQGDIGID NLRNFYTKKD FINLKDVKDN |
| | DTPIANQLQF |
| 61 | SNESYDLISE SKDFNKFSNF KGKKLDVFGI SYNGQCNTKY IYGGITATNE |
| | YLDKPRNIPI |
| 121 | NIWINGNHKT ISTNKVSTNK KFVTAQEIDI KLRRYLQEEY NIYGHNGTKK |
| | GEEYGHKSKF |
| 181 | YSGFNIGKVT FHLNNNDTFS YDLFYTGDDG LPKSFLKIYE DNKTVESEKF |
| | HLDVDISYKE |
| 241 | TK |
| SEL (Kuroda, M. et al., Lancet 357: | |
| 1225-1240 (2001)) | |
| [SEQ ID NO: 15] | |
| 1 | MKKRLLFVIV ITLFIFSSNH TVLSNGDVGP GNLRNFYTKY EYVNLKNVKD |
| | KNSPESHRLE |
| 61 | YSYKNDTLYA EFDNEYITSD LKGKNVDVFG ISYKYGSNSR TIYGGVTKAE |
| | NNKLDSPRII |
| 121 | PINLIINGKH QTVTTKSVST DKKMVTAQEI DVKLRKYLQD EFNIYGHNDT |
| | GKGKEYGTSS |
| 181 | KFYSGFDKGS VVFHMNDGSN FSYDLFYTGY GLPESFLKIY KDNKTVDSTQ |
| | FHLDVEISKR |
| SEM (Kuroda, M. et al., Lancet 357: | |
| 1225-1240 (2001)) | |
| [SEQ ID NO: 16] | |
| 1 | MKRILIIVVL LFCYSQNHIA TADVGVLNLR NYYGSYPIED HQSINPENNH |
| | LSHQLVFSMD |
| 61 | NSTVTAEFKN VDDVKKFKNH AVDVYGLSYS GYCLKNKYIY GGVTLAGDYL |
| | EKSRRIPINL |
| 121 | WVNGEHQTIS TDKVSTNKKL VTAQEIDTKL RRYLQEEYNI YGFNDTNKGR |
| | NYGNKSKFSS |
| 181 | GFNAGKILFH LNDGSSFSYD LFDTGTGQAE SFLKIYNDNK TVETEKFHLD |
| | VEISYKDES |
| SEN (Jarraud, S et al., J. Immunol. 166: | |
| 669-677 (2001)) | |
| (SEQ ID NO: 17) | |
| 1 | MKNSKVMLNV LLLILNLIAI CSVNNAYANE EDPKIESLCK KSSVGPIALH |
| | NINDDYINNR |
| 61 | RFTTVKSIVS TTEKFLDFDL LFKSINWLDG ISAEFKDLKE |
| | FSSSAISKEF LGKYVDIYGV |
| 121 | YYKAHCHGEH QVDTACTYGG VTPHENNKLS EPKNIGVAVY KDNVNVNVNT |
| | FIVTTDKKK |
| 181 | VYAQELDIKV RTKLNNAYKL YDRMTSDVQK GYIKFHSHSE |
| | HKESFYYDLF YIKGNLPDQY |
| 241 | LQIYNDNKTT IDSSDYHIDV YLFT |
| SEO (Jarraud, S et al., J. Immunol. 166: | |
| 669-677 (2001)) | |
| (SEQ ID NO: 18) | |
| 1 | MKNIKKLMRL FYIAAIIITL LCLINNNYVN AEVDKKDLKK KSDLDSSKLFN |
| | LTSYYTDITW |
| 61 | QLDESNKIST DQLNNYIILK NIDISVLKTS SLKVEFNSSD LANQFKGKNUD |
| | IYGLYFGNKC |
| 121 | VGLTEEKTSC LYGGVTIHDG NQLDEEKVIG VNGFKDGVQQ EGFVIKTKKAK |
| | VTVQELDTKV |
| 181 | RFKLENLYKI YNKDTGNIQK GCIFFHSHNH QDQSFYYDLY NVKGSVGAEFF |
| | QFYSDNRTVS |
| 241 | SSNYHIDVFL YKD |
| ψent 1 (Jarraud, S et al., J. Immunol. 166: | |
| 669-677 (2001)) | |
| (SEQ ID NO: 19) | |
| 1 | MKLFAFIFIC VKSCSLLFML NGNPKPEQLN KASEFTGLMD NMRYLYDDKH |
| | VSETNIKSQE |
| 61 | KFLQHDLLFK INGSKILKTE FNNKSLSDKY KNKNVDLFGT NYYNQCYFSL |
| | DNMELNDGRL |
| 121 | IEKNVYVWRC GL |
| ψent 2 (Jarraud, S et al., J. Immunol. 166: | |
| 669-677 (2001)) | |
| (SEQ ID NO: 20) | |
| 1 | MYGGVVYENE RNSLSFDIPT NKKNITAQEI DYKVRNYLLK HKNLYEFNSSP |
| | YETGYIKFIE |
| 61 | GSGHSFWYDL MPESGKKFYP TKYLLIYNDN KTVESKSINV EVHLTKK |
| SEP (Kuroda, M. et al., Lancet 357, | |
| 1225-1240 (2001)) | |
| [SEQ ID NO: 21] | |
| 1 | MSKMKKTAFT LLLFIALTLT TSPLVNGSEK SEEINEKDLR KKSELQGTAL |
| | GNLKQIYYN |
| 61 | EKAKTENKES HDQFLQHTIL FKGFFTDHSW YNDLLVDFDS KDIVDKYKGK |
| | KVDLYFAYYG |
| 121 | YQCAGGTPNK TACMYGGVTL HDNNRLTEEK KEPINLWLDG KQNTVPLETV |
| | KTNKKVTVQ |
| 181 | ELDLQARRYL QEKYNLYNSD VFDGKVQRGL IVFHTSTEPS VNYDLFGAQG |
| | QYSNTLLRIY |
| 241 | RDNKTINSEN MHIDIYLYTS |
| SEQ (Lindsay, JA et al., Mol. Microbiol. 29, | |
| 527-543 (1998)) | |
| [SEQ ID NO: 22] | |
| 1 | MPIWRCNIKK GAIKMNKIFR ILTVSLFFFT FLIKNNLAYA DVGVINLRNF |
| | YANYEPEKLQ |
| 61 | GVSSGNFSTS HQLEYIDGKY TLYSQFHNEY EAKRLKDHKV DIFGISYSGL |
| | CNTKYMGGI |
| 121 | TLANQNLDKP RNIPINLWVN GKQNTISTDK VSTQKKEVTA QEIDIKLRKY |
| | LQNEYNIYGF |
| 181 | NKTKKGGEYG YQSKFNSGFN KGKITFHLNN EPSFTYDLFY TGTGGAESFL |
| | KIYNDNKTID |
| 241 | AENFHLDVEI SYEKTE |
| SER Omoe, K et al., ACCESSION BAC97795 | |
| [SEQ ID NO: 23] | |
| 1 | MLNKILLLLF SVTFMLLFFS LHSVSAKPDP RPGELNRVSD YKKNKGTMGN |
| | VESLYKDKAV |
| 61 | IAENVKNTRQ FLGHDLIFPI PYSEYKEVKS EFINKKTADK FKDKRLDVFG |
| | IPYFYTCLVP |
| 121 | KNESREEFIF DGVCIYGGVT MHSTADSISK NIIVPVTVDN KQQFSFTIST |
| | NKKTVTVQEL |
| 181 | DYKVRNWLTN NKKLYEFDGS AYETGYIKFI EQNKDSFWYD LFPKKDLVPF |
| | IPYKFVNIYG |
| 241 | DNKTIDASSV KIEVHLTTM |
| SEU (Letertre, C et al., J. Appl. Microbiol. 95, | |
| 38-43 (2003)) | |
| [SEQ ID NO: 24] | |
| 1 | MKLFAFIFIC VKSCSLLFML NGNPRPEQLN KASEFSGLMD NMRYLYDDKH |
| | VSETNIKAQE |
| 61 | KFLQHDLLFK INGSKIDGSK ILKTEFNNKS LSDKYKNKNV DLFGTNYYNQ |
| | CYFSADNMEL |
| 121 | NDGRLIEKTC MYGGVTEHDG NQIDKNNLTD NSHNILIKVY ENERNTLSFD |
| | ISTNMKNITA |
| 181 | QEIDYKVRNY LLKHKNLYEF NSSPYESGYI KFIEGNGHSF WYDMMPESGE |
| | KFYPTKYLLI |
| 241 | YNDNKTVESK SINVEVHLTK K |

### Streptococcal Pyrogenic Exotoxins (SpEs)

The SpE's SPEA, SPEB, SPEC, SPEG, SPEH, SME-Z, SME-Z2 and SSA are superantigens induce tumoricidal effects. SPEA, SPEB, SPEC have been known for some time and their structures and biological activities described in numerous publications.

SPEG, SPEH, and SPEJ genes were identified from the Streptococcus pyogenes M1 genomic database and described in detail in Proft, T et al., J. Exp. Med. 189: 89-101 (1999) which also describes SMEZ, SMEZ-2. This document also describes the cloning and expression of the genes encoding these proteins.

The smez-2 gene was isolated from the S. pyogenes strain 2035, based on sequence homology to the streptococcal mitogenic exotoxin z (smez) gene. SMEZ-2, SPE-G, and SPE-J are most closely related to SMEZ and SPEC, whereas SPEH is most similar to the SEs than to any other streptococcal toxin.

As described by Proft, T et al supra, rSMEZ, rSMEZ-2, rSPE-G, and rSPE-H were mitogenic for human peripheral blood T lymphocytes. SMEZ-2 appears to be the most potent SAg discovered thus far.

All these toxins, except rSPE-G, were active on murine T cells, but with reduced potency.

Binding to a human B-lymphoblastoid line was shown to be zinc dependent with high binding affinity of 15-65 nM. Analysis of competition for binding between toxins of this group revealed overlapping but discrete binding to subsets of class II molecules in the hierarchical order (SMEZ, SPE-C)->SMEZ-2>SPE-H>SPE-G. The most common targets for these SAgs were human Vβ2.1- and Vβ4-expressing T cells.

Streptococcus Pyrogenic Exotoxin A (SPEA)

SPEA can be purified from cultures of S. pyogenes as described by Kline et al., Infect. Immun. 64:861-869 (1996). Plasmids that include the spea1 gene which encode SPEA, and the expression and purification of recombinant SPEA (“rSPEA”) are described by Kline et al., supra. The native SPEA sequence is shown below:

| SPEA (Papageorgiou, A. C. et al, EMBO J. 18: 9-21 (1999)) | |
|---|---|
| [SEQ ID NO: 25] | |
| 1 | MENNKKVLKK MVFFVLVTFL GLTISQEVFA QQDPDPSQLH RSSLVKNLQN |
| | IYFLYEGDPV |
| 61 | THENVKSVDQ LLSHDLIYNV SGPNYDKLKT ELKNQEMATL FKDKNVDIYG |
| | VEYYHLCYLC |
| 121 | ENAERSACIY GGVTNHEGNH LEIPKKIVVK VSIDGIQSLS FDIETNKKMV |
| | TAQELDYKVR |
| 181 | KYLTDNKQLY TNGPSKYETG YIKFIPKNKE SFWFDFFPEP EFTQSKYLMI |
| | YKDNETLDSN |
| 241 | TSQIEVYLTT K |

Streptococcus Pyrogenic Exotoxin B (SPEB)

Purification of native SPEB is described by Gubba, S. et al., Infect. Immun. 66: 765-770 (1998). Expression and purification of recombinant SPEB are also described in this reference. The native SPEB sequence is shown below (Kapur, V. et al., Microb. Pathog. 15:327-346 (1993)):

| [SEQ ID NO: 26] | |
|---|---|
| 1 | MNKKKLGIRL LSLLALGGFV LANPVFADQN FARNEKEAKD SAITFIQKSA |
| | AIKAGARSAE |
| 61 | DIKLDKVNLG GELSGSNMYV YNISTGGFVI VSGDKRSPEI LGYSTSGSFD |
| | ANGKENIASF |
| 121 | MESYVEQIKE NKKLDTTYAG TAEIKQPVVK SLLDSKGIHY NQGNPYNLLT |
| | PVIEKVKPGE |
| 181 | QSFVGQHAAT GCVATATAQI MKYHNYPNKG LKDYTYTLSS NNPYFNHPKN |
| | LFAAISTRQY |
| 241 | NWNNILPTYS GRESNVQKMA ISELMADVGI SVDMDYGPSS GSAGSSRVQR |
| | ALKENFGYNQ |
| 301 | SVHQINRSDF SKQDWEAQID KELSQNQPVY YQGVGKVGGH AFVIDGADGR |
| | NFYHVNWGWG |
| 361 | GVSDGFFRLD ALNPSALGTG GGAGGFNGYQ SAVVGIKP |

Streptococcus Pyrogenic Exotoxin C(SPEC)

Methods of isolation and characterization of SPEC is carried out by the methods of L1, P L et al., J. Exp. Med. 186: 375-383 (1997). These references also describe T cell proliferation stimulated by this SAg and the analysis of its selectivity for TCR Vβ regions. The native sequence of SPEC (Kapur, V. et al., Infect. Immun. 60: 3513-3517 (1992)) is shown below:

[SEQ ID NO: 27]

| | |
|---|---|
| 1 | MKKINIIKIV FIITVILIST ISPIIKSDSK KDISNVKSDL |
| | LYAYTITPYD YKDCRVNFST |
| 61 | THTLNIDTQK YRGKDYYISS EMSYEASQKF |
| | KRDDHVDVFG LFYILNSHTG EYIYGGITPA |
| 121 | QNNKVNHKLL GNLFISGESQ QNLNNKIILE |
| | KDIVTFQEID FKIRKYLMDN YKIYDATSPY |
| 181 | VSGRIEIGTK DGKHEQIDLF DSPNEGTRSD |
| | IFAKYKDNRI INMKNFSHFD IYLE |

Streptococcal Superantigen (SSA)

SSA is a ˜28-kDa superantigen protein isolated from culture supernatants as described by Mollick J et al., J. Clin. Invest. 92: 710-719 (1993) and Reda K et al., Infect. Immun. 62: 1867-1874 (1994). SSA stimulates proliferation of human T cells bearing Vβ1, Vβ3, Vβ5.2, and Vβ15 in an MHC class II-dependent manner. The first 24 amino acid residues of SSA are 62.5% identical to SEB, SEC1, and SEC3. Purification and cloning of SSA is described in Reda K et al., Infect. Immun. 62: 1867-1874 (1994). The native sequence of SSA (Reda, K B. et al., Infect. Immun. 64: 1161-1165 (1996)) is shown below:

[SEQ ID NO: 28]

| | |
|---|---|
| 1 | MNKRIRILVV ACVVFCAQLL SISVFASSQP DPTPEQLNKS |
| | SQFTGVMGNL RCLYDNHFVE |
| 61 | GTNVRSTGQL LQHDLIFPIK DLKLKNYDSV KTEFNSKDLA |
| | AKYKNKDVDI FGSNYYYNCY |
| 121 | YSEGNSCKNA KKTCMYGGVT EHHRNQIEGK FPNITVKVYE |
| | DNENILSFDI TTNKKQVTVQ |
| 181 | ELDCKTRKIL VSRKNLYEFN NSPYETGYIK FIESSGDSFW |
| | YDMMPAPGAI FDQSKYLMLY |
| 241 | NDNKTVSSSA IAIEVHLTKK |

Streptococcal Pyrogenic Exotoxins G and H and SMEZ

The sequences of the more recently discovered Streptococcal exotoxin SAgs are provided below:

| SPEG (Fraser, J et al., Mol Med Today 6: 125-32 (2000)) | |
|---|---|
| [SEQ ID NO: 29] | |
| 1 | DENLKDLKRS LRFAYNITPC DYENVEIAFV TTNSIHINTK QKRSECILYV |
| | DSIVSLGITD |
| 61 | QFIKGDKVDV FGLPYNFSPP YVDNIYGGIV KHSNQGNKSL QFVGILNQDG |
| | KETYLPSEVV |
| 121 | RIKKKQFTLQ EFDFKIRKFL MEKYNIYDSE SRYTSGSLFL ATKDSKHYEV |
| | DLFNKDDKLL |
| 181 | SRDSFFKRYK DNKIFNSEEI SHFDIYLKTY |
| SPEH (Proft, T. et al., J. Exp. Med. 189: | |
| 89-102 (1999)) | |
| [SEQ ID NO: 30] | |
| 1 | MRYNCRYSHI DKKIYSMIIC LSFLLYSNVV QANSYNTTNR HNLESLYKHD |
| | SNLIEADSIK |
| 61 | NSPDIVTSHM LKYSVKDKNL SVFFEKDWIS QEFKDKEVDI YALSAQEVCE |
| | CPGKRYEAFG |
| 121 | GITLTNSEKK EIKVPVNVWD KSKQQPPMFI TVNKPKVTAQ EVDIKVRKLL |
| | IKKYDIYNNR |
| 181 | EQKYSKGTVT LDLNSGKDIV FDLYYFGNGD FNSMLKIYSN NERIDSTQFH |
| | VDVSIS |
| SMEZ (Proft, T. et al., J. Exp. Med. 191: | |
| 1765-1776 (2000)) | |
| [SEQ ID NO: 31] | |
| 1 | LEVDNNSLLR NIYSTIVYEY SDTVIDFKTS HNLVTKKLDV RDARDFFINS |
| | EMDEYAANDF |
| 61 | KAGDKIAVFS VPFDWNYLSK GKVTAYTYGG ITPYQKTSIP KNIPVNLWIN |
| | RKQIPVPYNQ |
| 121 | ISTNKTTVTA QEIDLKVRKF LIAQHQLYSS GSSYKSGKLV FHTNDNSDKY |
| | SLDLFYTGYR |
| 181 | DKESIFKVYK DNKSFNIDKI GHLDIEIDS |
| SMEZ 2 (Arcus, V. L. et al., J. Mol. Biol. 299 (1), | |
| 157-168 (2000)) | |
| [SEQ ID NO: 32] | |
| 1 | GLEVDNNSLL RNIYSTIVYE YSDIVIDFKT SHNLVTKKLD VRDARDFFIN |
| | SEMDEYAAND |
| 61 | FKTGDKIAVF SVPFDWNYLS KGKVTAYTYG GITPYQKTSI PKNIPVNLWI |
| | NGKQISVPYN |
| 121 | EISTNKTTVT AQEIDLKVRK FLIAQHQLS SGSSYKSGRL VFHTNDNSDK |
| | YSFDLFYVGY |
| 181 | RDKESIFKNY KDNKSFNIDK IGHLDIEIDS |

Yersinia pseudotuberculosis Mitogen (Superantigen) (YPM)

Cloning, expression and purification of YPM is described by Miyoshi-Akiyama, T. et al., J. Immunol. 154: 5228-5234 (1995). The above reference described assays of YPM using lymphoid cells and murine L cells transfected with human HLA genes, including T cell proliferation and cytokine (IL2) secretion. The sequence of YPM is shown below

(Carnoy, C. et al., J. Bacteriol. 184 (16),
4489-4499 (2002))

[SEQ ID NO: 33]:

| | |
|---|---|
| 1 | MKKKFLSLLT LTFFSGLALA ADYDNTLNSI PSLRIPNIET |
| | YTGTIQGKGE VCIRGNKEGK |
| 61 | SRGGELYAVL RSTNANADMT LILLCSIRDG WKEVKRSDID |
| | RPLRYEDYYT PGALSWIWEI |
| 121 | KNNSSEASDY SLSATVHDDK EDSDVLMKCP |

Staphylococcal Exotoxin Like Proteins (SET)

The identification characterization of the SETs (SET-1 and SET-2) and the cloning and purification of SET-1 is described in Williams, R. J. et al., Infect. Immun. 68: 4407-4414 (2000). This reference discloses the distribution of the sell gene among Staphylococcal species and strains. The set1 nucleotide sequences are deposited in the GenBank database under accession numbers AF094826 (set gene cluster fragment), AF188835 (NCTC 6571 set1 gene), AF 188836 (FR1326 set1 gene), and AF 188837 (NCTC 8325-4 set1 gene). Recombinant SET-1 protein stimulates production of the proinflammatory cytokines IL-1β, IL-6, and TNFα

| SET1 (Williams, R. J. et al., Infect. Immun. 68 (8), | |
|---|---|
| 4407-4415 (2000)) | |
| [SEQ ID NO: 34] | |
| 1 | MKLKTLAKAT LALSLLTTGV ITLESQAVKA AEKQERVQHL YDIKDLYRYY |
| | SAPSFEYSNI |
| 61 | SGKVENYNGS NVVRFNQKDQ NHQLFLLGKD KEQYKEGLQG KDVFVVQELI |
| | DPNGRLSTVG |
| 121 | GVTKKNNKTS ETKTHLLVNK VDGGNLDASI DSFLIQKEEI SLKELDFKIR |
| | QQLVEKYGLY |
| 181 | QGTSKYGKIT INLKDEKREV IDLSDKLEFE RMGDVLNSKD IKGISVTINQ I |
| SET2 Williams, R. J., et al., Infect. Immun. 68 (8), | |
| 4407-4415 (2000) | |
| [SEQ ID NO: 35] | |
| 1 | MKLKTLAKAT LALGLLTTGV ITSEGQAVQA AEKQERVQHL HDIRDLHRYY |
| | SSESFEYSNV |
| 61 | SGKVENYNGS NVVRFNPKDQ NHQLFLLGKD KEQYKEGLQG QNVFVVQELI |
| | DPNGRLSTVG |
| 121 | GVTKKNNKTS ETNTPLFVNK VNGEDLDASI DSFLIQKEEI SLKELDFKIR |
| | QQLVNNYGLY |
| 181 | KGTSKYGKII INLKDENKVE IDLGDKLQFE RMGDVLNSKD IRGISVTINQ I |
| SET3 (Williams, R. J. et al., Infect. Immun. 68 (8), | |
| 4407-4415 (2000)) | |
| [SEQ ID NO: 36] | |
| 1 | MKMTAIAKAS LALSILATGV ITSTAQTVNA SEHESKYENV TJDUFDKRDT |
| | YSRASKELKN |
| 61 | VTGYRSKGG KKHYLIFDKNR KFTRIQIFGK DIERIKKRKN PGLDIFVVKE |
| | AENRNGTVYS |
| 121 | YGGVTLLMQG AYYDYLSAPR FVIKKEVGAG VSVHVKRYYI YKEEISLKEL |
| | DFKLRQYLIQ |
| 181 | DFDLYKKFPK ASKIKVTMKD GGYYTFELNK KLQTNRMSDV IDGRNIEKIE ANIR |
| SET4 (Williams, R. J. et al., Infect. Immun. 68 (8), | |
| 4407-4415 (2000)) | |
| [SEQ ID NO: 37] | |
| 1 | MKLTALAKVT LALGILTTGT LTTEAHSGHA KQNQKSVNKH DKEALHRYYT |
| | GNFKEMKNIN |
| 61 | ALRHGKNNLR FKYRGMKTQV LLPBDEYRKY QQRRHTGLDV FFNQERRDKH |
| | DISYTVGGVT |
| 121 | KTNKTSGFVS TPRLNVTKEK GEDAFVKGYP YDIKKEEISL KELDFKLRKH |
| | LIEKYGLYKT |
| 181 | LSKDGRIKIS LKDGSFYNLD LRTKLKFKHM GEVIDSKQIK DIEVNLK |
| SET5 (Williams, R. J., et al., Infect. Immun. 68 (8), | |
| 4407-4415 (2000)) | |
| [SEQ ID NO: 38] | |
| 1 | MKLTAIAKAT LALGILTTGV MTAESQTVNA KVKLDETQRK YYINMLKDYY |
| | SQESYESTNI |
| 61 | SVKSEDYYGS NVLNFNQRNK NFKVFLIGDD RNKYKELTHG RDVFAVPELI |
| | DTKGGIYSVG |
| 121 | GITKKNVRSV FGYVSHPGLQ VKKVDPKDGF SIKELFFIQK EEVSLKELDF |
| | KIRKMLVEKY |
| 181 | RLYKGASDKG RIVINMKDEK KHEIDLSEKL SFDRMFDVLD SKQIKNIEVN LN |

### Functional Homologues and Derivatives of Tumoricidal Proteins, Superantigens or Peptides

The present invention contemplates, in addition to native proteins incorporated in the sickle cells, the use of homologues of native proteins that have the requisite biological activity to be useful in accordance with the invention.

Thus, in addition to native proteins and nucleic acid compositions described herein, the present invention encompasses functional derivatives, among which homologues are preferred. By “functional derivative” is meant a “fragment,” “variant,” “mutant,” “homologue,” “analogue,” or “chemical derivative. Homologues include fusion proteins, chimeric proteins and conjugates that include a SAg portion fused to or conjugated to a fusion partner polypeptide or peptide. A functional derivative retains at least a portion of the biological activity of the native protein which permits its utility in accordance with the present invention. For superantigens, such biological activity includes stimulation of T cell proliferation and/or cytokine secretion, stimulation of T cell-mediated cytotoxic activity, as a result of interactions of the SAg composition with T cells preferably via the TCR Vβ or Vα region. For pseudomonas exotoxin A, a homologue must retain tumor cell cytolytic activity.

A “fragment” refers to any shorter peptide. A “variant” refers to a molecule substantially similar to either the entire protein or a peptide fragment thereof. Variant peptides may be conveniently prepared by direct chemical synthesis of the variant peptide, using methods well-known in the art.

A homologue refers to a natural protein, encoded by a DNA molecule from the same or a different species. Homologues, as used herein, typically share at least about 50% sequence similarity at the DNA level or at least about 18% sequence similarity at the amino acid level, with a native protein.

An “analogue” refers to a non-natural molecule substantially similar to either the entire molecule or a fragment thereof.

A “chemical derivative” contains additional chemical moieties not normally a part of the peptide. Covalent modifications of the peptide are included within the scope of this invention. Such modifications may be introduced into the molecule by reacting targeted amino acid residues of the peptide with an organic derivatizing agent that is capable of reacting with selected side chains or terminal residues.

A fusion protein comprises a native protein, a fragment or a homologue fused by recombinant means to another polypeptide fusion partner, optionally including a spacer between the two sequences. Preferred fusion partners are antibodies, Fab fragments, single chain Fv fragments. Other fusion partners are any peptidic receptor, ligand, cytokine, domain (“ECD”) of a molecule and the like.

The recognition that the biologically active regions of the proteins, for example, are substantially homologous, i.e., that the sequences are substantially similar, enables prediction of the sequences of synthetic peptides which will exhibit similar biological effects in accordance with this invention.

The following terms are used in the disclosure of sequences and sequence relationships between two or more nucleic acids or polypeptides: (a) “reference sequence”, (b) “comparison window”, (c) “sequence identity”, (d) “percentage of sequence identity”, and (e) “substantial identity”

As used herein, “reference sequence” is a defined sequence used as a basis for sequence comparison. A reference sequence may be a subset or the entirety of a specified sequence; for example, as a segment of a full-length cDNA or other polynucleotide sequence, or the complete cDNA or polynucleotide sequence. The same is the case for polypeptides and their amino acid sequences.

As used herein, “comparison window” includes reference to a contiguous and specified segment of a polynucleotide or amino acid sequence, wherein the sequence may be compared to a reference sequence and wherein the portion of the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides or amino acids in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of nucleotide and amino acid sequences for comparison are well-known in the art. For comparison, optimal alignment of sequences may be done using any suitable algorithm, of which the following are examples:
(a) the local homology algorithm (“Best Fit”) of Smith and Waterman, Adv. Appl. Math. 2: 482 (1981);
(b) the homology alignment algorithm (GAP) of Needleman and Wunsch, J. Mol. Biol. 48: 443 (1970); or
(c) a search for similarity method (FASTA and TFASTA) of Pearson and Lipman, Proc. Natl. Acad. Sci. 85 2444 (1988);

In a preferred method of alignment, Cys residues are aligned. Computerized implementations of these algorithms, include, but are not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG) (Madison, Wis.). The CLUSTAL program is described by Higgins et al., Gene 73:237-244 (1988); Higgins et al., CABIOS 5:151-153 (1989); . Corpet et al., Nuc Acids Res 16:881-90 (1988); Huang et al., CABIOS 8:155-65 (1992), and Pearson et al., Methods in Molecular Biology 24:307-331 (1994).

A preferred program for optimal global alignment of multiple sequences is PileUp (Feng and Doolittle, J Mol Evol 25:351-360 (1987) which is similar to the method described by Higgins et al., 1989, supra).

The BLAST family of programs which can be used for database similarity searches includes: NBLAST for nucleotide query sequences against database nucleotide sequences; XBLAST for nucleotide query sequences against database protein sequences; BLASTP for protein query sequences against database protein sequences; TBLASTN for protein query sequences against database nucleotide sequences; and TBLASTX for nucleotide query sequences against database nucleotide sequences. See, for example, Ausubel et al., eds., Current Protocols in Molecular Biology, Chapter 19, Greene Publishing and Wiley-Interscience, New York (1995) or most recent edition. Unless otherwise stated, stated sequence identity/similarity values provided herein, typically in percentages, are derived using the BLAST 2.0 suite of programs (or updates thereof) using default parameters. Altschul et al., Nuc Acids Res. 25:3389-3402 (1997).

As is known in the art, BLAST searches assume that proteins can be modeled as random sequences. However, many real proteins comprise regions of nonrandom sequence which may include homopolymeric tracts, short-period repeats, or regions rich in particular amino acids. Alignment of such regions of “low-complexity” regions between unrelated proteins may be performed even though other regions are entirely dissimilar. A number of low-complexity filter programs are known that reduce such low-complexity alignments. For example, the SEG (Wooten et al., Comput. Chem. 17:149-163 (1993) and XNU (Claverie et al., Comput. Chem., 17:191-201 (1993) low-complexity filters can be employed alone or in combination.

As used herein, “sequence identity” or “identity” in the context of two nucleic acid or amino acid sequences refers to the residues in the two sequences which are the same when aligned for maximum correspondence over a specified comparison window. It is recognized that when using percentages of sequence identity for proteins, a residue position which is not identical often differs by a conservative amino acid substitution, where a substituting residue has similar chemical properties (e.g., charge, hydrophobicity, etc.) and therefore does not change the functional properties of the polypeptide. Where sequences differ in conservative substitutions, the % sequence identity may be adjusted upwards to correct for the conservative nature of the substitution, and be expressed as “sequence similarity” or “similarity” (combination of identity and differences that are conservative substitutions). Means for making this adjustment are well-known in the art. Typically this involves scoring a conservative substitution as a partial rather than as a full mismatch, thereby increasing the percentage sequence identity. Thus, for example, where an identical amino acid is given a score of “1” and a non-conservative substitution is given a score of “0” zero, a conservative substitution is given a score between 0 and 1. The scoring of conservative substitutions is calculated, e.g., according to the algorithm of Meyers et al., CABIOS 4:11-17 (1988) as implemented in the program PC/GENE (Intelligenetics, Mountain View, Calif., USA).

As used herein, “percentage of sequence identity” refers to a value determined by comparing two optimally aligned sequences over a comparison window, wherein the portion of the nucleotide or amino acid sequence in the comparison window may comprise additions or deletions (i.e., gaps) as compared to the reference sequence (which lacks such additions or deletions) for optimal alignment, such as by the GAP algorithm (supra). The percentage is calculated by determining the number of positions at which the identical nucleotide or amino acid residue occurs in both sequences to yield the number of matched positions, dividing that number by the total number of positions in the window of comparison and multiplying the result by 100, thereby calculating the percentage of sequence identity.

The term “substantial identity” of two sequences means that a polynucleotide or polypeptide comprises a sequence that has at least 60%, preferably at least 70%, more preferably at least 80%, even more preferably at least 90%, and most preferably at least 95% sequence identity to a reference sequence using one of the alignment programs described herein using standard parameters. Values can be appropriately adjusted to determine corresponding identity of the proteins encoded by two nucleotide sequences by taking into account codon degeneracy, amino acid similarity, reading frame positioning, etc.

One indication that two nucleotide sequences are substantially identical is if they hybridize to one other under stringent conditions. Because of the degeneracy of the genetic code, a number of different nucleotide codons may encode the same amino acid. Hence, two given DNA sequences could encode the same polypeptide but not hybridize under stringent conditions. Another indication that two nucleic acid sequences are substantially identical is that the polypeptide encoded by the first nucleic acid is immunologically cross reactive with the polypeptide encoded by the second nucleic acid. Clearly, then, two peptide or polypeptide sequences are substantially identical if one is immunologically reactive with antibodies raised against the other. A first peptide is substantially identical to a second peptide, if they differ only by a conservative substitution. Peptides which are “substantially similar” share sequences as noted above except that nonidentical residue positions may differ by conservative substitutions.

Thus, in one embodiment of the present invention, the Lipman-Pearson FASTA or FASTP program packages (Pearson, W. R. et. al., 1988, supra; Lipman, D. J. et al, Science 227:1435-1441 (1985)) in any of its older or newer iterations may be used to determine sequence identity or homology of a given protein, preferably using the BLOSUM 50 or PAM 250 scoring matrix, gap penalties of −12 and −2 and the PIR or SwissPROT databases for comparison and analysis purposes. The results are expressed as z values or E( ) values. To achieve a more “updated” z value cutoff for statistical significance, preferably corresponding to a z value >10 based on the increase in database size over that of 1988, in a FASTA analysis using the equivalent 2001 database, a significant z value would exceed 13.

A more widely used and preferred methodology determines the percent identity of two amino acid sequences or of two nucleic acid sequences after optimal alignment as discussed above, e.g., using BLAST. In a preferred embodiment of this approach, a polypeptide being analyzed for its homology with native protein is at least 20%, preferably at least 40%, more preferably at least 50%, even more preferably at least 60%, and even more preferably at least 70%, 80%, or 90% as long as the reference sequence. The amino acid residues (or nucleotides) at corresponding positions are then compared. Amino acid or nucleic acid “identity” is equivalent to amino acid or nucleic acid “homology”.

In a preferred comparison of a putative polypeptide or peptide homologue polypeptide and a native protein, the percent identity between two amino acid sequences is determined using the Needleman and Wunsch alignment algorithm (incorporated into the GAP program in the GCG software package (available at the URL www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between the encoding nucleotide sequences is determined using the GAP program in the GCG software package (also available at above URL), using a NWSgapdna.CMP matrix and a gap weight of 40, 50, 60, 70, or 80 and a length weight of 1, 2, 3, 4, 5, or 6. In another embodiment, the algorithm of Meyers et al., supra (incorporated into the ALIGN program, version 2.0), is implemented using a PAM 120 weight residue table, a gap length penalty of 12 and a gap penalty of 4.

The wild-type (or native) SAg-encoding nucleic acid sequence or the SAg protein sequence can further be used as a “query sequence” to search against a public database, for example, to identify other family members or related sequences. Such searches can be performed using the NBLAST and XBLAST programs, supra (see Altschul et al. (1990) J. Mol. Biol. 215:403-10). BLAST nucleotide searches can be performed with the NBLAST program, score=100, wordlength=12 to identify nucleotide sequences homologous to native SAgs. BLAST protein searches can be performed with the XBLAST program, score=50, wordlength=3 to identify amino acid sequences homologous to identify polypeptide molecules homologous to a native SAg. To obtain gapped alignments for comparison purposes, Gapped BLAST can be utilized as described in Altschul et al. (1997, supra). Default parameters of XBLAST and NBLAST can be found at the NCBI website (www.ncbi.nlm.nih.gov)

Using the FASTA programs and method of Pearson and Lipman, a preferred SAg homologue is one that has a z value >10. Expressed in terms of sequence identity or similarity, a preferred SAg homologue for use according the present invention has at least about 20% identity or 25% similarity to native SAg. Preferred identity or similarity is higher. More preferably, the amino acid sequence of a homologue is substantially identical or substantially similar to a native protein molecule as those terms are defined above.

One group of substitution variants (also homologues) are those in which at least one amino acid residue in the peptide molecule, and preferably, only one, has been removed and a different residue inserted in its place. Deletion and addition variants are also homologues if they satisfy the structural and functional criteria set forth herein with respect to their parent or native molecules. For a detailed description of protein chemistry and structure, see Schulz, G. E. Principles of Protein Structure Springer-Verlag, New York, 1978, and Creighton, T. E., Proteins: Structure and Molecular Properties, W.H. Freeman & Co., San Francisco, 1983, which are hereby incorporated by reference. The types of substitutions which may be made in the protein or peptide molecule of the present invention may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:

1. Small aliphatic, nonpolar or slightly polar residues: Ala, Ser, Thr (Pro, Gly);
2. Polar, negatively charged residues and their amides: Asp, Asn, Glu, Gln;
Polar, positively charged residues: H is, kg, Lys;
4. Large aliphatic, nonpolar residues: Met, Leu, Ile, Val (Cys); and
5. Large aromatic residues: Phe, Tyr, Trp.

The three amino acid residues in parentheses above have special roles in protein architecture. Gly is the only residue lacking any side chain and thus imparts flexibility to the chain. Pro, because of its unusual geometry, tightly constrains the chain. Cys can participate in disulfide bond formation which is important in protein folding. Tyr, because of its hydrogen bonding potential, has some kinship with Ser, Thr, etc.

More substantial changes in functional or immunological properties are made by selecting substitutions that are less conservative, such as between, rather than within, the above five groups, which will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (a) substitution of gly and/or pro by another amino acid or deletion or insertion of Gly or Pro; (b) substitution of a hydrophilic residue, e.g., Ser or Thr, for (or by) a hydrophobic residue, e.g., Leu, Ile, Phe, Val or Ala; (c) substitution of a Cys residue for (or by) any other residue; (d) substitution of a residue having an electropositive side chain, e.g., Lys, Arg or H is, for (or by) a residue having an electronegative charge, e.g., Glu or Asp; or (e) substitution of a residue having a bulky side chain, e.g., Phe, for (or by) a residue not having such a side chain, e.g., Gly.

The deletions and insertions, and substitutions according to the present invention are those which do not produce radical changes in the characteristics of the protein or peptide molecule. However, when it is difficult to predict the exact effect of the substitution, deletion, or insertion in advance of doing so, one skilled in the art will appreciate that the effect will be evaluated by routine screening assays, for example direct or competitive immunoassay of cytotoxicity or biological assay of T cell function as described herein. For non-superantigen homologues, the screening test(s) selected to assay function reflect the intrinsic functional activity of the native protein particularly its tumoricidal activity in the context of the inventions described herein. Modifications of such proteins or peptide properties as redox or thermal stability, hydrophobicity, susceptibility to proteolytic degradation or the tendency to aggregate with carriers or into multimers are assessed by methods well known to the ordinarily skilled artisan.

### Chemical Derivatives

Covalent modifications of the SAg proteins or peptide fragments thereof, preferably of SEs or peptide fragments thereof, are included herein. Such modifications may be introduced into the molecule by reacting targeted amino acid residues of the protein or peptide with an organic derivatizing agent that is capable of reacting with selected side chains or terminal residues. This may be accomplished before or after polymerization.

Cysteinyl residues most commonly are reacted with a-haloacetates (and corresponding amines), such as 2-chloroacetic acid or chloroacetamide, to give carboxymethyl or carboxyamidomethyl derivatives. Cysteinyl residues also are derivatized by reaction with bromotrifluoroacetone, α-bromo-(5-imidozoyl) propionic acid, chloroacetyl phosphate, N-alkylmaleimides, 3-nitro-2-pyridyldisulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing a-amino-containing residues include imidoesters such as methyl picolinimidate; pyridoxal phosphate; pyridoxal; chloroborohydride; trinitrobenzenesulfonic acid; 0-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pK of the guanidine functional group. Furthermore, these reagents may react with the groups of lysine as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction with carbodiimides as noted above. Aspartyl and glutamyl residues are converted to asparaginyl and glutaminyl residues by reaction with ammonium ions.

Glutaminyl and asparaginyl residues may be deamidated to the corresponding glutamyl and aspartyl residues. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the a-amino groups of lysine, arginine, and histidine side chains (T. E. Creighton, Proteins: Structure and Molecule Properties, W.H. Freeman & Co., San Francisco, pp. 79-86 (1983)), acetylation of the N-terminal amine, and, in some instances, amidation of the C-terminal carboxyl groups.

Such derivatized moieties may improve the solubility, absorption, biological half life, and the like. The moieties may alternatively eliminate or attenuate any undesirable side effect of the protein and the like. Moieties capable of mediating such effects are disclosed, for example, in Remington's Pharmaceutical Sciences, 16th ed., Mack Publishing Co., Easton, Pa. (1980).

### Superantigen Homologues

The variants or homologues of native SAg proteins or peptides including mutants (substitution, deletion and addition types), fusion proteins (or conjugates) with other polypeptides, are characterized by substantial sequence homology to
(a) the long-known SE's—SEA, SEB, SEC1-3, SED, SEE and TSST-1;
(b) long-known SpE's;
(c) more recently discovered SE's (SEG; SEH, SEI, SEJ, SEK, SEL, SEM, SEN, SEO, SEP, SER, SEU, SETs 1-5); or
(d) non-enterotoxin superantigens (YPM, M. arthritides superantigen).
   Preferred homologues were disclosed above.

Table 1 in PCT US05/022638 filed Jun. 27, 2005 incorporated in its entirety by reference lists a number of native SEs and exemplary homologues (amino acid substitution, deletion and addition variants (mutants) and fragments) with z values >10 (range: z=16 to z=136) using the Lipman-Pearson algorithm and FASTA. These homologues also induce significant T lymphocyte mitogenic responses that are generally comparable to native SE's.

In addition, as shown in Table 2 of PCT US05/022638 filed Jun. 27, 2005 incorporated in its entirety by reference, several of these homologues also promote antigen-nonspecific T lymphocyte killing in vitro by a mechanism termed “superantigen-dependent cellular cytotoxicity” (SDCC) or, in the case of SAg-mAb fusion proteins, “superantigen/antibody dependent cellular cytotoxicity (SADCC).” According to the present invention, other SE homologues (e.g., z>10 or, in another embodiment, having at least about 20% sequence identity or at least about 25% sequence similarity when compared to native SEs), exhibiting T lymphocyte mitogenicity, SDCC or SADCC, are useful anti-tumor agents when administered to a tumor bearing host.

### Pharmaceutical Compositions and Administration

The sickled erythrocytes may be administered parenterally preferably intravenously by infusion or injection but also may be implanted or injected intratumorally, intrapleurally, intrathecally, intrapericardially, intravesicularly, subcutaneously, intralymphatically, intraarticularly, intradermally, intracranially, intraarticularly or intramuscularly. They may be administered in a controlled release formulation.

The pharmaceutical compositions of the present invention will generally comprise an effective amount of sickled erythrocytes dissolved or dispersed in a pharmaceutically acceptable carrier or aqueous medium. One or more administrations may be employed, depending upon the lifetime of the drug at the tumor site and the response of the tumor to the drug. Administration may be by syringe, catheter or other convenient means allowing for introduction of a flowable composition. Administration may be every three days, weekly, or less frequent, such as biweekly or at monthly intervals.

The phrases “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by U.S. Food and Drug Administration. Supplementary active ingredients can also be incorporated into the compositions.

“Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

### Injectable Formulations

The sickle cells compositions of the present invention are preferably formulated for parenteral administration, e.g., introduction by injection, infusion. They may also be administered intravenously, intramuscularly, intradermally, intraperitoneally, intrapleurally, intraarticularly. Means for preparing aqueous compositions that contain the SAg compositions are known to those of skill in the art in light of the present disclosure. Typically, such compositions can be prepared as for a typical blood transfusion, either as liquid solutions or suspensions; solid forms suitable for using to prepare solutions or suspensions upon the addition of a liquid prior to injection can also be prepared.

The techniques of preparation are generally well known in the art as exemplified by Remington's Pharmaceutical Sciences, 16th Ed. Mack Publishing Company, 1980, or most recent edition, incorporated herein by reference. Moreover, for human administration, preparations should meet sterility, pyrogenicity, general safety and purity standards as required by the U.S. Food and Drug Administration. Upon formulation, the therapeutic compositions are administered in a manner compatible with the dosage formulation and in such amount as is therapeutically effective.

### Animal and Human Testing

The effects of murine sickled erythrocytes are tested murine tumor models as given in the section titled “Tumor Models and Procedures for Evaluating Anti-Tumor Effects Studies” (pp. 72-82 instant specification). The sickled erythrocytes are obtained from mice with models of sickle cell disease as shown in the Table 5 below of mouse models and also include from mice with sickle-thalassemia containing little or no HbA hemoglobin.

TABLE 5
Transgenic Murine models for Sickle Cell disease

| Mouse | Mouse_{—} | Mouse_{—} | Human_{—} | Human_ [symbol] | |
|---|---|---|---|---|---|
| Model | Globin | Globin | Globin | Globin | %_ [symbol] |
| Normal | Yes | Yes | No | No | 0 |
| New York | Yes | Deleted | No | _ [symbol] | 32 |
| (NY) | | | | | |
| Berkeley | Yes | Deleted | No | _ [symbol] ^{-Antilles} | 29 |
| (B) | | | | | |
| Hybrid(H) | Yes | Deleted | No | _ [symbol] | 42.2 |
| [NY_B] | | | | _ [symbol] ^{-Antilles} | 35.9 |
| Paszty(P) | Deleted | Deleted | Yes | _ [symbol] | 39 |

[symbol]

indicates data missing or illegible when filed

For testing any of the above transgenic mouse models of sickle cell disease or trait in the C57/Bl background are useful. Other murine genetic backgrounds are suitable as well. The tumors are those indigenous to the mouse strain of the donor sickled erythrocytes. In one example, it would be the MCA 205-207 sarcoma which is indigenous to the C57/Bl mouse. Other tumors indigenous to this strain are the B16F10 melanoma, Lewis lung carcinoma, CT-26 colon carcinoma and hepatocellular carcinomas. In a typical experiment, C57Bl mice with established Lewis Lung carcinomas are injected intravenously with 0.1-0.2 ml of nucleated erythrocytes from a homozygous SS mouse. The SS erythrocytes may also be transduced with nucleic acids encoding a superantigen or toxins given above, a hemolysin, oncolytic virus or anaerobic bacterial spore fused to a CMV promoter optionally with an HRE element The injected SS cells aggregate and cluster in the tumor vasculature using real time intravital microscopy. Repeated injections every 2-7 days produce an objective reduction of tumor mass.

Human studies are described in Example 3.

### Tumor Models and Procedures for Evaluating Anti-Tumor Effects Studies

The various sickle cell compositions described herein are tested for therapeutic efficacy in several well established rodent models which are considered to be highly representative of a broad spectrum of human tumors. These approaches are described in detail in Geran, R. I. et al., “Protocols for Screening Chemical Agents and Natural Products against Animal Tumors and Other Biological Systems (Third Edition)”, Canc. Chemother. Reports, Pt 3, 3:1-112, which is hereby incorporated by reference in its entirety.

In general the SS cells, SA cells, SS variant cells, SS progenitors, erythroleukemia cells, erythroleukemia cells transfected with BCAM/LU, SS porphyric cells, SS ghosts are loaded with tumoricidal virus, protein, drug, toxin, antibody, toxin-antibody conjugate as and optionally pre-treated with light therapy or photosensitizers as described as described herein. The cells are administered to tumor bearing mice by intravenous infusion or injection in doses of 0.05 to 0.20 ml over 30 seconds to 2 minutes. The treatment is repeated every day or every second or third day for up to 10 treatments.

### A. Calculation of Mean Survival Time (MST)

MST (days) is calculated according to the formula:

$$\frac{S + AS ( A - 1 ) - ( B + 1 ) NT}{S ( A - 1 ) - NT}$$

Day: Day on which deaths are no longer considered due to drug toxicity. For example, with treatment starting on Day 1 for survival systems (such as L1210, P388, B16, 3LL, and W256): Day A=Day 6; Day B=Day beyond which control group survivors are considered “no-takes.”
S: If there are “no-takes” in the treated group, S is the sum from Day A through Day B. If there are no “no-takes” in the treated group, S is the sum of daily survivors from Day A onward.
S(A-1): Number of survivors at the end of Day (A-1).
Example: for 3LE21, S(A-1)=number of survivors on Day 5.
NT: Number of “no-takes” according to the criteria given in Protocols 7.300 and 11.103.

### B. T/C Computed for all Treated Groups

$$T / C = \frac{MST of treated group}{MST of control group} \times 100$$

Treated group animals surviving beyond Day Bare eliminated from calculations (as follows):

| No. of survivors in treated | Percent of “no-takes” | |
|---|---|---|
| group beyond Day B | in control group | Conclusion |
| 1 | Any percent | “no-take” |
| 2 | <10 | drug inhibition |
| | ³10 | “no-takes” |
| ³3 | <15 | drug inhibitions |
| | ³15 | “no-takes” |

Positive control compounds are not considered to have “no-takes” regardless of the number of “no-takes” in the control group. Thus, all survivors on Day B are used in the calculation of TIC for the positive control. Surviving animals are evaluated and recorded on the day of evaluation as “cures” or “no-takes.”

Calculation of Median Survival Time (MedST)

MedST is the median day of death for a test or control group. If deaths are arranged in chronological order of occurrence (assigning to survivors, on the final day of observation, a “day of death” equal to that day), the median day of death is a day selected so that one half of the animals died earlier and the other half died later or survived. If the total number of animals is odd, the median day of death is the day that the middle animal in the chronological arrangement died. If the total number of animals is even, the median is the arithmetical mean of the two middle values. Median survival time is computed on the basis of the entire population and there are no deletion of early deaths or survivors, with the following exception:

C. Computation of MedST from Survivors

If the total number of animals including survivors (N) is even, the MedST (days) (X+Y)/2, where X is the earlier day when the number of survivors is N/2, and Y is the earliest day when the number of survivors (N/2)-1. If N is odd, the MedST (days) is X.

D. Computation of MedST from Mortality Distribution

If the total number of animals including survivors (N) is even, the MedST (days) (X+Y)/2, where X is the earliest day when the cumulative number of deaths is N/2, and Y is the earliest day when the cumulative number of deaths is (N/2)+1. If N is odd, the MedST (days) is X. “Cures” and “no-takes” in systems evaluated by MedST are based upon the day of evaluation. On the day of evaluation any survivor not considered a “no-take” is recorded as a “cure.” Survivors on day of evaluation are recorded as “cures” or “no-takes,” but not eliminated from the calculation.

E. Calculation of Approximate Tumor Weight from Measurement of Tumor Diameters with Vernier Calipers

The use of diameter measurements (with Vernier calipers) for estimating treatment effectiveness on local tumor size permits retention of the animals for lifespan observations. When the tumor is implanted sc, tumor weight is estimated from tumor diameter measurements as follows. The resultant local tumor is considered a prolate ellipsoid with one long axis and two short axes. The two short axes are assumed to be equal. The longest diameter (length) and the shortest diameter (width) are measured with Vernier calipers. Assuming specific gravity is approximately 1.0, and Pi is about 3, the mass (in mg) is calculated by multiplying the length of the tumor by the width squared and dividing the product by two. Thus,

$$Tumor weight ( mg ) = \frac{length ( mm ) \times ( width [ mm ] ) 2}{2} or \frac{L \times ( W ) 2}{2}$$

The reporting of tumor weights calculated in this way is acceptable inasmuch as the assumptions result in as much accuracy as the experimental method warrants.

### F. Calculation of Tumor Diameters

The effects of a drug on the local tumor diameter may be reported directly as tumor diameters without conversion to tumor weight. To assess tumor inhibition by comparing the tumor diameters of treated animals with the tumor diameters of control animals, the three diameters of a tumor are averaged (the long axis and the two short axes). A tumor diameter T/C of 75% or less indicates activity and a T/C of 75% is approximately equivalent to a tumor weight T/C of 42%.

G. Calculation of Mean Tumor Weight from Individual Excised Tumors

The mean tumor weight is defined as the sum of the weights of individual excised tumors divided by the number of tumors. This calculation is modified according to the rules listed below regarding “no-takes.” Small tumors weighing 39 mg or less in control mice or 99 mg or less in control rats, are regarded as “no-takes” and eliminated from the computations. In treated groups, such tumors are defined as “no-takes” or as true drug inhibitions according to the following rules:

| Percent of small tumors | Percent of “no-takes” | |
|---|---|---|
| in treated group | in control group | Action |
| ≦17 | Any percent | no-take; not used in |
| | | calculations |
| 18-39 | <10 | drug inhibition; use in |
| | | calculations |
| | ≧10 | no-takes; not used in |
| | | calculations |
| ≧40 | <15 | drug inhibition; use in |
| | | calculations |
| | ≧15 | Code all nontoxic |
| | | tests “33” |

Positive control compounds are not considered to have “no-takes” regardless of the number of “no-takes” in the control group. Thus, the tumor weights of all surviving animals are used in the calculation of T/C for the positive control (T/C defined above) SDs of the mean control tumor weight are computed the factors in a table designed to estimate SD using the estimating factor for SD given the range (difference between highest and lowest observation) (Biometrik Tables for Statisticians Pearson E S & Hartley H G eds. Cambridge Press, vol. 1, table 22, p. 165).

II. Specific Tumor Models

### A. Lymphoid Leukemia L1210

Summary: Ascitic fluid from donor mouse is transferred into recipient BDF1 or CDF1 mice. Treatment begins 24 hours after implant. Results are expressed as a percentage of control survival time. The key parameter is mean survival time. Origin of tumor line: induced in 1948 in spleen and lymph nodes of mice by painting skin with MCA (J Natl Cancer Inst. 13:1328 (1953)).

| | |
|---|---|
| Animals | One sex used for all test and control animals |
| | in one experiment. |
| Tumor Transfer | Inject ip, 0.1 ml of diluted ascitic fluid containing |
| | 10⁵ cells |
| Propagation | DBA/2 mice (or BDF1 or CDF1 for one generation). |
| Time of Transfer | Day 6 or 7 |
| Testing | BDF1 (C57BL/6 × DBA/2) or CDF1 |
| | (BALB/c × DBA/2) |
| Time of Transfer | Day 6 or 7 |
| Weight | Within a 3-g range, minimum weight of 18 g for |
| | males and 17 g for females. |
| Exp Size (n) | 6/group; No. of control groups varies according to |
| | number of test groups. |

### Testing Schedule

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with |
| | therapeutic composition. Typically, mice receive 1 μg of the test |
| | composition in 0.5 ml saline. Controls receive saline alone. |
| | Treatment is one dose/week. Any surviving mice are sacrificed |
| | after 4 wks of therapy. |
| 5 | Weigh animals and record. |
| 20 | If there are no survivors except those treated with positive |
| | control compound, evaluate |
| 30 | Kill all survivors and evaluate experiment. |

Quality Control: Acceptable control survival time is 8-10 days. Positive control compound is 5-fluorouracil; single dose is 200 mg/kg/injection, intermittent dose is 60 mg/kg/injection, and chronic dose is 20 mg/kg/injection. Ratio of tumor to control (T/C) lower limit for positive control compound is 135%.
Evaluation: Compute mean animal weight on Days 1 and 5, and at the completion of testing compute T/C for all test groups with >65% survivors on Day 5. A T/C value 85% indicates a toxic test. An initial T/C 125% is considered necessary to demonstrate activity. A reproduced T/C 125% is considered worthy of further study. For confirmed activity a composition should have two multi-dose assays that produce a T/C 125%.

### B. Lymphocytic Leukemia P388

Summary: Ascitic fluid from donor mouse is implanted in recipient BDF1 or CDF1 mice. Treatment begins 24 hours after implant. Results are expressed as a percentage of control survival time. The key parameter is MedST. Origin of tumor line: induced in 1955 in a DBA/2 mouse by painting with MCA (Scientific Proceedings, Pathologists and Bacteriologists 33:603 (1957)).

| | |
|---|---|
| Animals | One sex used for all test and control animals in |
| | one experiment. |
| Tumor Transfer | Inject ip, 0.1 ml of diluted ascitic fluid containing |
| | 10⁶ cells |
| Propagation | DBA/2 mice (or BDF1 or CDF1 for one generation). |
| Time of Transfer | Day 7 |
| Testing | BDF1 (C57BL/6 × DBA/2) or CDF1 |
| Time of Transfer | (BALB/c × DBA/2) Day 6 or 7 |
| Weight | Within a 3-g range, minimum weight of 18 g for males |
| | and 17 g for females. |
| Exp Size (n) | 6/group; No. of control groups varies according to |
| | number of test groups. |

### Testing Schedule

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with therapeutic |
| | composition. Typically, mice receive 1 μg of the test composition |
| | in 0.5 ml saline. Controls receive saline alone. Treatment is one |
| | dose/week. Any surviving mice are sacrificed after 4 wks of |
| | therapy. |
| 5 | Weigh animals and record. |
| 20 | If there are no survivors except those treated with positive |
| | control compound, evaluate |
| 30 | Kill all survivors and evaluate experiment. |

Acceptable MedST is 9-14 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.
Quality Control: Acceptable MedST is 9-14 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135%. Check control deaths, no takes, etc.
Evaluation: Compute mean animal weight on Days 1 and 5, and at the completion of testing compute T/C for all test groups with >65% survivors on Day 5. A T/C value of 85% indicates a toxic test. An initial T/C of 125% is considered necessary to demonstrate activity. A reproduced T/C 125% is considered worthy of further study. For confirmed activity a composition should have two multi-dose assays that produce a T/C 125%.

### C. Melanotic Melanoma B16

Summary: Tumor homogenate is implanted ip or sc in BDF1 mice. Treatment begins 24 hours after either ip or sc implant or is delayed until an sc tumor of specified size (usually approximately 400 mg) can be palpated. Results expressed as a percentage of control survival time. The key parameter is mean survival time. Origin of tumor line: arose spontaneously in 1954 on the skin at the base of the ear in a C57BL/6 mouse (Handbook on Genetically Standardized Jax Mice. Jackson Memorial Laboratory, Bar Harbor, Me., 1962. See also Ann NY Acad Sci 100, Parts 1 and 2, (1963)).

| | |
|---|---|
| Animals | One sex used for all test and control animals in |
| | one experiment. |
| Propagation Strain | C57BL/6 mice |
| Tumor Transfer | Implant fragment sc by trochar or 12-g needle or |
| | tumor homogenate* every 10-14 days into axillary |
| | region with puncture in inguinal region. |
| Testing Strain | BDF1 (C57BL/6 × DBA/2) |
| Time of Transfer | Excise sc tumor on Day 10-14 from donor mice and |
| | implant as above |
| Weight | Within a 3-g range, minimum weight of 18 g for |
| | males and 17 g for females. |
| Exp Size (n) | 10/group; No. of control groups varies according |
| | to number of test groups. |

*Tumor homogenate: Mix 1 g or tumor with 10 ml of cold balanced salt solution, homogenize, and implant 0.5 ml of tumor homogenate ip or sc. Fragment: A 25-mg fragment may be implanted sc.

### Testing Schedule

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with |
| | therapeutic composition. Typically, mice receive 1 μg of the |
| | test composition in 0.5 ml saline. Controls receive saline alone. |
| | Treatment is one dose/week. Any surviving mice are |
| | sacrificed after 8 wks of therapy. |
| 5 | Weigh animals and record. |
| 60 | Kill all survivors and evaluate experiment. |

Quality Control: Acceptable control survival time is 14-22 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.
Evaluation: Compute mean animal weight on Days 1 and 5, and at the completion of testing compute T/C for all test groups with >65% survivors on Day 5. A T/C value of 85% indicates a toxic test. An initial T/C of 125% is considered necessary to demonstrate activity. A reproduced T/C 125% is considered worthy of further study. For confirmed activity a composition should have two multi-dose assays that produce a T/C 125%.
Metastasis after IV Injection of Tumor Cells

10⁵ B16 melanoma cells in 0.3 ml saline are injected intravenously in C57BL/6 mice. The mice are treated intravenously with 1 g of the composition being tested in 0.5 ml saline. Controls receive saline alone. Mice sacrificed after 4 weeks of therapy, the lungs are removed and metastases are enumerated.

### C. 3LL Lewis Lung Carcinoma

Summary: Tumor may be implanted sc as a 2-4 mm fragment, or im as a 2×10⁶-cell inoculum. Treatment begins 24 hours after implant or is delayed until a tumor of specified size (usually approximately 400 mg) can be palpated. Origin of tumor line: arose spontaneously in 1951 as carcinoma of the lung in a C57BL/6 mouse Cancer Res 15:39, (1955)). See also Malave I et al., J. Natl. Canc. Inst. 62:83-88 (1979).

| | |
|---|---|
| Animals | One sex used for all test and control animals |
| | in one experiment. |
| Propagation Strain | C57BL/6 mice |
| Tumor Transfer | Inject cells im in hind leg or implant fragment sc in |
| | axillary region with puncture in inguinal region. |
| | Transfer on day 12-14 |
| Testing Strain | BDF1 (C57BL/6 × DBA/2) or C3H mice |
| Time of Transfer | Same as above |
| Weight | Within a 3-g range, minimum weight of 18 g for |
| | males and 17 g for females. |
| Exp Size (n) | 6/group for sc implant, or 10/group for im implant.; |
| | No. of control groups varies according to number |
| | of test groups. |

### Testing Schedule

| DAY | PROCEDURE |
|---|---|
| 0 | Implant tumor. Prepare materials. Run positive control in |
| | every odd-numbered experiment. Record survivors daily. |
| 1 | Weigh and randomize animals. Begin treatment with |
| | therapeutic composition. Typically, mice receive 1 μg of the |
| | test composition in 0.5 ml saline. Controls receive saline |
| | alone. Treatment is one dose/week. Any surviving mice are |
| | sacrificed after 4 wks of therapy. |
| 5 | Weigh animals and record. |
| Final day | Kill all survivors and evaluate experiment. |

Quality Control: Acceptable im tumor weight on Day 12 is 500-2500 mg. Acceptable im tumor MedST is 18-28 days. Positive control compound is cyclophosphamide: 20 mg/kg/injection, qd, Days 1-11. Check control deaths, no takes, etc.
Evaluation: Compute mean animal weight when appropriate, and at the completion of testing compute T/C for all test groups. When the parameter is tumor weight, a reproducible T/C of 42% is considered necessary to demonstrate activity. When the parameter is survival time, a reproducible T/C of 125% is considered necessary to demonstrate activity. For confirmed activity a composition must have two multi-dose assays

### D. 3LL Lewis Lung Carcinoma Metastasis Model

This model has been utilized by a number of investigators. See, for example, Gorelik, E. et al., J. Natl. Canc. Inst. 65:1257-1264 (1980); Gorelik, E. et al., Rec. Results Canc. Res. 75:20-28 (1980); Isakov, N. et al., Invasion Metas. 2:12-32 (1982) Talmadge J E et al., J. Nat'l. Canc. Inst. 69:975-980 (1982); Hilgard, P. et al., Br. J. Cancer 35:78-86 (1977)).

Mice: male C57BL/6 mice, 2-3 months old. Tumor: The 3LL Lewis Lung Carcinoma was maintained by sc transfers in C57BL/6 mice. Following sc, im or intra-footpad transplantation, this tumor produces metastases, preferentially in the lungs. Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected into the right hind foot pads of C57BL/6 mice. The day of tumor appearance and the diameters of established tumors are measured by caliper every two days.

In experiments involving tumor excision, mice with tumors 8-10 mm in diameter are divided into two groups. In one group, legs with tumors are amputated after ligation above the knee joints. Mice in the second group are left intact as nonamputated tumor-bearing controls. Amputation of a tumor-free leg in a tumor-bearing mouse has no known effect on subsequent metastasis, ruling out possible effects of anesthesia, stress or surgery. Surgery is performed under Nembutal anesthesia (60 mg veterinary Nembutal per kg body weight).

### Determination of Metastasis Spread and Growth

Mice are killed 10-14 days after amputation. Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ^{125I}dUrd into lung cells (Thakur M L et al., J. Lab. Clin. Med. 89:217-228 (1977)). Ten days following tumor amputation, 25 mg of ¹²⁵IdUrd is inoculated into the peritoneums of tumor-bearing (and, if used, tumor-resected mice. After 30 min, mice are given 1 mCi of ¹²⁵IdUrd. One day later, lungs and spleens are removed and weighed, and a degree of ¹²⁵IdUrd incorporation is measured using a gamma counter.

Statistics: Values representing the incidence of metastases and their growth in the lungs of tumor-bearing mice are not normally distributed. Therefore, non-parametric statistics such as the Mann-Whitney U-Test may be used for analysis.

Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of large doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 10⁶ 3LL cells. Amputation of tumors produced following inoculation of 10⁵ tumor cells dramatically accelerated metastatic growth. These results were in accord with the survival of mice after excision of local tumors. The phenomenon of acceleration of metastatic growth following excision of local tumors had been observed by other investigators. The growth rate and incidence of pulmonary metastasis were highest in mice inoculated with the lowest doses (3×10⁴-10⁵ of tumor cells) and characterized also by the longest latency periods before local tumor appearance. Immunosuppression accelerated metastatic growth, though nonimmunologic mechanisms participate in the control exerted by the local tumor on lung metastasis development. These observations have implications for the prognosis of patients who undergo cancer surgery.

### E. Walker Carcinosarcoma 256

Summary: Tumor may be implanted sc in the axillary region as a 2-6 mm fragment im in the thigh as a 0.2-ml inoculum of tumor homogenate containing 10⁶ viable cells, or ip as a 0.1-ml suspension containing 10⁶ viable cells. Origin of tumor line: arose spontaneously in 1928 in the region of the mammary gland of a pregnant albino rat (J Natl Cancer Inst 13:1356, (1953)).

| | |
|---|---|
| Animals | One sex used for all test and control animals in |
| | one experiment. |
| Propagation Strain | Random-bred albino Sprague-Dawley rats |
| Tumor Transfer | S.C. fragment implant is by trochar or 12-g needle |
| | into axillary region withpuncture in inguinal area. |
| | I.m. implant is with 0.2 ml of tumor homogenate |
| | (containing 10⁶ viable cells) into the |
| | thigh. I.p. implant is with 0.1 ml suspension |
| | (containing 10⁶ viable cells) |
| | Day 7 for im or ip implant; Days 11-13 for |
| | sc implant |
| Testing Strain | Fischer 344 rats or random-bred albino rats |
| Time of Transfer | Same as above |
| Weight | 50-70 g (maximum of 10-g weight range within |
| | each experiment) |
| Exp Size (n) | 6/roup; No. of control groups varies according to |
| | number of test groups. |

| Test | Prepare drug | Administer | Weigh animals | Evaluate on |
|---|---|---|---|---|
| system | on day: | drug on days: | on days | days |
| 5WA16 | 2 | 3-6 | 3 and 7 | 7 |
| 5WA12 | 0 | 1-5 | 1 and 5 | 10-14 |
| 5WA31 | 0 | 1-9 | 1 and 5 | 30 |

In addition the following general schedule is followed

| DAY | PROCEDURE |
|---|---|
| 0 | Implant tumor. Prepare materials. Run positive control in |
| | every odd-numbered experiment. Record survivors daily. |
| 1 | Weigh and randomize animals. Begin treatment with |
| | therapeutic composition. Typically, mice receive 1 μg |
| | of the test composition in 0.5 ml saline. Controls receive |
| | saline alone. Treatment is one dose/week. Any |
| | surviving mice are sacrificed after 4 wks of therapy. |
| Final day | Kill all survivors and evaluate experiment. |

Quality Control: Acceptable i.m. tumor weight or survival time for the above three test systems are: 5WA16: 3-12 g.; 5WA12: 3-12 g.; 5WA31 or 5WA21: 5-9 days.
Evaluation: Compute mean animal weight when appropriate, and at the completion of testing compute T/C for all test groups. When the parameter is tumor weight, a reproducible T/C 42% is considered necessary to demonstrate activity. When the parameter is survival time, a reproducible T/C 125% is considered necessary to demonstrate activity. For confirmed activity
F. A20 lymphoma

10⁶ murine A20 lymphoma cells in 0.3 ml saline are injected subcutaneously in Balb/c mice. Tumor growth is monitored daily by physical measurement of tumor size and calculation of total tumor volume. After 4 weeks of therapy the mice are sacrificed. Having now generally described the invention, the same will be more readily understood through reference to the following examples which are provided by way of illustration, and are not intended to be limiting of the present invention, unless specified.

### Example 1

Examples 1 and 2 are cumulative disclosures from U.S. Ser. No. 09/751,708, U.S. 60/438,686, U.S. 60/415,310, U.S. 60/406,750, U.S. 60/415,400, U.S. 60/406,697, U.S. 60/389,366, U.S. 60/378,988, U.S. Ser. No. 09/870,759 which are incorporated by reference and their references in their entirety.

### Sickled Erythrocytes as Carriers of Tumoricidal Agents.

Sickled erythrocytes are known to be more adherent to microvascular endothelium than normal erythrocytes and to adhere to a greater extent under conditions of local hypoxia and acidosis. The primary pathologic defect in sickle cell disease is the abnormal tendency of hemoglobin S to polymerize under hypoxic conditions. The polymerization of deoxygenated hemoglobin S results in a distortion of the shape of the red cell and marked decrease in its deformability. These rigid cells are responsible for the vaso-occlusive phenomena which are the hallmark of the disease.

Sickle red cells adhere to the microvascular endothelium for the following reasons: Sickled cells have abnormally increased expression of α₄β₁ integrin and CD36. Activation of platelets releases thrombospondin, which act as a bridging molecule by binding to a surface molecule, CD36, on an endothelial cell and to CD36 or sulfated glycans on a sickle reticulocyte. Inflammatory cytokines induce the expression of vascular-cell adhesion molecule 1 (VCAM-1) on endothelial cells. This adhesive molecule binds directly to the α₄β₁ integrin on the sickle reticulocyte.

In the oxygenated state, the extent of sickle cell adhesion is density-class dependent: reticulocytes and young discocytes (SS1) greater than discocytes (SS2) greater than irreversible sickle cells and unsicklable dense discocytes (SS4). Hypoxemic conditions have no effect on adherence of normal erythrocytes but sickle erythrocyte adherence to endothelial cells is increased significantly. The least dense sickle erythrocytes containing CD36 and VLA-4+ expressing reticulocytes are especially involved in hypoxia sensitive adherence. Selective secondary trapping of SS4 (dense cells) occurs in post capillary venules where deformable SS cells are preferentially adherent. Vaso-occlusion is induced by a combination of precapillarly obstruction, adhesion in post capillary venules, and secondary trapping of dense erythrocytes. This induces local hypoxia leading to increased polymerization of hemoglobin S and rigidity of SS erythrocytes. In this way the obstruction is multiplied and extended to nearby vessels.

In the present invention, sickled erythrocytes are used to carry tumoricidal agents into the microvasculature of tumors. Sickle cell trait cells are preferred since they are normal under physiologic conditions but sickle and become adhesive in the acidotic and/or hypoxemic tumor microvasculature. Tumoricidal agents introduced into and carried by sickled erythrocytes include oncolytic viruses including but not limited to herpes simplex, adenoviruses, vaccinia, Newcastle Disease virus, autonomous parvoviruses, In addition, the adenovirus encoding thymidine kinase is transfected into tumor cells that are then susceptible to lysis ganciclovir. Various oncolytic and tumor specific viruses with tumor specificity used to transfect sickle cells are described in Kirn, D. et al., Nat. Med. 7:781-7 (2001).

In addition the sickled erythrocyte carry nucleic acids encoding tumoricidal agents including but not limited to C. perfringens exotoxin, pertussis toxin, verotoxins, pseudomonas exotoxins and superantigens, perforin, granzyme B, complement components (membrane attack complex), oxidized LDL, tumor specific antibodies alone or fused to toxins including but not limited to superantigens, Pseudomonas exotoxins, ricin, clostridia toxin. The nucleic acid encodes a hemolysin such as but not limited to E. coli hemolysin or staphylococcal alpha hemolysin. The sickled cell can also contain anaerobic bacterial spores such as clostridia species which can grow selectively in hypoxemic tissues. The sickled erythrocyte also carries phage displays, exosomes, and sickle cell vesicles, sec vesicles expressing tumor toxins or superantigens. The toxins may be fusion proteins of toxins with ligands expressed on tumor vasculature or tumor such a EGF, inactivated factor VIII or antibodies specific for a wide variety of tumor antigens well known in the art.

The nucleic acids encoding these toxins and oncolytic and tumor specific viruses are placed under the promoter of the heat sensitive global operator (Example 69). When entering the hypoxic tumor, sickled erythrocyte adhere to the tumor vasculature. In the hypoxemic environment of the tumor, the hypoxia sensitive global promoter is activated and induces the production lytic viruses and toxins. Sickled cells are disrupted and lyse releasing lytic virus and toxin into the hypoxic tumor. As the tumor site becomes more hypoxic, VCAM-1 and p-selectin expression on tumor endothelium are upregulated trapping more circulating sickled cells in the tumor microcirculation to undergo lysis with release of tumoricidal products into the tumor area.

The sickled cell is transfected preferably with the oncolytic viruses and toxins given above at a stage preferably before it is enucleated (Examples 1, 60, 69). Nucleated sickle reticulocytes are the preferred cell for transfection although enucleated sickled cells will also work (Example 69 of PCT/US03/14381) Anaerobic bacterial spores such clostridia are transfected into the sickled erythrocytes by endocytosis or electroporation (Schrier S. Meth. Enzymol. 149: 261-271 (1987); Tsong T Y Meth. Enzymol. 149-259 (1987)). They are also introduced into sickle erythrocytes that have been lysed under hypotonic conditions and the membranes annealed with encapsulation of the anaerobic spores (Example 69).

Erythrocytes from subjects with sickle trait are preferred because these red cells are functionally and structurally normal in the circulation but are activated to sickle in the hypoxic tumor vasculature. Here they assume the sickled configuration, adhere to the endothelium of the tumor microcirculation and obstruct microvasculature in a manner similar to the homozygous SS erythrocytes.

The sickled erythrocytes are administered parenterally by injection or infusion in a therapeutically effective amount of cells. This encompasses a volume of 1-25 cc of packed cells administered i.v. over a one hour period. These cells are used in protocols given in Example 14-16, 18-23, 66 of PCT/US03/14381.

### Sickled Erythrocytes as Gene Carriers

Erythrocytes from patients with sickle cell anemia contain a high percentage of SS hemoglobin which under conditions of deoxygenation aggregate followed by the growth and alignment of fibers transforming the cell into a classic sickle shape. Retardation of the transit time of sickled erythrocytes results in vaso-occlusion. SS red blood cells have an adherent surface and attach more readily than normal cells to monolayers of cultured tumor endothelial cells. Reticulocytes from patients with SS disease have on their surface the integrin complex α4β1 which binds to both fibronectin and VCAM-1, a molecule expressed on the surface of tumor endothelial cells particularly after activation by inflammatory cytokines such as TNF, interleukins and lipid-mediated agonists (prostacyclins). Activated tumor endothelial cells are typically procoagulant. Similar molecules are upregulated on the neovasculature of tumors. In addition, upregulation of the adhesive and hemostatic properties of tumor endothelial cells are induced by viruses, such as herpes virus and Sendai virus. Sickled erythrocytes lack structural malleability and aggregate in the small tortuous microvasculature and sinusoids of tumors. In addition, the relative hypoxemia of the interior of tumors induces aggregation of sickled erythrocytes in tumor microvasculature. Hence, sickled erythrocytes with their proclivity to aggregate and bind to the tumor endothelium are ideal carriers of therapeutic genes to tumor cells.

Red blood cell mediated transfection is used to introduce various nucleic acids into the sickled erythrocytes. The extremely plastic structure of the erythrocyte and the ability to remove its cytoplasmic contents and reseal the plasma membranes enable the entrapment of different macromolecules within the so-called hemoglobin free “ghost.” Combining these ghosts and a fusogen such as polyethylene glycol has permitted the introduction of a variety of macromolecules into mammalian cells (Wiberg, F C et al., Nucleic Acid Res. 11: 7287-7289 (1983); Wiberg, F C et al., Mol. Cell. Biol. 6: 653-658 (1986); Wiberg, F C et al., Exp. Cell. Res. 173: 218-227 (1987)). Both transient and stable expressions of introduced DNA are achieved by this method. Sickled cells can also be transfected with a nucleic acid of choice e.g., apolipoproteins, RGD in the nucleated prereticulocyte phase (e.g. proerythroblast or normoblast stage) by methods given in Example 1 of PCT/US03/14381. Sickled erythrocytes transfected with nucleic acids encoding a SAg and/or carbohydrate modifying enzyme to induce expression of the α-Gal epitope, apolipoproteins, RGD and/or any construct described herein. Nucleic acids encoding additional polypeptides alone or together with SAg as described in Tables I and II of PCT/US03/14381 including but not limited to angiostatin, apolipoproteins, RGD, streptococcal or staphylococcal hyaluronidase, chemokines, chemoattractants and Staphylococcal protein A are transfected into and expressed by sickled erythrocytes. These sickled cell transfectants are administered parenterally and localize to tumor neovascular endothelial sites where they induce a anti-tumor response. The methods of in vivo transfection of tumor cells are given in the Examples 17 of PCT/US03/14381. Protocols for use of these transfectants in the induction of antitumor immune response are described in Examples 14, 15, 16, 18-23, 31 of PCT/US03/14381. Superantigen nucleic acids together with nucleic acids encoding either apo(a), apoB and apoE4 are also transfected into nucleated sickled erythrocytes (e.g., proerythroblast or normoblast phase) by methods given in Examples 1 and 6 of PCT/US03/14381. The integrin ligand RGD nucleic acids are transfected into tumor cells or sickled cells to facilitate the localization of the transfected tumor cells and sickled cells to integrins expressed in the tumor neovasculature in vivo (see Example 6). Alternatively, the sickled erythrocytes or tumor cells acquire the apolipoprotein or oxyLDL by coculture with liposomes which express the apolipoprotein or oxyLDL (see Section 7 & Example 5 of PCT/US03/14381).

These tumor cells or sickle cell transfectants are administered parenterally and are capable of trafficking to tumor microvasculature wherein they bind to apolipoprotein and scavenger receptors on endothelial cells and macrophages. The transfectants are phagocytosed by macrophages cells and induce endothelial cell apoptosis. SAgs expressed on the tumor cells and sickle cells also induce a local T cell inflammatory anti-tumor response which envelops the neighboring tumor cells.

### Methods for Preparing Sickled Erythrocytes for Use as Carriers Tumoricidal Agents

The sickled cells are obtained from patients with sickle cell anemia or sickle cell trait. The type of sickle cell disease may be hemoglobin SS, hemoglobin SC, or the combination of hemoglobin SS and β-thalassemia. To determine compatibility of donor sickled erythrocytes with recipient erythrocytes, the donor cells are ABO typed and matched. The tendency of these red cells to adhere to cultured endothelial cells is assayed in vitro by the method of Hebbel R P et al., New Eng. J. Med. 302: 992-995 (1980). The sickled cells are harvested, transfected with appropriate oncolytic or tumor specific viruses, toxins or anaerobic bacteria in vitro by methods given in Example 1. Fifty to 250 cc of transfected sickled erythrocytes are infused intravenously over 1-2 hours. The procedure is repeated two to three times weekly for two to four weeks. Responsive patients are retreated on a similar schedule if tumor reappears. The patient's vital signs are monitored every 10 minutes during the infusion, then every hour for the next 4 hours and Q4-6 hours thereafter.

Infection of nucleated erythrocytes by oncolytic or tumor specific viruses: This is carried out by the method of Muhlemann, O., Akusjarvi, G., in Adenovirus Methods and Protocols WSM Wold, editor, Humana Press, Totowa, N.J. (1999). Essential steps are given below. Transfection of nucleated sickled cells with various plasmid DNAs described in section 66 of PCT/US03/14381 is carried out as in Examples 1 and 60 of PCT/US03/14381.
Infection of sickled cells with adenovirus: Sickled cells are grown in round cell-culture bottles on a magnetic stirrer at 37° C. in MEM spinner cell medium, 5% newborn calf serum, optionally containing 1% penicillin/streptomycin. The cells must be kept in log phase (titer 2-6×10⁵ cells/mL), doubling time approx 24 h.
1. Start with 2-3×10⁹ sickled spinner cells; collect them by centrifugation in sterile 1-L plastic bottles by spinning at 900 g at room temperature for 20 min. (Beckman J6M/E centrifuge, JS-4.2 rotor).
2. Decant medium back into the cell-culture bottle (handle under sterile conditions the medium will be reused later), resuspend cells in 200-300 mL MEM without serum (see Note 1), and transfer to a 1-L cell-culture bottle.
3. Infect cells with approx 10 PFU/cell of adenovirus from a high-titer virus preparation. Leave at 37° C. on a magnetic stirrer for 1 h. Dilute cells to approximately 4×10⁵ cells per mL in a large cell culture bottle with the old MEM medium saved at step 2. Add fresh medium if necessary.
4. Continue incubation at 37° C. for 20-24 h for preparation of late-infected extracts.
   Additional protocols for infecting sickled cells with various lytic viruses or tumor selective viruses are given in Example 60 and in Adenovirus Methods and Protocols WSM Wold, editor, Humana Press, Totowa, N.J. (1999) which is herein incorporated in entirety by reference.
   Preparation of the Hypoxia Responsive Element Promoter of the VEGF Gene Cloning and Sequencing of the Mouse VEGF Promoter Region: The VEGF promoter region is amplified by PCR using genomic DNA isolated from mouse liver, oligonucleotide primers synthesized on the basis of the published DNA sequence (GenBank accession number U41383), and LA Taq DNA polymerase (TaKaRa Biomedicals, Osaka, Japan). The sense and antisense primers are (SEQ ID NO: 69)-1215 (5′-TTTAGAAGATGAACCGTAAGC-CTAG-3′) and (SEQ ID NO: 70)+315 (5′-GATACCTCTTTCGTCTGCTGA-3′), respectively. The PCR conditions are 94° C. for 5 min followed by 30 cycles of 94° C. for 30 s, 68° C. for 3 min, and 72° C. for 7 min. The PCR product, which contained the 5′-flanking sequence encompassing the putative HRE site, the transcription start site, and the 5′-untranslated region, is gel-purified and subcloned into a TA cloning vector prepared from EcoRV-cut pBluescript KS- (Stratagene, La Jolla, Calif.). Several independent clones are sequenced, and a clone is used for additional experiments. Deletion of the HRE site is obtained by digestion with BsaA1, a recognition site of which resides in the middle of the HRE site.
   Luciferase Reporter Plasmid Constructs and Luciferase Assays: The VEGF promoter sequence with or without the HRE site in pBluescript KS- is excised by digestion with the appropriate restriction enzymes, gel-purified, and blunt-ended with T4 DNA polymerase, and the fragment was ligated into SmaI-cut pGL2-Basic vector (Promega, Madison, Wis.), yielding plasmids pGLV(HRE)Luc or pGLV(AHRE)Luc, respectively. The orientation of the insert is verified by restriction enzyme analysis. Transient transfection was carried out using Lipofectin (Life Technologies, Inc., Gaithersburg, Md.). As a control for transfection efficiency, pRL-CMV vector (Promega) is cotransfected with test plasmids. pGL2-control vector (Promega) was used as a positive control. Luciferase activity in cell extracts is assayed 48 h after transfection according to the Dual-Luciferase reporter assay system protocols (Promega) using a luminometer (model TD-20/20; Turner Designs, Sunnyvale, Calif.).
   Construction of Retroviral Vectors: Retroviral vector LXSN (provided by Dr. A. D. Miller, Fred Hutchinson Cancer Research Center, Seattle, Wash.) is modified as follows to create a multicloning site. The retroviral vector is digested with EcoRI and XhoI and blunt-ended with T4 DNA polymerase. A SacI/KpnI fragment of pBluescript SK- that is blunt-ended with T4 DNA polymerase is ligated to this vector. This procedure yields retroviral vector LXSN(BA), which has a multicloning site between the Betel site and the Appall site of pBluescript KS-. A retroviral vector harboring the VEGF promoter sequence, HSV-TK gene or GFP gene, and SV40pA, all of which are located in a reverse orientation of LTR, is obtained as follows. A SV40pA fragment is prepared by digestion of Pezos (Invitrogen Corp., Carlsbad, Calif.) with Accl and BamHI. The fragment is gel-purified, blunt-ended with T4 DNA polymerase, and ligated into Bxt/XI-cut and blunt-ended LXSN(BA), yielding a LXSN(BA)/pA vector. The VEGF promoter region with or without the HRE site in pBluescript KS-is excised with EcoRI and San and ligated into EcoRI/SalI-cut LXSN(BA)/pA, generating vectors LV(HRE) and LV(AHRE), respectively. The GFP or HSV-TK gene or any other gene given in section 66 is cloned into the NotI site of these vectors via NotI linkers. The orientation of the inserts is verified by restriction enzyme analysis. The retroviral vectors generated by this procedure are termed LV(HRE)GFP, LV(HRE)TK, and LV(ΔHRE)TK.
   Plasmid Transfection and Retrovirus Infection: All cells are transfected with the: plasmids using Lipofectin. The retroviruses harboring LV(HRE)GFP or LV(HRE)TK are generated by a φ2 packaging cell line. All cells were infected with the retroviruses in the presence of 8 μg/ml polybrene (Aldrich Chemical Co., Inc., Milwaukee, Wis.). The cells are cultured in the presence of 400 μg/ml G418 (Life Technologies, Inc., Grand Island, N.Y.) to select for cells that expressed vector-derived genes.

### Evaluation of GFP Expression and Vascular in Cryosections of Tumors:

Cells: (2×10⁵) transfected with LV(HRE)GFP are subcutaneously injected into the flank of gynogenic C57BL/6 mice (Nippon SLC, Hamada's, Japan). Ten days after the injection, tumors are surgically removed and frozen in OCT compound. Cryostat sections are fixed with cold acetone and washed with DPBS, and endogenous peroxides is blocked with 3% hydrogen peroxide in methanol for 10 min. The samples are washed three times with DPBS and incubated with DPBS containing 10% normal goat serum for 60 min to block nonspecific binding sites. They are then incubated with rat ant mouse CD31 antibody (Harlingen, San Diego, Calif.). Sections are washed with DPBS and incubated with TRITC-conjugated goat antiriot IgG. After extensive washings with DPBS, samples are mounted in 50% glycerol in DPBS containing 1 mg/ml/phenylenediamine. The fluorescence emitted from GFP and TRITC is observed under a confocal laser microscope (Fluoview; Olympus, Tokyo, Japan). Alternatively, cells are subjected to hypoxia for 16 h followed by exposure to GCV for 24 h in air, and the cell number was determined 2 days after the treatment.
In Vivo Experiments: Cells (2.5×10⁵) retrovirally transduced with LV(HRE)TK or LV(HRE) are s.c. injected into 6-week-old female C57BL/6 mice. Ten days after the inoculation, GCV diluted in DPBS is i.p. injected at a concentration of 30 mg/kg twice daily at 8-h intervals for 5 days. DPBS alone is injected into control mice. Tumor growth is monitored by caliper measurement of two diameters at right angles, and the tumor mass is estimated from the equation volume=0.5×a×b², where a and b are the larger and smaller diameters, respectively.

### Example 2

### Vesicles from Sickled Erythrocytes

Vesicles from sickled erythrocytes are shed from the parent cells. They contain membrane phospholipids which are similar to the parent cells but are depleted of spectrin. They also demonstrate that a shortened Russell's viper venom clotting time by 55% to 70% of control values and become more rigid under acid pH conditions. Rigid sickle cell vesicles induce hypercoagulability, are unable to pass through the splenic circulation from which they are rapidly removed. Sickled erythrocytes are transfected in the nucleated prereticulocyte phase with superantigen and apolipoprotein nucleic acids as well as RGD nucleic acids. Nucleic acids encoding additional polypeptides alone or together with SAg as described in Tables I and II are transfected into and expressed by sickled erythrocytes. Any of the immature or mature sickled erythrocytes and their shed vesicles expressing the molecules given in Tables I and II are capable of localizing to tumor microvascular sites where they bind to apolipoprotein receptors and induce an anti-tumor effect. Because of their adhesive and hypercoagulable properties as well as their rigid structure, these sickled cell vesicles expressing superantigen and apolipoproteins are especially useful for targeting the tumor microvascular endothelium and producing a prothrombotic, inflammatory anti tumor effect. Sickled erythrocytes and their vesicles are capable of acquiring oxyLDL via fusion with oxyLDL containing liposomes as in Example 5. The resulting sickle cell or liposome expresses oxyLDL alone or together with SAg. Binding of oxyLDL to the SREC receptor on tumor microvascular endothelial cells induces apoptosis and simultaneous superantigen deposition produces a potent T cell anti-tumor effect.

Vesicles are prepared and isolated as follows: Blood is obtained from patients with homozygous sickle cell anaemia. The PCV range is 20-30%, reticulocyte range is 8-27%, fetal hemoglobin range is 25-13% and endogenous level of ISCs is 2-8%. Blood is collected in heparin and the red cells are separated by centrifugation and washed three times with 09% saline. Cells are incubated at 37° C. and 10% PCV in Krebs-Ringer solutions in which the normal bicarbonate buffer is replaced by 20 mM Hepes-NaOH buffer and which contains either 1 mM CaCl₂ or 1 mM EGTA. All solutions contain penicillin (200 U/ml) and streptomycin sulphate (100 μg/ml). Control samples of normal erythrocytes are incubated in parallel with the sickle cells. Incubations of 10 ml aliquots are conducted in either 100% N₂ or in room air for various periods in a shaking water bath (100 oscillations per mm). N₂ overlaying is obtained by allowing specimens to equilibrate for 45 mm in a sealed glove box (Gallenkamp) which was flushed with 100% N₂. Residual oxygen tension in the sealed box was less than 1 mmHg. The percentage of irreversibly sickled cells is determined by counting. 1000 cells after oxygenation in room air for 30 mm and fixation in buffered saline (130 mM NaCl, 20 mM sodium phosphate, pH 74) containing 2% glutaraldehyde. Cells whose length is greater than twice the width and which possessed one or more pointed extremities under oxygenated conditions are considered to be irreversibly sickled.

After various periods of incubation, cells are sedimented at 500 g for 5 mm and microvesicles are isolated from the supernatant solution by centrifugation at 15,000 g for 15 mm. The microvesicles form a firm bright red pellet sometimes overlain by a pink, flocculent pellet of ghosts (in those cases where lysis was evident) which is removed by aspiration. Quantitation of microvesicles is achieved by resuspension of the red pellet in 1 ml of 0.5% Triton X100 followed by measurement of the optical density of the clear solution at 550 nm. Optical density measurements at 550 nm give results that are relatively the same as measurements of phospholipid and cholesterol content in the microvesicles. Cell lysis is determined by measurement of the optical density at 550 nm of the clear supernatant solution remaining after sedimentation of the microvesicles. Larger samples of microvesicles for biochemical and morphological analysis are prepared from both sickle and normal cells following incubation of up to 100 ml of cell suspension at 37° C. for 24 h in the absence or presence of Ca²⁺. Ghosts are prepared from sickle cells after various periods of incubation. The cells are lysed and the ghosts washed in 10 mM Tris HCl buffer, pH 73, containing 0.2 mM EGTA.

These vesicles are useful as a preventative or therapeutic vaccine.

### Example 3

For human studies, SS erythrocytes or nucleated SS erythrocyte precursors are obtained from patients with homozygous S or sickle thalassemia hemoglobin, hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. Nucleated erythroleukemia cells are obtained from patients with erythroleukemia. The erythrocytes are ABO- and Rh-matched for compatibility with recipients. The cells are optionally incubated with epinephrine 1×10⁻² μM per 10⁸ cells for 2 minutes at 37° C. SS erythroblasts and erythroleukemia cells stably transfected with nucleic acids encoding BCAM/Lu are transfected with oncolytic viruses as described herein. Additional groups of these cell types are rendered drug-resistant by ex vivo exposure to cisplatinum or Adriamycin as described herein. Mature SS cells are loaded with antitumor drugs or oncolytic viruses operative in enucleated SS RBCs as described herein. All cells are optionally irradiated with light before administration to induce a photohemolysis t½ h of 10-60 minutes after intravenous administration. The total amount of antitumor drug administered per treatment with the any of these cell types is in a range of 25-100 mg.

Tumors of any type are susceptible to therapy with these agents. The cells are administered intravenously or intraarterially in a blood vessel perfusing a specific tumor site or organ, e.g. carotid artery, portal vein, femoral artery etc. over the same amount of time required for the infusion of a conventional blood transfusion. The quantity of cells to be administered in any one treatment ranges from one tenth to one half of a full unit of blood. The treatments are generally given every 2-7 days for a total of 1-12 treatments. However, the treatment schedule is flexible and may be given for a longer of shorter duration depending upon the patients' response. All treated patients have histologically confirmed malignant disease including carcinomas, sarcomas, melanomas, lymphomas and leukemias and have failed conventional therapy. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage 1 (localized disease) to Stage 4 (widespread metastases). Patient history is obtained and physical examination performed along with conventional tests of cardiovascular and pulmonary function and appropriate radiologic procedures. Histopathology is obtained to verify malignant disease.

Results: A total of 1011 patients are patients treated, 339 with mature SS cells, 338 with SS progenitor cells and 339 with erythroleukemia cells stably transfected with BCAM/Lu. All cells are stably transfected with or have encapsulated oncolytic virus as described herein and irradiated with light before intravenous administration. The overall number of patients for each tumor type and the results of treatment are summarized in Table 7. Positive tumor responses are observed in as high as 85-95% of the patients with breast, gastrointestinal, lung, prostate, renal and bladder tumors as well as melanoma and neuroblastoma as follows.

Eight hundred and ninety one of 1011 entered with all tumors exhibit objective clinical responses for an overall response rate of 89%. Tumors generally start to diminish and objective remissions are evident after four weeks of therapy. Responses endure for a mean of 36 months.

TABLE 7
SS cells, SS progenitor cells and erythroleukemia cells loaded
with oncogenic virus

| | | | % of Patients |
|---|---|---|---|
| Patients/Tumors | No. | Response | Responding |
| All Patients | 891 | CR + PR | 88 |
| Tumor Type | | | |
| Breast adenocarcinoma | 165 | CR + PR | 90% |
| Gastrointestinal carcinoma | 156 | CR + PR | 90% |
| Lung Carcinoma | 200 | CR + PR | 95% |
| Brain glioma/astrocytoma | 60 | CR + PR | 85% |
| Prostate Carcinoma | 130 | CR + PR | 85% |
| Lymphoma/Leukemia | 61 | CR + PR | 80% |
| Head and Neck Cancer | 82 | CR + PR | 80% |
| Renal and Bladder Cancer | 53 | CR + PR | 95% |
| Melanoma | 67 | CR + PR | 85% |
| Neuroblastoma | 37 | CR + PR | 85% |

Toxicity consists of mild short-lived fever, fatigue and anorexia not requiring treatment. The incidence of side effects (as % of total treatments) are as follows: chills—12; fever—15; pain—6; nausea—3; respiratory—2; headache—2; tachycardia—4; vomiting—4; hypertension—1; hypotension—2; joint pain—3; rash—1; flushing—4; diarrhea—2; itching/hives—1; bloody nose—1; dizziness—<1; cramps—<1; fatigue—<1; feeling faint—<1; twitching—<1; blurred vision—<1; gastritis <1; redness on hand—<1. Fever and chills are the most common side effects observed.

In an additional study, 986 patients are treated, 328 with SS progenitor cells 329 with erythroleukemia cells and 327 with mature SS cells. SS progenitor cells and erythroleukemia cells are rendered resistant to anti-tumor drugs in vitro as described herein and mature SS cells have encapsulated anti-tumor drugs as described herein. The chemotherapeutic agent selected for loaded into cells the one to which the tumor is known to be most sensitive as described herein. All cells are irradiated with light before intravenous administration as described herein.

The overall number of patients for each tumor type and the results of treatment are summarized in Table 8. Positive tumor responses are observed in as high as 85-95% of the patients with breast, gastrointestinal, lung, prostate, renal and bladder tumors as well as melanoma and neuroblastoma.

Eight hundred and seventy of 986 patients entered with all tumors exhibit objective clinical responses for an overall response rate of 89%. Tumors generally start to diminish and objective remissions are evident after three to four weeks of therapy. Responses endure for a mean of 36 months.

TABLE 8
SS cells, SS progenitor cells, erythroleukemia cells loaded with
anti-tumor drugs

| | | | % of Patients |
|---|---|---|---|
| Patients/Tumors | No. | Response | Responding |
| All Patients | 870 | CR + PR | 89 |
| Tumor Type | | | |
| Breast adenocarcinoma | 162 | CR + PR | 90% |
| Gastrointestinal carcinoma | 153 | CR + PR | 90% |
| Lung Carcinoma | 195 | CR + PR | 95% |
| Brain glioma/astrocytoma | 57 | CR + PR | 85% |
| Prostate Carcinoma | 127 | CR + PR | 85% |
| Lymphoma/Leukemia | 61 | CR + PR | 80% |
| Head and Neck Cancer | 80 | CR + PR | 80% |
| Renal and Bladder Cancer | 51 | CR + PR | 95% |
| Melanoma | 63 | CR + PR | 85% |
| Neuroblastoma | 37 | CR + PR | 85% |

Toxicity consists of mild fatigue, anorexia and nausea not requiring treatment. The incidence of side effects (as % of total treatments) are as follows: fatigue—15; nausea—12; anorexia—10; chills—3; fever—1; pain—2; respiratory—2; headache—2; tachycardia—4; vomiting—4; hypertension—2; hypotension—1; joint pain—2; rash—1; flushing—1; diarrhea—4; itching/hives—1; bloody nose—1; dizziness—<1; cramps—<1; feeling faint—<1; twitching—<1; blurred vision—<1; gastritis<1; redness on hand—<1.

### Example 4

### Generation of Self-Replication Alphavirus Transcripts

To construct a replication competent, cytopathic vector, the SINrep5 vector containing the nucleic acids encoding Sindbis virus RNA replicase and the SP6 promoter are used. Replicon transcription plasmid pSINrep5 is shown in FIG. 1 of Bredenbeek P J et al., J. Virol. 67: 6439-6446 (1993). Plasmid numbering begins with the sequence corresponding to the first nucleotide of the Sindbis virus genome RNA sequence. Upstream from the Sindbis virus cDNA is the promoter (hatched box and arrow) for SP6 DNA-dependent RNA polymerase, used for production of RNA transcripts in vitro. Unique restriction sites and their positions in the pSINrep5 sequence, including those which can be used for cloning and expression of heterologous sequences (Cloning) or production of templates for runoff transcription (Run off), are indicated. The position corresponding to the subgenomic mRNA start site (nt 7598) is marked (bold arrow). Also indicated are the regions of the plasmid encoding the ampicillin resistance gene (bla) and the origin of replication (ori). The sequence of the cloning region, located between Sindbis virus nt 7646 and 11394, is (SEQ ID NO: 71) 5′-TCTAGACGCGTAGATCT CACGTGAGCATGCAGGCCTTGGG-3′.

A second Sindbis virus-based expression system SinRep/LacZ is obtained from Invitrogen (Carlsbad, Calif.). This vector encodes the packaging signal, a nonstructural polyprotein nsp1-4 for replicating the RNA transcript, the promoter for subgenomic transcription, and the bacterial β-galactosidase LacZ gene. DH-BB, a helper DNA template that contains the structural genes (capsid, E3, E2, 6K, and E1) required for packaging the virus was also obtained from Invitrogen.

To construct a replication incompetent, non-cytopathic Sindbis viral vector containing genes for sc8H9 (Fv)-PE38, the Sindbis viral vector SinRep/2PSG is used which contains a secondary subgenomic promoter that is responsive to the Sindbis replicase. Two DNA oligonucleotide primers (SEQ ID NO: 72) (sequence 5′CGCGTAAAGCATCTCTACGGTGGTCCTAATAGTGCATG-3′ and its complementary strand (SEQ ID NO: 73) 5′-CACTATTAGGACCACCGTCGAGATGCTTTA-3′) containing the subgenomic promoter sequence is annealed and ligated into the MluI and SphI sites of the SinRep plasmid. This vector producing the native Sindbis virus alone can be used for infection of SS erythroblasts. Alternatively, the sc8H9(Fv)-PE38 is subcloned into the MluI and the StuI sites of SinRep/2PSG, to produce the Sin-Rep/sc8H9(Fv)-PE38 and this vector producing the structural elements of the Sindbis virus and the PE38-MoAb tumor toxin are replicated.

A third plasmid—SINRep/lacZ contains a SP6 promoter, a 7-kb fragment encoding the SIN RNA replicase, and a subgenomic promoter that is bound by the RNA replicase to synthesize large quantity of subgenomic RNA. The LacZ gene is located in the 5′ region of the subgenomic promoter. The plasmid pRep-LacZ is constructed by inserting an Sph\ fragment containing the CMV immediate early promoter/enhancer into the Sph\ site of SINRep-LacZ. An additional SpeI site is inserted 5′ of the SP6 promoter together with the CMV promoter/enhancer fragment. Digestion of the plasmid with SpeI generates a DNA fragment that contains only the SIN RNA replicase and sc8H9 (Fv)-PE38 coding sequences. The primers used for generating a PCR fragment containing the CMV promoter/enhancer from pCR3.1 plasmid (Invitrogen, Carlsbad, Calif.) are: 5′ primer, (SEQ ID NO: 74) 5′-ACATCCATCCACTAGTGCCCCCGTTGACATTGATTA-3′ (SpeI site (underlined) added for subsequent linearization) and 3′ primer (SEQ ID NO: 75), 5′-ACATG-CATGCATGTGAGAGCTCTGCTTATATAGACC-3′. SINrep/LacZ cDNA is placed under the control of the HRE promoter. The cloning strategy is designed to place the previously mapped transcription start site of the HRE promoter close to the 5′ end of the replicon. The promoter PCR HRE product is digested with BspEI and XhoI, and the product is then cloned into NgoMI- and XhoI-digested pSINrep/LacZ by placing a XhoI site at the 5′ terminus of the cDNA of pSINrep/LacZ (FIG. 3).

P987ySINrep19ylacZ vector contains the SINrep19-ylacZ cDNA, a pSinRep5 derivative flanked by the Rous sarcoma virus-long terminal repeat promoter replacing the SP6 promoter positioned upstream of the nonstructural proteins and simian virus polyadenylation signals. Additional point mutation P726S deletes the cytopathic phenotype of the nonstructural protein 2 (nsp2) subunit 18. The tumor toxin genes are cloned into p987SinRep96 via XbaI, Bsp120, I XbaI and StuI.

PE38 is the truncated form of pseudomonas exotoxin A and psc8H9 is the expression vector for sc8H9 (Fv)-PE38 which encodes the PE38 fused to single or double chain tumor specific Fv specific for adenocarcinomas (FIG. 1 of Onda et al., supra (2004)). DNA fragments encoding sc8H9 (Fv)-PE38 are isolated by digesting psc8H9 (Fv)-PE38 with NdeI and EcoRI restriction enzymes. These isolated DNA fragments are further cloned into the corresponding XbaI and PmeI sites of the SINrep5 vector to generate SINrep-sc8H9 (Fv)-PE38. The accuracy of these constructs is confirmed by DNA sequencing.

### In Vitro Transcription and Transfection for Sindbis Viral Vector Production

To generate the Sindbis viral vectors, the vector plasmids are first transcribed in vitro to generate Sindbis viral vector RNA. The RNA is then transfected into cells, where it is translated, replicated, and packaged into viral particles, which are used to infect tumor cells. Plasmids for the in vitro transcription of Sindbis viral RNAs (SinRep/LacZ, Sin-Rep/sc8H9(Fv)-PE38, and DH-BB) are prepared with the Qiagen plasmid kit (Valencia, Calif.). The helper DNA template DH-BB and a replicon plasmid (SinRep/LacZ or SinRep/IL12) are digested with XhoI to linearize the templates. Digested plasmids are purified by phenol-chloroform extraction and ethanol precipitation. In vitro transcription reactions are carried out using the mMESSAGE mMACHINE™ high yield capped RNA transcription kit (SP6 version; Ambion, Inc., Austin, Tex.) to produce capped mRNA transcripts. The quality of the transcribed RNA is checked on 1% agarose gels.

Both DH-BB and SinRep/LacZ or SinRep/sc8H9(Fv)-PE38 RNAs (20 μL each of the in vitro transcription reaction mix) are electroporated into BHK cells. Electroporated cells are transferred into 10 mL of MEM containing 5% FBS and incubated at 37° C. for 12 hours. Adherent BHK cells are then washed with phosphate-buffered saline (PBS) and incubated in 10 mL of Opti-MEM I medium (Invitrogen) without FBS. After 24 hours, culture supernatants are collected and aliquots (3-4 mL) are stored at −80° C.

### Virus Quantification

To quantify the number of viral vector particles in the collected culture supernatants (above), serial dilutions (300 μL each) of an aliquot containing SinRep/LacZ or SinRep/sc8H9 (Fv)-PE38 vectors were added to 2×10⁵ BHK cells in 12-well plates. After incubating for 1 hour at room temperature, the cells were washed with PBS and incubated with 2 mL of MEM at 37° C. for 24 hours. SinRep/LacZ or SinRep/sc8H9(Fv)—PE38 infection is determined by fixing the cells in PBS containing 0.5% glutaraldehyde at room temperature for 20 minutes, washing them three times with PBS, and then staining them with PBS containing 1 mg/mL X-gal (5-bromo-4-chloro-3-indolyl-_-D-galactopyranoside; Fisher Scientific, Pittsburgh, Pa.), 5 mM potassium ferricyanide, 5 mM potassium ferrocyanide, and 1 mM MgSO₄ at 37° C. for 3 hours. After staining with the X-Gal solution, cells that expressed LacZ stained blue. Blue-stained cells were counted and viral titers were estimated by determining the number of LacZ colony-forming units (CFU) per mL of aliquot.

### Transfection and Effects on Transduced Cells

The RNA or DNA vectors are transfected into cells either by lipofection or electroporation. These RNA molecules function as mRNA. The subgenomic RNA synthesized in the transfected cells is translated into the heterologous protein.

### Animal Models and In Vivo Transfection

All animal experiments are performed in accordance with institutional guidelines. All experiments used 6- to 8-week-old SCID mice (C.B-17-SCID and C.B-17-SCID beige mice), which are obtained from Taconic (Germantown, N.Y.). Male and female mice were used for the experiments. BHK cells (5×10⁶) are injected subcutaneously into the right flank of the abdomen of C.B-17-SCID or C.B-17-SCID beige mice. After 10 days, when the BHK tumors have reached a size of at least 1 cm2, the mice are randomly assigned to one of three groups: control (n=5), SinRep/LacZ (n=5) or SinRep/IL12 (n=5). Each mouse in the experimental groups receives a single daily intraperitoneal injection of 0.5 mL of Opti-MEM I containing 10⁷-10⁸ CFU of SinRep/LacZ or Sin-Rep/IL12 viral vector particles. Three control mice receive an injection of 0.5 mL of PBS, and two are left untreated. The day of first treatment is designated day 1. The size of BHK tumors was measured daily and calculated by the formula (π/6)×(length, cm)×(width, cm)². Control mice are followed for 12 days, and treated mice were followed for 5 weeks.

For human tumor models, LS174T, HT29, and CFPAC cells (initial injection of 4×10⁶ cells) are grown as subcutaneous tumors in C.B-17-SCID mice for 4 weeks to allow the tumors to reach a substantial size before treatment was begun. Tumor bearing C.B-17-SCID mice are randomly assigned to control or experimental groups, and they receive daily intraperitoneal treatments of PBS or 0.5 mL SinRep/LacZ containing 10⁷-10⁸ CFU of viral vector particles. Experimental groups are treated for 6-7 weeks. Tumor sizes [(length, cm)×(width, cm)×(height, cm)] are recorded daily. There were four mice per group for experiments with LS174T and CFPAC tumors and five mice per group for experiments with HT29 tumors.

### Example 5

### Preparation of siRNA Duplexes

21-Nucleotide RNAs complementary to target genes as listed in Table 4 are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). A typical 0.2 pmol-scale RNA synthesis provides about 1 milligram of RNA, which is sufficient for 1000 transfection experiments using a 24-well tissue culture plate format.

Transfection of siRNA Duplexes

A single transfection of siRNA duplex is carried out using OLIGOFECTAMINE Reagent (Invitrogen) with assay for silencing 2 days after transfection. Transfection efficiencies are typically around 90-95%. No silencing is observed in the absence of transfection reagent. Oligofectamine has the advantage of being non-toxic to cells and the medium does not to be changed after transfection. siRNA transfection is also possible by using TransIT-TKO: small interfering RNA (siRNA) Transfection Reagent, which is provided by Minis. Transit-TKO reagent is more difficult to handle than OLIGOFECTAMINE, because concentrations required for effective transfection also cause cytotoxic effects. Typical side effects of Transit-TKO siRNA transfection are morphologic changes such as formation of extended lamellipodia as well as oval-shaped nuclei, and which appear about 2 days after transfection. These effects are observed using between 4.0 and 4.5 μl of Transit-TKO reagent. Two other siRNA transfection reagent were recently introduced by Polyplus-transfection SAS, termed jetSI, and by Upstate, termed siIMPORTER.

For one well of a 24-well plate 0.84 μg siRNA duplex (60 pmole in 3 annealing buffer) is used. Mix 3 μl of 20 μM siRNA duplex with 50 μl of Opti-MEM. In another tube, mix 3 μl of OLIGOFECTAMINE Reagent (or 3 to 3.5 μl Transit TKO) with 12 μl of Opti-MEM, incubate 7 to 10 min at room temperature. Combine the solutions and gently mix by inversion. Do not vortex. Incubate another 20 to 25 min at room temperature; the solution turns turbid. Then add 32 μl of fresh Opti-MEM to obtain a final solution volume of 100 μl. (The addition of 32 μl Opti-MEM is optional and serves only to adjust the total volume of cell culture medium to 600 μl after transfection.) Add the 100 μl of siRNA-OLIGOFECTAMINE to cultured cells (40 to 50% confluent). The cells are seeded the previous day in 24-well plates using 500 μl of DMEM tissue culture medium supplemented with 10% FBS but without antibiotics.

Transfection of 0.84 μg single-stranded sense siRNA has no effect and 0.84 μg antisense siRNA has a weak silencing effect when compared to 0.84 μg of duplex siRNAs. However, when the siRNA concentrations are reduced 100-fold no antisense effect is apparent while the siRNA duplex is still efficiently silencing. On this note, it is often possible to reduce the siRNA duplex concentration in order to save precious RNA.

The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful silencing. To control for transfection, we recommend to target laminin A/C and to determine the fraction of laminin A/C knockdown cells by immunofluorescence. Alternatively, a feeling for transfection efficiency may be developed by transfection of a CMV-driven EGFP-expression plasmid (e.g. from Clontech) using Lipofectamine 2000 (Invitrogen). The transfection efficiency is then assessed by phase contrast and fluorescence microscopy the next day.

### Example 6

Videomicroscopy and morphometric analysis of 4T1 mammary carcinomas implanted in the mouse dorsal skin-fold window chamber was used to determine the selectivity of SS erythrocytes for tumor microvasculature and deposition in tumor parenchyma. SS erythrocytes from patients with homozygous SS sickle cell disease and healthy normal donors were labelled with carbocyanine and infused intravenously into tumor bearing mice.

### Methods

Collection and preparation of human RBCs: Fresh blood samples from patients homozygous for hemoglobin S and from normal controls were collected into citrate tubes. RBCs were allowed to separate from the buffy coat containing leukocytes and platelet-rich plasma by gravity at 4° C. for at least 2 h. Plasma and buffy coat were removed by aspiration and RBCs were washed four times in sterile PBS With 1.26 mM Ca²⁺, 0.9 mM Mg²⁺ (pH 7.4). Packed RBCs were analyzed for leukocyte and platelet contamination using an Automated Hematology Analyzer K-1000 (Sysmex, Japan). SS erythroblasts will be generated by in vitro culture of isolated CD34⁺ progenitor cells from peripheral blood-derived mononuclear cells (PBMCs) as previously described (Panzenbock B. et al. Growth and differentiation human stem cell factor/erythropoietin-dependent erythroid progenitor cells in vitro. Blood 92:3658-3668 (1998); Arcasoy M O, Jiang X. Co-operative signaling mechanisms required for erythroid precursor expansion in response to erythropoietin and stem cell factor. Br. J. Haematol. 130:121-129, (2005)).
Treatment of human RBCs: Dil (Molecular Probes Inc., Eugene, Oreg.) dye was used to fluorescently label packed RBCs (150 μl) for in vivo studies as described previously (Unthank J L et al., Microvasc. Res. 45:193-210 (1993)). Such dye has no effect on RBC suspension viscosity nor on the RBC lifetime in the circulation. Cell morphology was checked by microscopy. SS RBCs were treated at 37° C. with 20 nM epinephrine (Sigma) for 1 min. Cells were then washed three times with 5 ml PBS with Ca²⁺ and Mg²⁺. Normal RBCs were similarly sham- or epinephrine-treated.
Mice: All animal experiments were carried out in accordance with protocols approved by the Duke University Animal Care and Use Committee. We used female athymic homozygous nude mice (nu-/nu-), obtained from Charles River Laboratories (Wilmington, Mass.), between 8-12 weeks of age. All infusions were performed using the dorsal tail vein.
Window chamber surgery and murine mammary carcinoma implantation: This procedure was performed as previously described (Algire G H and Legallais F Y. J. Natl. Cancer Inst. 10:225-53 (1949); Kalambur V S et al. Am. J. Hematol. 77:117-125. (2004). General anesthesia was achieved by intraperitoneal injection of 100 mg/kg of ketamine (Abbott Laboratory, Chicago, Ill.) and 10 mg/kg of xylazine (Bayer, Shawnee Mission, Kans.). A window chamber consisting of a double-sided titanium frame was surgically implanted into the dorsal skin fold under sterile conditions with aseptic technique using a laminar flow hood. (Dewhirst M W et al., Dis. Markers 18:293-311 (2002). Surgery involved carefully removing the epidermal and dermal layers of one side of a dorsal skin fold, exposing the blood vessels of the subcutaneous tissue adjacent to the striated muscles of the opposing skin fold. The two sides of the chamber were secured to the skin using stainless steel screws and sutures, followed by injection of 10⁷ 4T1 murine mammary carcinoma cells into the skin. A glass window was placed in the chamber to cover the exposed tissue and secured with a snap ring. Subsequently, animals were kept at 32-34° C. until in vivo studies were performed 8-9 days post-surgery.
Hyperspectral imaging of hemoglobin saturation: In vivo experiments were conducted under a protocol approved by the Duke University Institutional Animal Care and Use Committee. A titanium window chamber was surgically implanted under anesthesia (ketamine 100 mg/kg IP and xylazine 10 mg/kg IP) on the back of athymic nude mice (nu/nu, NCI, Frederick, Md.). A window chamber tumor was established during chamber implantation by injecting 10 μL of a single cell suspension (5×10³ cells) of 4T1 mouse mammary tumor cells into the dorsal skin flap prior to placing a 12 mm diameter #2 round glass coverslip (Erie Scientific, Portsmouth, N.H.) over the exposed skin. The tumor cells constitutively expressed DsRed under the control of the cytomegalovirus promoter, and expressed green fluorescent protein in hypoxic conditions under control of the hypoxia regulatory element (Sorg B S et al., J Biomed Optics 10: article 044004 (2005). For imaging, animals were anesthetized with ketamine (100 mg/kg IP) and xylazine (10 mg/kg IP) and placed on a heating pad attached to the microscope stage. Imaging of tumor microvessel hemoglobin saturation was performed as described previously (37b). Briefly, a Zeiss Axioskop 2 microscope (Carl Zeiss, Inc., Thornwood, N.Y.) served as the imaging platform. Images were acquired with a CCD camera (DVC Company, Austin, Tex.), and bandlimited optical filtering for hyperspectral imaging was accomplished with a C-mounted liquid crystal tunable filter (CRI, Inc., Woburn, Mass.). Image processing was performed using Matlab software (The Mathworks, Inc., Natick, Mass.).
RBC infusions and intravital microscopy: Anesthetized animals implanted with window chambers were infused with a 300 μl bolus (Hct 50% in PBS with Ca²⁺ and Mg²⁺) of a given washed labeled RBC or erythroblast sample to detect human RBC adhesion to tumor endothelium and adjacent normal blood vessels. Animals were placed on the stage of an Axoplan microscope (Carl Zeiss, Thornwood, N.Y.), and temperature was maintained at 37° C. using a thermostatically controlled heating pad. Blood flow dynamics were observed in both tumor neovasculature and subdermal vessels visualized for at least 30 minutes using two different objectives, 20× (high magnification) and 5× (low magnification) (Zeiss). Microcirculatory events and cell adhesion were simultaneous recorded using a video-recording setup consisting of a Trinitron Color video monitor (model PVM-1353 MD, Sony) and JVC video cassette recorder (model BR-S3784, VCR King, Durham, N.C.) connected to a digital video camera C2400 (Hamamatsu Photonics K.K., Japan). Blood vessels were viewed under fluorescence ε-illumination using a 100-W mercury arc lamp and 5× and 20× magnifications for measurement of red cell flux and adhesion.
Histology: Animals were sacrificed 30 minutes or 24 hours post-injection of fluorescently labeled RBCs. Tumor, spleen, lung and kidney were collected and snap frozen. Sections of 40 μm were cut, mounted on slides and examined via inverted fluorescence microscopy (Zeiss). Three random fields were imaged for each organ examined, and fluorescence intensity for each field captured was quantified using Adobe Photoshop CS2 software (Adobe Systems Incorporated, San Jose, Calif.). The values were averaged for the three fields to represent the mean fluorescence intensity. The mean fluorescence values were averaged among groups of animals (n=3) for statistical analysis, using paired t-test (Zennadi R et al., Blood 2007 in press).
Statistical analysis: Data were compared using parametric analyses (GraphPad Prism 4 Software, San Diego, Calif.), including repeated and non-repeated measures of analysis of variance (ANOVA). One-way and two-way ANOVA analyses were followed by Bonferroni corrections for multiple comparisons (multiplying the p value by the number of comparisons). A p<0.05 was considered significant.

### Results

### Hyperspectral Imaging of 4T1 Tumor in Nu/Nu Mice

SS cells are known to display impaired deformability and increased adhesion under hypoxemic conditions. We therefore first analyzed the hemoglobin saturation and oxygen transport of the 4T1 carcinoma in the dorsal skin window with hyperspectral mapping and imaging from day 4 to day 8 after tumor implantation as described in Methods. These studies established that from day 6 onward the tumor and blood vessels permeating the tumor exhibited diffuse hypoxia. Hence, SS cell infusion studies were carried out on day 8 after tumor implantation.

### Human RBC Clearance in Nude Mice.

Our previous study validated the use of nude mice for SS infusion studies since the half life of SS and normal cells in this model is ˜12 hours and >85% of SS cells and normal cells were circulating at 20-30 minutes post-infusion. Therefore, our model system in which infused RBCs were studied up to 30 minutes after infusion provided an sufficient window to evaluate the deposition of infused SS cells in the 4T1 tumors.

Previously, we have shown that dorsal skin-fold window chamber implantation did not induce inflammation and leukocyte adhesion to endothelium (Zennadi et al. supra (2007)). In our studies, the quantity of human RBCs did not exceed 10% of the total circulating RBCs, assuming that the mouse blood volume is 1.5 ml, thereby minimizing any possible rheological effects attributable to increased hematocrit. Human RBCs in these small concentrations has been shown to have no adverse effects on vascular regulatory mechanisms or O₂ delivery.

### SS RBCs but not Normal RBCs Deposit in Tumor Vasculature

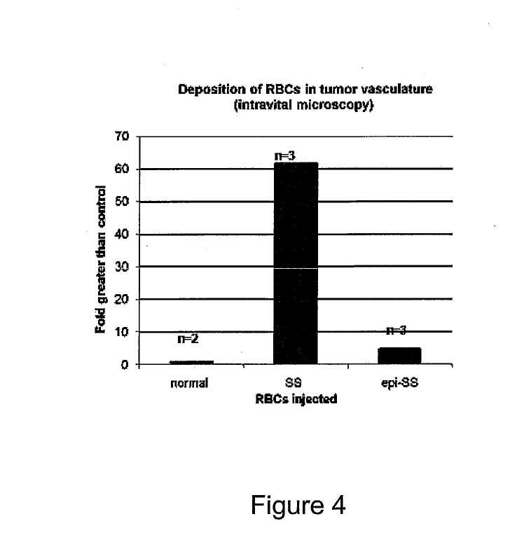

Infusion of SS RBCs into tumor bearing mice resulted in rapid appearance of the fluorescent RBCs in the tumor microvessels. Within 2-5 minutes after infusion of sickle cell adhesion to tumor endothelium was evident progressing to occlusion of a large percentage of blood vessels over a 30 minute period. SS RBC velocity was markedly reduced in multiple areas of the tumor vessels associated with SS RBC stasis in occluded or partially occluded regions. At the same time, SS RBCs showed minimal adhesion to normal subdermal vascular endothelium and adhered only occasionally to small postcapillary venules not larger than 25 μm in diameter with no evident cell stasis or vaso-occlusion. In contrast, normal RBCs showed almost no adhesion to neovascular vessels or adjacent subepidermal vessels. Morphometric analysis showed a 64 fold increase in the deposition of SS cells in the tumor vasculature on intravital microscopy compared to normal RBCs (FIG. 4).

### SS RBCs but not Normal RBCs Deposit in Tumor Parenchyma

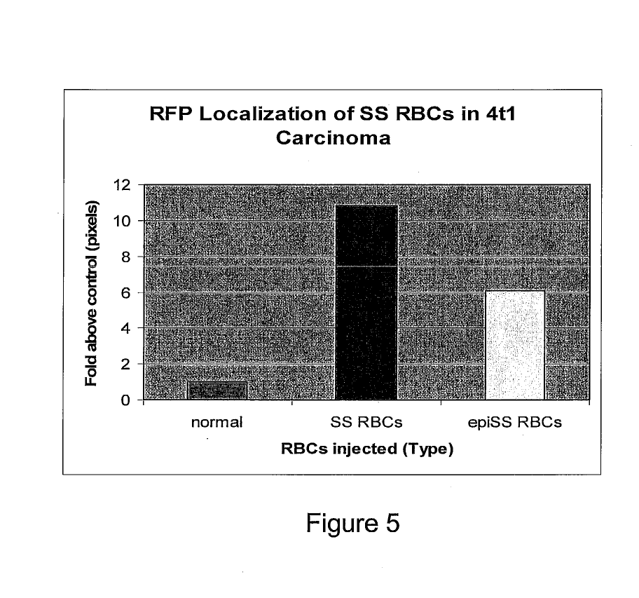

Tumor sections from mice given SS RBCs showed diffuse deposition of rhodamine throughout the tumor parenchyma whereas rhodamine stained normal RBCs showed only minimal deposits binding to tumor tissue. Morphometric comparison of the tumor deposition of rhodamine showed an 11 fold greater uptake by SS RBCs compared to normal RBCs (FIG. 5).

### Epinephrine-Treated SS RBCs Deposit in Tumor Vasculature and Parenchyma

Infusion of epinephrine-treated SS RBCs into tumor bearing mice resulted in diffuse deposition and occlusion of tumor neovessels with focal deposition in adjacent normal vessels. Morphometric analysis showed 4 and 5 fold greater deposits of epinephrine-treated RBCs in tumor vasculature and parenchyma respectively compared to normal RBCs (FIGS. 4 & 5). However, deposition of SS RBCs in tumor vasculature and parenchyma exceeded that of epinephrine-treated SS RBCs by 10 and 2 fold respectively.

### Discussion

The major observation in our study is that SS RBCs in their native state bind and occlude tumor blood vessels and infiltrate the parenchyma of a murine breast carcinoma model. SS RBC uptake in tumor blood vessels is rapid (within 30 minutes after infusion), diffuse and selective for tumor vasculature sparing adjacent normal subdermal blood vessels. Adhesion and vasoocclusion of tumor vessels was seen only with SS but not normal blood vessels and was associated with diffuse uptake of SS cells in tumor parenchyma. Indeed, morphometric determinations showed that SS deposits in tumor neovasculture and parenchyma exceeded those of normal RBCs by 64 and 12 fold respectively. The binding of SS RBCs to murine tumor vasculature could not be attributed to non-specific vascular binding since there was minimal deposition of SS RBCs in spleen, lung and kidney and normal human RBCs failed to localize in the tumor vasculature. Thus, it appears that mature SS RBCs selectively home to the tumor microcirculation leading to diffuse vaso-occlusion of tumor blood, vessels and widespread parenchymal deposition.

The 4T1 mammary carcinoma employed herein is representative of tumor neovasculature in mammary carcinomas displaying the classic hallmarks of vessel tortuosity, density and intermittent flow (Dewhirst M W et al., Radiat Res 130:171-82 (1992); Dewhirst, M W et al., Radiat Res 132: 61-8 (1992); Jain, R K et al., Nature Reviews Cancer 2: 266-276 (2002)). Hyperspectral imaging of this tumor in our laboratory with probes monitoring oxygen levels in the tumor showed that by day 8 after implantation (when the present studies were carried out) hemoglobin saturation was well below 20%. The tumor model therefore permits an examination of the behavior of SS and normal RBCs in an intact hypoxemic carcinoma in the presence of normal blood components, physiologic flow and shear stresses.

We believed that observations made during the first 30 min following infusion of xenogeneic RBCs into nu-/nu- mice would be informative since our previous studies showed that the half life of both SS cells and normal cells in nu/nu mice was ˜12 hours and that >85% of both cell types were still circulating at 20-30 minutes post-infusion consistent with earlier reports in similar models (Ishihara C et al., J. Vet Med. Sci. 1994; 56: 1149-1154 (1994); Butcher G A et al., Exp. Parsitology 77:257-260 (1993)). Minimal amounts of murine immunoglobulin were bound to circulating human normal RBCs and SS RBCs 40 min post-infusion compared to non-infused cells suggesting that naturally occurring mouse antibodies directed to human AB or galactosyl 1,3-galactose antigens expressed on human RBCs were not operative in this system (Rees M A Xenotransplantation 2005; 12:13-9 (2005)).

We observed significant binding and vasoocclusion of SS RBCs, but not normal RBCs, selectively in tumor blood vessels sparing adjacent normal vessels. The selectivity of SS cells for tumors may be explained in part by their impaired mechanical deformability and increased sickling as a consequence of increased hemoglobin polymerization in the relatively deoxygenated tumor milieu (Evans E et al., J Clin Invest. 73:477-88 (1984); Dong C et al., Biophys J. 63:774-83 (1992); Nash G B et al., Blood. 67:110-8 (1986); Itoh T et al., Blood. 85:2245-53 (1995); Kaul D K et al., Blood 77:1353-61 (1991)).

Hypoxia-induced sickling and rigidity of SS RBCs reduced SS cell velocity and increased endothelial contact time in the tumor microcirculation leading to diffuse adhesion the lumenal surface. Widespread adhesion of SS but not normal RBCs to the lumenal surface may also be explained by display of multiple adhesion receptors ICAM-4, BCAM/lu, α4β1 and CD36 only on SS cells whose cognate endothelial counterreceptors αvβ3 integrin, laminin α5, VCAM-1 and membrane-bound intermediary protein thrombospondin respectively are overexpressed on tumor microvasculature.

Our previous studies have suggested a pathological role for SS RBCs ICAM-4 (LW)—the counterreceptor for αvβ3 integrin (Zennadi R et al., Blood 2004; 104:3774-3781 (2004))—in mediating not only SS RBC adhesion to normal post capillary venulues but vaso-occlusive events as well. LW-mediated SS RBC adhesion via interactions with αvβ3 integrin overexpressed on tumor endothelium is therefore likely to play a significant role in the binding of SS cells to the tumor neovasculature. The ligand for the BCAM/lu receptor on SS RBCs is laminin α5 which is also overexpressed in tumor endothelium, in tumor mosaic blood vessels and on breast and lung carcinoma cells (TaninT et al., Exp Cell Res. 248:115-21 (1999); Kikkawa Y et al., J Biol. Chem. 273:15854-9 (1998); Zen Q et al., J Biol. Chem. 274:728-34 (1999); Kubota Y et al., J. Cell Biol. 107:1589-98 (1998)).

SS reticulocytes express α4β1 integrin, which binds tumor endothelial VCAM-1 and fibronectin both of which are expressed in murine tumor endothelium (Ruoslahti E. Biochem Soc Trans. 32(Pt3):397-402 (2004); Jin H and Varner J Br J Cancer 90:561-5 (2004); Kim S et al. Am J Pathol 156:1345-62 (2000)). Thus, up-regulation of these adhesion systems on SS RBCs and tumor endothelial cells coupled with hypoxia-induced increase in mechanical rigidity, reduced blood velocity and increased contact time with the vessel wall led to the selective uptake of SS RBCs in the tumor microcirculation.

Epinephrine-treated RBCs which are known to display upregulated BCAM/Lu and ICAM-4 also localized to the tumor neovasculature but to a lesser degree than untreated SS cells. This may be explained by their increased binding to normal blood vessels compared to untreated SS RBCs. Although adhesion of epinephrine-treated SS RBCs to endothelium in vivo appears to play a key role in the induction of sickle cell crisis precipitated by stress, in the present system untreated SS RBCs bind more effectively to tumor vessels and display more vasoocclusion without significant binding to normal vessels than epinephrine treated SS cells. Hence, epinephrine treatment of SS cells is not requisite for sickle cell adhesion and vasoocclusion in the tumor vasculature, SS RBC adhesion and vaso-occlusion in the tumor vasculature were as striking in our studies as adhesion and vaso-occlusion observed in murine models of sickle cell disease by upregulating NFkB or using proinflammatory cytokines such as platelet activating factor (PAF) and tumor necrosis factor-α (TNF-α), which primarily affect endothelial cells, but also activate leukocytes and platelets (Kaul et al., D K Blood 2000; 95:368-374 (2000) Zhang C et al., Arterioscler Thromb Vasc Biol. 26:475-80 (2006)).

Proinflammatory cytokines were not administered in this study however hypoxia-induced production of TNFα and VEGF by tumors may upregulate tumor vascular adhesion molecules and laminin α5. In this sense, the tumor neovasculature may mimic the sickle endothelium functionally which likewise displays upregulated adhesion molecules due to multiple local oxygen-reperfusion defects (Kaul D K & Hebbel R P. J Clin Invest. 106:411-20 (2000); Dewhirst M Cancer Res. 67:854-5 (2007); Cardenas-Navia L I et al., Cancer Res. 64: 6010-7 (2004)). In humans, the frequency of symptomatic vaso-occlusive events is low when the percentage of SS RBCs is below 20-30%. However, we observed vaso-occlusion in the tumors in our experiments with SS RBCs, even though the percentage of infused SS RBCs never exceeded 10% of the total circulating RBCs. These results suggest that a small percentage of SS RBCs may be sufficient to initiate selective vaso-occlusion in tumors in vivo.

The heterogeneity in expression of tumor adhesion molecules due to regional predominance of pro- (TNFα and VEGF) and anti- (TGFβ and basic FGF) adhesion molecules and spatially diversified blood perfusion in tumors has made therapeutic targeting of tumor vessels challenging and difficult. SS RBCs with their multiple activated adhesion systems and impaired deformability showed selective uptake in tumor neovasculature where they induced vaso-occlusion and diffuse tumor parenchymal deposits of rhodamine-labelled SS cells including hypoxemic sectors considered to be chemo- and radioresistant. Thus, SS RBCs and their nucleated progenitors may possess a combination of mechanical and adhesive properties suitable for targeting primary and occult metastatic tumors with hypoxia-sensitive chemotherapy, oncolytic viruses and toxins.

### Example 7

Methods for SS cell encapsulation, optionally incorporating ferrous molecules are described below (G. L. Dale, et al., Biochem. Med. 18, 220 (1977); DeLoach JR & Sprandel U (eds.), “Red Blood Cells as Carriers for Drugs.” Karger-Verlag, Basel, Switzerland, (1985): Zimmermann U. et al., Biochim. Biophys. Acta 436: 460 (1976); DeLoach & Ihler G. Biochim Biophys Acta 496: 136 (1977)) which are herein incorporated by reference.

### Materials

Buffer I (isosmotic): 150 mM NaCl, 5 mM K₂HPO₄/KH₂PO₄, pH 7.4.
Buffer II (isosmotic): like buffer 1, in addition 10 mM glucose, 5 mM adenosine, 1 mM MgCl₂. Buffer III (hyposmotic): 5 mM K₂HPO₄/KH₂PO₄, pH 7.4.

### Preparation of Erythrocyte Ghosts

Erythrocyte ghosts are prepared by a hypotonic dialysis procedure with best results obtained after standardization of the following parameters. Washed red blood cells are placed into dialysis tubing. Then a solution of buffer I and (optionally) the ferrofluids to be entrapped (25% ferrofluids in buffer I) is added. The hematocrit is 75% (three volume units of red blood cells and one volume unit of buffer I-ferrofluids solution). The erythrocytes are then dialyzed against hyposmotic buffer III for 75 min at 4° C. After entrapment, cells are resealed by dialysis against isosmotic buffer II for 60 min at 37° C. After preparation, the resealed cells are washed again four times by centrifugation in isosmotic buffer II (530 g, 20 min at 20° C.).

### Ferrofluids (Optional)

Ferrofluids are permanent, colloidal suspensions of magnetite (Fe₃O₄), produced and delivered by Ferrofluidics Corporation in Nashua, N.H. The only kind of ferrofluids used in these experiments are type BIO-I (catalog no. EMG 1111), a water-based ferrofluid preparation that contains no organic or inorganic detergents. The properties of BIO-I are: magnetic saturation, 200 G; density, 1.18 g/ml; viscosity, 1-10 cP, 27° C.; vapor pressure, 100° C., 760 mmHg; initial susceptibility, 0.6; magnetite particles, 25 vol % of the suspension; particle size, 8-20 nm, average 18 nm.

An additional method for preparation of sickle cell ghosts is described by Bax et al., Clin Sci. 96: 171-178 (1999)

### Blood Preparation

Forty milliliters of blood are collected from patients with homozygous SS sickle cell anemia and placed into two tubes containing 4 ml of anticoagulant citrate phosphate dextrose or 200 units of heparin. The blood samples are centrifuged for 10 min at 1100 g; the supernatant plasma is removed and kept for later use and the buffy coat is discarded. The erythrocytes are washed twice in cold (4° C.) iso-osmotic PBS, pH 7.4 (2.68 mM/1 KCl, 1.47 mM/1 KH₂PO₄, 136.89 mM/l NaCl, 8.10 nM/1 Na₂HPO₄), and centrifuged for 10 min at 1100 g.

### Carrier Erythrocyte Preparation

Carrier erythrocytes are prepared using a hypo-osmotic dialysis technique. Washed and packed fresh erythrocytes (10.5 ml) are mixed with 4.5 ml of cold PBS. Five milliliters of this cell suspension are placed into each of three dialysis bags (molecular mass cut-off of 12,000 Da, Medicell International Ltd, London, U.K.) sealed at both ends with clips. Each dialysis bag is placed in a container and supported firmly by wedging the dialysis clips against the container side. Dialysis is against 150 ml of hypo-osmotic phosphate buffer, pH7.4 (5 mM/1 KH₂PO₄, 5 mM/1 K₂HPO₄), at 4° C. in a refrigerated incubator with rotation at 6 rev./min. Macromolecule entrapment can be increased by doubling the hypo-osmotic dialysis time to 180 min. We therefore dialyzed the erythrocytes for 90 min to ascertain that cell survival in vivo is not adversely affected by an extended hypo-osmotic dialysis period. The lysed erythrocytes are resealed by transferring the dialysis bags to containers holding 150 ml of PBS supplemented with 5 mM/1 adenosine, 5 mM/l glucose and 5 mM/l MgCl₂, and continuing rotation at 6 rev/min in the incubator at 37° C. for 60 min. Carrier erythrocytes are washed three times in 3 volumes of supplemented PBS with centrifugation at 100 g for 15 min and finally pooled.

### SUMMARY

Therapeutics of cancer face perhaps the most significant problem, which is specificity and targeting of anti-tumor agents into the tumor while sparing normal tissues. Tumor neo-vasculature is a hypoxemic milieu, with a convoluted ultrastructure containing upregulated expression of multiple activated endothelial adhesion molecules. Erythrocytes from patients with sickle cell anemia (SS RBCs), when exposed to deoxygenating conditions, demonstrate increased deoxy-hemoglobin polymerization, forming rigid, sickled cells that tend to become entrapped in hypoxic tissues. SS RBCs also adhere abnormally to a variety of endothelial ligands, especially when either SS RBCs or endothelial cells have an activated phenotype. Based on this constellation of properties of both SS RBCs and tumor neo-vessels, we hypothesized that SS RBCs could selectively home to the hypoxemic milieu of tumor neo-vasculature and adhere to neo-vessels, which contain multiple cognate counterreceptors for SS RBC adhesion molecules. To demonstrate this, we have infused carbocyanine-labeled SS RBCs into nude mice bearing 8 day old 4T1 mammary carcinoma visible though implanted dorsal skin window chambers and monitored SS RBC behavior via intravital microscopy. Hyperspectral hemoglobin saturation studies of this carcinoma demonstrated significant hypoxemia at day 8 post-implantation. We have discovered that, within 5 minutes after SS RBC infusion, RBCs were diffusely deposited in the tumor neo-vasculature, with minimal adhesion and retention, in adjacent normal subdermal skin blood vessels. SS RBC uptake increased progressively in the tumor vasculature over a 30 minute observation period, promoting vaso-occlusion associated with markedly reduced flow velocity. In contrast, normal RBCs showed no apparent adhesion or deposition in tumor neo-vasculature. Furthermore, while tumor parenchyma showed greater diffuse accumulation of SS RBCs than normal RBCs, SS RBC accumulation in lungs, spleen and kidneys was minimal. These data suggest that due to hypoxia-inducing sickling, and activation and/or up-regulation of expression of endothelial receptors, SS RBCs selectively home to tumor neo-vasculature, resulting in vaso-occlusion, in addition to infiltration of tumor parenchyma. Based on the unique physical and adhesive features of SS RBCs and properties of the tumor vascular endothelium, we hypothesize that (i) the known adhesion receptors overexpressed or activated on SS RBCs play a role in the binding of SS RBCs to the tumor neo-vasculature; (ii) Since SS erythroblasts bear most of the critical adhesion receptors of circulating SS RBCs, SS erythroblasts can home selectively to tumor neo-vasculature, (iii) Because the presence of a nucleus confers erythrocyte progenitors with the capacity for transfection with a wide variety of oncolytic/angiolytic viruses and drugs, SS erythroblasts will be able to deliver oncolytic viruses, chemotherapy or anti-angiogenic agents into the tumor neo-vasculature and induce a tumoricidal response.

All the references, patents and patent applications cited above in this patent application and their references are incorporated by reference in entirety, whether specifically incorporated or not. In addition, the following co-pending patent applications and their references are incorporated by reference in their entirety:

| Inventor | Ser. No. | Filing Date | Title |
|---|---|---|---|
| Terman, D. S. | 60/842,213 | Sept. 5, 2006 | Sickled erythrocytes & Nucleated Precursors for |
| | | | Targeted Delivery of Oncolytic Toxins, Viruses, |
| | | | hemolysins and chemotherapy |
| Terman, D. S. | 60/819,551 | Jul. 8, 2006 | Sickled erythrocytes & Nucleated Precursors for |
| | | | Targeted Delivery of Oncolytic Toxins, Viruses, |
| | | | hemolysins and chemotherapy |
| Terman, D. S. | 60/809,553 | May 30, 2006 | Sickled erythrocytes & Nucleated Precursors for |
| | | | Targeted Delivery of Oncolytic Toxins, Viruses, |
| | | | hemolysins and chemotherapy |
| Terman, D. S. | 60/799514 | May 10, 2006 | Synergy of Superantigens, Cytokines and |
| Bohach, G | | | Chemotherapy in Treatment of Malignant Disease |
| Terman, D. S, | PCTUS05/022638 | Jun. 27, 2005 | Enterotoxin Gene Cluster Superantigens (egc) to Treat |
| Etiene, J., | | | Malignant Disease |
| Vandenesch, F., | | | |
| Lina, G. | | | |
| Bohach, G. | | | |
| Terman, D. S, | 60/583,692 | Jun. 29, 2004 | Enterotoxin Gene Cluster Superantigens (egc) to Treat |
| Etiene, J., | | | Malignant Disease |
| Vandenesch, F., | | | |
| Lina, G. | | | |
| Bohach, G. | | | |
| Terman, D. S. | 60/665,654 | Mar. 23, 2005 | Enterotoxin Gene Cluster Superantigens (egc) to Treat |
| | | | Malignant Disease |
| Terman, D. S, | 60/626,159 | Nov. 6, 2004 | Enterotoxin Gene Cluster Superantigens (egc) to Treat |
| Etiene, J., | | | Malignant Disease |
| Vandenesch, F., | | | |
| Lina, G. | | | |
| Bohach, G. | | | |
| Terman, D. S. | 60/583,692 | Jun. 29, 2004 | Intrathecal and Intrapleural Superantigens to Treat |
| | | | Malignant Disease |
| Terman, D. S. | 60/550,926 | Mar. 5, 2004 | Intrathecal and Intrapleural Superantigens to Treat |
| | | | Malignant Disease |
| Terman, D. S. | 60/539,863 | Jan. 27, 2004 | Intrathecal and Intrapleural Superantigens to Treat |
| | | | Malignant Disease |
| Terman, D. S. | PCT/US03/ | May 8, 2003 | Intrathecal and Intrapleural Superantigens to Treat |
| | 14381 | | Malignant Disease |
| Terman, D. S. | 10/428,817 | May 5, 2003 | Composition and Methods for Treatment of Neoplastic |
| | | | Malignant Disease |
| Terman, D. S. | 60/438,686 | Jan. 9, 2003 | Intrathecal and Intrapleural Superantigens to Treat |
| | | | Malignant Disease |
| Terman, D. S. | 60/415,310 | Oct. 1, 2002 | Intrathecal and Intratumoral Superantigens to Treat |
| | | | Malignant Disease. |
| Terman, D. S. | 60/406,750 | Aug. 29, 2002 | Intrathecal Superantigens to Treat Malignant Fluid |
| | | | Accumulation |
| Terman, D. S. | 60/415,400 | Oct. 2, 2002 | Composition and Methods for Treatment of Neoplastic |
| | | | Diseases |
| Terman, D. S. | 60/406,697 | Aug. 28, 2002 | Compositions and Methods for Treatment of |
| | | | Neoplastic Diseases |
| Terman, D. S. | 60/389,366 | Jun. 15, 2002 | Compositions and Methods for Treatment of |
| | | | Neoplastic Diseases |
| Terman, D. S. | 60/378,988 | May 8, 2002 | Compositions and Methods for Treatment of |
| | | | Neoplastic Diseases |
| Terman, D. S. | 09/870,759 | May 30, 2001 | Compositions and Methods for Treatment of |
| | | | Neoplastic Diseases |
| Terman, D. S. | 09/751,708 | Dec. 28, 2000 | Compositions and Methods for Treatment of |
| | | | Neoplastic Diseases |

Having now fully described this invention, it will be appreciated by those skilled in the art that the same can be performed within a wide range of equivalent parameters, concentrations, and conditions without departing from the spirit and scope of the invention and without undue experimentation.

We claim:

1. A therapeutic mammalian cell transfected with an oncolytic virus wherein said transfected cell induces tumoricidal effects when administered in vivo.

2. The cell of claim 1 which is selected from a group comprising:
a) a sickled erythrocyte
b) a sickled progenitor cell
c) an leukemia cell
d) a carcinoma cell
e) an erythroid progenitor cell
f) a peripheral blood mononuclear cell
g) an erythrocyte
h) an endothelial cell

3. A sickled erythrocyte, sickled nucleated precursor or erythroleukemia cell carrying oncolytic viruses, anti-tumor proteins, plasmids, toxins and chemotherapy, capable of selectively localizing in tumor neovasculature in vivo after parenteral administration.

4. A method of targeting tumoricidal agents to cancer cells in a mammal in vivo comprising administering erythrocytes or nucleated erythroid precursors containing homozygous S or sickle thalassemia hemoglobin capable of localizing selectively in tumor neovasculature.

5. The method of claim 4 where the erythrocyte or nucleated erythrocyte precursor contains hemoglobin selected from a group comprising, hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia.

6. The methods claims 4 and 5 wherein the nucleated erythroid precursor cells are transfected with nucleic acids encoding an oncolytic virus wherein said cells are capable of shedding said virus after said cells are localized in tumor neovasculature.

7. The methods claims 4 and 5 wherein the nucleated SS erythroid precursor cell is transfected with nucleic acid encoding a tumoricidal protein.

8. The tumoricidal protein of claim 7 comprising a group consisting of a superantigen, a pseudomonas exotoxin, a bacterial leukocidin toxin, diptheria toxin, pertussis toxin, a hemolytic toxin, tumor and/or tumor neovasculature specific antibodies or antibody fragments alone or conjugated to said antitumor proteins.

9. The method of claims 4 and 5 wherein the SS cells and nucleated erythroid precursors are loaded with anticancer drugs.

10. A method of treating cancer in a mammal in vivo comprising administering a nucleated erythroleukemic cell, expressing an adhesion molecule which binds to its counterreceptor expressed on tumor microvessels rendering said cell capable of localizing selectively in tumor neovasculature after parenteral administered in vivo.

11. The erythroleukemic cell of claim 10 wherein said cell is rendered resistant to an anticancer drug ex vivo and capable of expelling said drug after said cell is localized in tumor neovasculature.

12. The erythroleukemic cell of claim 10 wherein said cell is infected ex vivo with an oncolytic virus that is released from the cell after said cell is localized in tumor neovasculature.

13. A method of treating cancer wherein the cells of claims 1 and 2 are loaded with oncolytic toxins, antitumor proteins, plasmids, toxins and chemotherapy are exposed to photosensitizers and/or phototherapy ex vivo before parenteral administration to induce:
(a) photohemolysis in vivo after selective localization in tumor neovasculature, and;
(b) shedding of antitumor cell contents into the tumor milieu


SEQUENCE LISTING (75 sequences)

SEQ ID NO: 1 - protein, 231 aa, Staphylococcus sp.
SEKSEEINEKDLRKKSELQGTAGNKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKSINSENMHIDIYLYTS

SEQ ID NO: 2 - protein, 238 aa, Staphylococcus sp.
ESQPDPKPDELHKSSKFTGLMENMKVLYDDNHVSAINVKSIDQFLYFDLIYSIKDTKLGNYDNVRVEFKNKDLADKYKDKYVDVFGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDVKIEVYLTTKK

SEQ ID NO: 3 - protein, 240 aa, Staphylococcus sp.
MNKSRFISCVILIFALILVLFTPNVLAESQPDPTPDELHKASKFTGLMENMKVLYDDHYVSATKVKSVDKFLAHDLIYNISDKKLKNYDKVKTELLNEGLAKKYKDEVVDVYGSNYYVNCYFSSKDNVGKVTGGKTCMYGGITKHEGNHFDNGNLQNVLIRVYENKRNTISFEVQTDKKSVTAQELDIKARNFLINKKNLYEFNSSPYETGYIKFIENNGNTFWYDMMPAPGDKFDQSKY

SEQ ID NO: 4 - protein, 239 aa, Staphylococcus sp.
ESQPDPTPDELHKSSEFTGTMGNMKYLYDDHYVSATKVMSVDKFLAHDLIYNISDKKLKNYDKVKTELLNEDLAKKYKDEVVDVYGSNYYVNCYFSSKDNVGKVTGGKTCMYGGITKHEGNHFDNGNLQNVLIRVYENKRNTISFEVQTDKKSVTAQELDIKARNFLINKKNLYEFNSSPYETGYIKFIENNGNTFWYDMMPAPGDKFDQSKYLMMYNDNKTVDSKSVKIEVHLTTKNG

SEQ ID NO: 5 - protein, 266 aa, Staphylococcus sp.
MYKRLFISRVILIFALILVISTPNVLAESQPDPMPDDLHKSSEFTGTMGNMKYLYDDHYVSATKVKSVDKFLAHDLIYNISDKKLKNYDKVKTELLNEDLAKKYKDEVVDVYGSNYYVNCYFSSKDNVGKVTGGKTCMYGGITKHEGNHFDNGNLQNVLVRVYENKRNTISFEVQTDKKSVTAQELDIKARNFLINKKNLYEFNSSPYETGYIKFIENNGNTFWYDMMPAPGDKFDQSKYLMMYNDNKTVDSKSVKIEVHLTTKNG

SEQ ID NO: 6 - protein, 258 aa, Staphylococcus sp.
MKKFNILIALLFFTSLVISPLNVKANENIDSVKEKELHKKSELSSTALNNMKHSYADKNPIIGENKSTGDQFLENTLLYKKFFTDLINFEDLLINFNSKEMAQHFKSKNVDVYPIRYSINCYGGEIDRTACTYGGVTPHEGNKLKERKKIPINLWINGVQKEVSLDKVQTDKKNVTVQELDAQARRYLQKDLKLYNNDTLGGKIQRGKIEFDSSDGSKVSYDLFDVKGDFPEKQLRIYSDNKTLSTEHLHIDIYLYEK

SEQ ID NO: 7 - protein, 257 aa, Staphylococcus sp.
MKKTAFILLLFIALTLTTSPLVNGSEKSEEINEKDLRKKSELQRNALSNLRQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIDLYLYTT

SEQ ID NO: 8 - protein, 234 aa, Staphylococcus sp.
MNKKLLMNFFIVSPLLLATTATDFTPVPLSSNQIIKTAKASTNDNIKDLLDWYSSGSDTFTNSEVLDNSLGSMRIKNTDGSISLIIFPSPYYSPAFTKGEKVDLNTKRTKKSQHTSEGTYIHFQISGVTNTEKLPTPIELPLKVKVHGKDSPLKYGPKFDKKQLAISTLDFEIRHQLTQIHGLYRSSDKTGGYWKITMNDGSTYQSDLSKKFEYNTEKPPINIDEIKTIEAEIN

SEQ ID NO: 9 - protein, 242 aa, Staphylococcus sp.
MNKIFRVLTVSLFFFTFLIKNNLAYADVGVINLRNFYANYQPEKLQGVSSGNFSTSHQLEYIDGKYTLYSQFHNEYEAKRLKDHKVDIFGISYSGLCNTKYMYGGITLANQNLDKPRNIPINLWVNGKQNTISTDKVSTQKKEVTAQEIDIKLRKYLQNEYNIYGFNKTKKGQEYGYKSKFNSGFNKGKITFHLNNEPSFTYDLFYTGTGQAESFLKIYNDNKTIDAENFHLDVEISYEKTE

SEQ ID NO: 10 - protein, 258 aa, Staphylococcus sp.
MKKLSTVIIILILEIVFHNMNYVNAQPDLKLDELNKVSDKNNKGTMGNVMNLYTSPPVEGRGVINSRQFLSHDLIFPIEYKSYNEVKTELELENTELANNYKDKKVDIFGVPYFYTCIIPKSEPDINQNFGGCCMYGGLTFNSSENERDKLIYVQVTIDNRQSLGFTITTNKNMVTIQELDYKARHWTKEKKLYEFDGSAFESGYIKFTEKNNTSFWFDLFPKKELVPFVPYKFLNIYGDNKVVDSKSIKMEVFLNTH

SEQ ID NO: 11 - protein, 217 aa, Staphylococcus sp.
EDLHDKSELTDLALANAYGQYNHPFIKENIKSDEISGEKDLIFRNQGDSGNDLRVKFATADLAQKFKNKNVDIYGASFYYKCEKISENISECLYGGTTLNSEKLAQERVIGANVWVDGIQKETELIRTNKKNVTLQELDIKIRKILSDKYKIYYKDSEISKGLIEFDMKTPRDYSFDIYDLKGENDYEIDKIYEDNKTLKSDDISHIDVNLYTKKKV

SEQ ID NO: 12 - protein, 242 aa, Staphylococcus sp.
MKKFKYSFILVFILLFNIKDLTYAQGDIGVGNLRNFYTKHDYIDLKGVTDKNLPIANQLEFSTGTNDLISESNNWDEISKFKGKKLDIFGIDYNGPCKSKYMYGGATLSGQYLNSARKIPINLWVNGKHKTISTDKIATNKKLVTAQEIDVKLRRYLQEEYNIYGHNNTGKGKEYGYKSKFYSGFNNGKVLFHLNNEKSFSYDLFYTGDGLPVSFLKIYEDNKIIESEKFHLDVEISYVDSN

SEQ ID NO: 13 - protein, 268 aa, Staphylococcus sp.
MKKTIFILIFSLTLTLLITPLVYSDSKNETIKEKNLHKKSELSSITLNNLRHIYFFNEKGISEKIMTEDQFLDYTLLFKSFFISHSQYNDLLVQFDSKETVNKFKGKQVDLYGSYYGFQCSGGKPNKTACMYGGVTLHENNQLYDTKKIPINLWIDSIRTVVPLDIVKTNKKKVTIQELDLQARYYLHKQYNLYNPSTFDGKIQKGLIVFHTSKEPLVSYDLFNVIGQYPDKLLKIYQDNKIIESENMHIDIYLYTSLIVLISLPLVL

SEQ ID NO: 14 - protein, 242 aa, Staphylococcus sp.
MKKLISILLINIIILGVSNNASAQGDIGIDNLRNFYTKKDFINLKDVKDNDTPIANQLQFSNESYDLISESKDFNKFSNFKGKKLDVFGISYNGQCNTKYIYGGITATNEYLDKPRNIPINIWINGNHKTISTNKVSTNKKFVTAQEIDIKLRRYLQEEYNIYGHNGTKKGEEYGHKSKFYSGFNIGKVTFHLNNNDTFSYDLFYTGDDGLPKSFLKIYEDNKTVESEKFHLDVDISYKETK

SEQ ID NO: 15 - protein, 240 aa, Staphylococcus sp.
MKKRLLFVIVITLFIFSSNHTVLSNGDVGPGNLRNFYTKYEYVNLKNVKDKNSPESHRLEYSYKNDTLYAEFDNEYITSDLKGKNVDVFGISYKYGSNSRTIYGGVTKAENNKLDSPRIIPINLIINGKHQTVTTKSVSTDKKMVTAQEIDVKLRKYLQDEFNIYGHNDTGKGKEYGTSSKFYSGFDKGSVVFHMNDGSNFSYDLFYTGYGLPESFLKIYKDNKTVDSTQFHLDVEISKR

SEQ ID NO: 16 - protein, 239 aa, Staphylococcus sp.
MKRILIIVVLLFCYSQNHIATADVGVLNLRNYYGSYPIEDHQSINPENNHLSHQLVFSMDNSTVTAEFKNVDDVKKFKNHAVDVYGLSYSGYCLKNKYIYGGVTLAGDYLEKSRRIPINLWVNGEHQTISTDKVSTNKKLVTAQEIDTKLRRYLQEEYNIYGFNDTNKGRNYGNKSKFSSGFNAGKILFHLNDGSSFSYDLFDTGTGQAESFLKIYNDNKTVETEKFHLDVEISYKDES

SEQ ID NO: 17 - protein, 263 aa, Staphylococcus sp.
MKNSKVMLNVLLLILNLIAICSVNNAYANEEDPKIESLCKKSSVGPIALHNINDDYINNRRFTTVKSIVSTTEKFLDFDLLFKSINWLDGISAEFKDLKEFSSSAISKEFLGKYVDIYGVYYKAHCHGEHQVDTACTYGGVTPHENNKLSEPKNIGVAVYKDNVNVNVNTFIVTTDKKKVYAQELDIKVRTKLNNAYKLYDRMTSDVQKGYIKFHSHSEHKESFYYDLFYIKGNLPDQYLQIYNDNKTTIDSSDYHIDVYLFT

SEQ ID NO: 18 - protein, 257 aa, Staphylococcus sp.
MKNIKKLMRLFYIAAIIITLLCLINNNYVNAEVDKKDLKKKSDLDSSKLFNLTSYYTDITWQLDESNKISTDQLNNYIILKNIDISVLKTSSLKVEFNSSDLANQFKGKNIDIYGLYFGNKCVGLTEEKTSCLYGGVTIHDGNQLDEEKVIGVNGFKDGVQQEGFVIKTKKAKVTVQELDTKVRFKLENLYKIYNKDTGNIQKGCIFFHSHNHQDQSFYYDLYNVKGSVGAEFFQFYSDNRTVSSSNYHIDVFLYKD

SEQ ID NO: 19 - protein, 132 aa, Staphylococcus sp.
MKLFAFIFICVKSCSLLFMLNGNPKPEQLNKASEFTGLMDNMRYLYDDKHVSETNIKSQEKFLQHDLLFKINGSKILKTEFNNKSLSDKYKNKNVDLFGTNYYNQCYFSLDNMELNDGRLIEKNVYVWRCGL

SEQ ID NO: 20 - protein, 108 aa, Staphylococcus sp.
MYGGVVYENERNSLSFDIPTNKKNITAQEIDYKVRNYLLKHKNLYEFNSSPYETGYIKFIEGSGHSFWYDLMPESGKKFYPTKYLLIYNDNKTVESKSINVEVHLTKK

SEQ ID NO: 21 - protein, 258 aa, Staphylococcus sp.
MSKMKKTAFTLLLFIALTLTTSPLVNGSEKSEEINEKDLRKKSELQGTALGNLKQIYYNEKAKTENKESHDQFLQHTILFKGFFTDHSWYNDLLVDFDSKDIVDKYKGKKVDLYFAYYGYQCAGGTPNKTACMYGGVTLHDNNRLTEEKKEPINLWLDGKQNTVPLETVKTNKKVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 22 - protein, 255 aa, Staphylococcus sp.
MPIWRCNIKKGAIKMNKIFRILTVSLFFFTFLIKNNLAYADVGVINLRNFYANYEPEKLQGVSSGNFSTSHQLEYIDGKYTLYSQFHNEYEAKRLKDHKVDIFGISYSGLCNTKYMGGITLANQNLDKPRNIPINLWVNGKQNTISTDKVSTQKKEVTAQEIDIKLRKYLQNEYNIYGFNKTKKGGEYGYQSKFNSGFNKGKITFHLNNEPSFTYDLFYTGTGGAESFLKIYNDNKTIDAENFHLDVEISYEKTE

SEQ ID NO: 23 - protein, 259 aa, Staphylococcus sp.
MLNKILLLLFSVTFMLLFFSLHSVSAKPDPRPGELNRVSDYKKNKGTMGNVESLYKDKAVIAENVKNTRQFLGHDLIFPIPYSEYKEVKSEFINKKTADKFKDKRLDVFGIPYFYTCLVPKNESREEFIFDGVCIYGGVTMHSTADSISKNIIVPVTVDNKQQFSFTISTNKKTVTVQELDYKVRNWLTNNKKLYEFDGSAYETGYIKFIEQNKDSFWYDLFPKKDLVPFIPYKFVNIYGDNKTIDASSVKIEVHLTTM

SEQ ID NO: 24 - protein, 261 aa, Staphylococcus sp.
MKLFAFIFICVKSCSLLFMLNGNPRPEQLNKASEFSGLMDNMRYLYDDKHVSETNIKAQEKFLQHDLLFKINGSKIDGSKILKTEFNNKSLSDKYKNKNVDLFGTNYYNQCYFSADNMELNDGRLIEKTCMYGGVTEHDGNQIDKNNLTDNSHNILIKVYENERNTLSFDISTNMKNITAQEIDYKVRNYLLKHKNLYEFNSSPYESGYIKFIEGNGHSFWYDMMPESGEKFYPTKYLLIYNDNKTVESKSINVEVHLTKK

SEQ ID NO: 25 - protein, 251 aa, Streptococcus pyrogenes
MENNKKVLKKMVFFVLVTFLGLTISQEVFAQQDPDPSQLHRSSLVKNLQNIYFLYEGDPVTHENVKSVDQLLSHDLIYNVSGPNYDKLKTELKNQEMATLFKDKNVDIYGVEYYHLCYLCENAERSACIYGGVTNHEGNHLEIPKKIVVKVSIDGIQSLSFDIETNKKMVTAQELDYKVRKYLTDNKQLYTNGPSKYETGYIKFIPKNKESFWFDFFPEPEFTQSKYLMIYKDNETLDSNTSQIEVYLTTK

SEQ ID NO: 26 - protein, 398 aa, Streptococcus pyrogenes
MNKKKLGIRLLSLLALGGFVLANPVFADQNFARNEKEAKDSAITFIQKSAAIKAGARSAEDIKLDKVNLGGELSGSNMYVYNISTGGFVIVSGDKRSPEILGYSTSGSFDANGKENIASFMESYVEQIKENKKLDTTYAGTAEIKQPVVKSLLDSKGIHYNQGNPYNLLTPVIEKVKPGEQSFVGQHAATGCVATATAQIMKYHNYPNKGLKDYTYTLSSNNPYFNHPKNLFAAISTRQYNWNNILPTYSGRESNVQKMAISELMADVGISVDMDYGPSSGSAGSSRVQRALKENFGYNQSVHQINRSDFSKQDWEAQIDKELSQNQPVYYQGVGKVGGHAFVIDGADGRNFYHVNWGWGGVSDGFFRLDALNPSALGTGGGAGGFNGYQSAVVGIKP

SEQ ID NO: 27 - protein, 234 aa, Streptococcus pyrogenes
MKKINIIKIVFIITVILISTISPIIKSDSKKDISNVKSDLLYAYTITPYDYKDCRVNFSTTHTLNIDTQKYRGKDYYISSEMSYEASQKFKRDDHVDVFGLFYILNSHTGEYIYGGITPAQNNKVNHKLLGNLFISGESQQNLNNKIILEKDIVTFQEIDFKIRKYLMDNYKIYDATSPYVSGRIEIGTKDGKHEQIDLFDSPNEGTRSDIFAKYKDNRIINMKNFSHFDIYLE

SEQ ID NO: 28 - protein, 260 aa, Staphylococcus sp.
MNKRIRILVVACVVFCAQLLSISVFASSQPDPTPEQLNKSSQFTGVMGNLRCLYDNHFVEGTNVRSTGQLLQHDLIFPIKDLKLKNYDSVKTEFNSKDLAAKYKNKDVDIFGSNYYYNCYYSEGNSCKNAKKTCMYGGVTEHHRNQIEGKFPNITVKVYEDNENILSFDITTNKKQVTVQELDCKTRKILVSRKNLYEFNNSPYETGYIKFIESSGDSFWYDMMPAPGAIFDQSKYLMLYNDNKTVSSSAIAIEVHLTKK

SEQ ID NO: 29 - protein, 210 aa, Streptococcus pyrogenes
DENLKDLKRSLRFAYNITPCDYENVEIAFVTTNSIHINTKQKRSECILYVDSIVSLGITDQFIKGDKVDVFGLPYNFSPPYVDNIYGGIVKHSNQGNKSLQFVGILNQDGKETYLPSEVVRIKKKQFTLQEFDFKIRKFLMEKYNIYDSESRYTSGSLFLATKDSKHYEVDLFNKDDKLLSRDSFFKRYKDNKIFNSEEISHFDIYLKTY

SEQ ID NO: 30 - protein, 236 aa, Streptococcus pyrogenes
MRYNCRYSHIDKKIYSMIICLSFLLYSNVVQANSYNTTNRHNLESLYKHDSNLIEADSIKNSPDIVTSHMLKYSVKDKNLSVFFEKDWISQEFKDKEVDIYALSAQEVCECPGKRYEAFGGITLTNSEKKEIKVPVNVWDKSKQQPPMFITVNKPKVTAQEVDIKVRKLLIKKYDIYNNREQKYSKGTVTLDLNSGKDIVFDLYYFGNGDFNSMLKIYSNNERIDSTQFHVDVSIS

SEQ ID NO: 31 - protein, 209 aa, Streptococcus pyrogenes
LEVDNNSLLRNIYSTIVYEYSDTVIDFKTSHNLVTKKLDVRDARDFFINSEMDEYAANDFKAGDKIAVFSVPFDWNYLSKGKVTAYTYGGITPYQKTSIPKNIPVNLWINRKQIPVPYNQISTNKTTVTAQEIDLKVRKFLIAQHQLYSSGSSYKSGKLVFHTNDNSDKYSLDLFYTGYRDKESIFKVYKDNKSFNIDKIGHLDIEIDS

SEQ ID NO: 32 - protein, 209 aa, Streptococcus pyrogenes
GLEVDNNSLLRNIYSTIVYEYSDIVIDFKTSHNLVTKKLDVRDARDFFINSEMDEYAANDFKTGDKIAVFSVPFDWNYLSKGKVTAYTYGGITPYQKTSIPKNIPVNLWINGKQISVPYNEISTNKTTVTAQEIDLKVRKFLIAQHQLSSGSSYKSGRLVFHTNDNSDKYSFDLFYVGYRDKESIFKNYKDNKSFNIDKIGHLDIEIDS

SEQ ID NO: 33 - protein, 150 aa, Yersinia sp.
MKKKFLSLLTLTFFSGLALAADYDNTLNSIPSLRIPNIETYTGTIQGKGEVCIRGNKEGKSRGGELYAVLRSTNANADMTLILLCSIRDGWKEVKRSDIDRPLRYEDYYTPGALSWIWEIKNNSSEASDYSLSATVHDDKEDSDVLMKCP

SEQ ID NO: 34 - protein, 231 aa, Staphylococcus sp.
MKLKTLAKATLALSLLTTGVITLESQAVKAAEKQERVQHLYDIKDLYRYYSAPSFEYSNISGKVENYNGSNVVRFNQKDQNHQLFLLGKDKEQYKEGLQGKDVFVVQELIDPNGRLSTVGGVTKKNNKTSETKTHLLVNKVDGGNLDASIDSFLIQKEEISLKELDFKIRQQLVEKYGLYQGTSKYGKITINLKDEKREVIDLSDKLEFERMGDVLNSKDIKGISVTINQI

SEQ ID NO: 35 - protein, 231 aa, Staphylococcus sp.
MKLKTLAKATLALGLLTTGVITSEGQAVQAAEKQERVQHLHDIRDLHRYYSSESFEYSNVSGKVENYNGSNVVRFNPKDQNHQLFLLGKDKEQYKEGLQGQNVFVVQELIDPNGRLSTVGGVTKKNNKTSETNTPLFVNKVNGEDLDASIDSFLIQKEEISLKELDFKIRQQLVNNYGLYKGTSKYGKIIINLKDENKVEIDLGDKLQFERMGDVLNSKDIRGISVTINQI

SEQ ID NO: 36 - protein, 233 aa, Staphylococcus sp.
MKMTAIAKASLALSILATGVITSTAQTVNASEHESKYENVKDIFDKRDTYSRASKELKNVTGYRSKGGKKHYLIFDKNRKFTRIQIFGKDIERIKKRKNPGLDIFVVKEAENRNGTVYSYGGVTLLMQGAYYDYLSAPRFVIKKEVGAGVSVHVKRYYIYKEEISLKELDFKLRQYLIQDFDLYKKFPKASKIKVTMKDGGYYTFELNKKLQTNRMSDVIDGRNIEKIEANIR

SEQ ID NO: 37 - protein, 227 aa, Staphylococcus sp.
MKLTALAKVTLALGILTTGTLTTEAHSGHAKQNQKSVNKHDKEALHRYYTGNFKEMKNINALRHGKNNLRFKYRGMKTQVLLPBDEYRKYQQRRHTGLDVFFNQERRDKHDISYTVGGVTKTNKTSGFVSTPRLNVTKEKGEDAFVKGYPYDIKKEEISLKELDFKLRKHLIEKYGLYKTLSKDGRIKISLKDGSFYNLDLRTKLKFKHMGEVIDSKQIKDIEVNLK

SEQ ID NO: 38 - protein, 232 aa, Staphylococcus sp.
MKLTAIAKATLALGILTTGVMTAESQTVNAKVKLDETQRKYYINMLKDYYSQESYESTNISVKSEDYYGSNVLNFNQRNKNFKVFLIGDDRNKYKELTHGRDVFAVPELIDTKGGIYSVGGITKKNVRSVFGYVSHPGLQVKKVDPKDGFSIKELFFIQKEEVSLKELDFKIRKMLVEKYRLYKGASDKGRIVINMKDEKKHEIDLSEKLSFDRMFDVLDSKQIKNIEVNLN

SEQ ID NO: 39 - DNA, 26 bp, Artificial Sequence
  EPO HRE consensus sequence
ccgggtagctggcgtacgtgctgcag

SEQ ID NO: 40 - DNA, 1662 bp, Morbillivirus measles virus
gtgtccatcatggatctcaaggtgaacgtctctgccatattcatggcagtactgttaactctccaaacacccaccggtcaaatccattggggcaatctctctaagataggggtagtagggataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcctcaagtaggagacacaagagatttgcgggagttgtcctggcaggtgcggccctaggcgttgccacagctgctcagataacagccggcattgcacttcaccagtccatgctgaactctcaagccatcgacaatctgagagcgagcctggaaactaccaatcaggcaattgaggcaatcagacaagcagggcaggagatgatattggctgttcagggtgtccaagactacatcaataatgagctgataccgtctatgaaccaactatcttgtgatttaatcggccagaagctagggctcaaattgctcagatactatacagaaatcctgtcactatttggccccagcttacgggaccccatatctgcggagatatctatccaggctttgagctatgcgcttggaggagatatcaacaaggtgttagaaaagctcggatacagtggaggtgacttactgggcatcttagagagcaggggaataaaagcccggataactcacgtcgacacagagtcctacttcattgtactcagtatagcctatccgacgctgtccgagattaagggggtgattgtccaccggctagagggggtctcgtacaacataggctcccaagagtggtataccactgtgcccaagtatgttgcaacccaagggtacctcatctcgaattttgatgaatcatcgtgtactttcatgccagagggaactgtgtgcagccaaaatgccttgtacccaatgagtcctctgctccaagaatgcctccgggggtccaccaagtcctgtgctcgtacactcgtatccgggtcttttgggaaccggttcattttatcacaagggaatctaatagccaattgtgcatcaatcctttgcaagtgttacacaacaggaacgatcattaatcaagaccctgacaagatcctaacatacattgctgccgatcactgcccggtggtcgaggtgaacggcgtgaccatccaagtcgggagcaggagatatccggacgctgtgtacttgcacagaattgacctcggtcctcccatatcattggagaggttggacgtagggacaaatctggggaatgcaattgccaagttggaggacgccaaggaattgttggagtcatcggaccagatattgaggagtatgaaaggcttatcgagcactagcatagtttacatcctgattgcagtgtgtcttggagggctgatagggatccccgctttaatatgttgctgcagggggcgttgtaacaaaaagggagaacaagttggtatgtcaagaccaggcctaaagcctgatcttacaggaacatcaaaatcctatgtaaggtcgctctga

SEQ ID NO: 41 - protein, 550 aa, Morbillivirus measles virus
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGRCNKKGEQVGMSRPGLKPDLTGTSKSYVRSL

SEQ ID NO: 42 - DNA, 21 bp, Unknown
  primer
tcgtcttcaagaattctcatg

SEQ ID NO: 43 - DNA, 20 bp, Unknown
  primer
tttcagtcaccggtgtcgga

SEQ ID NO: 44 - DNA, 20 bp, Unknown
  primer
tccgacaccggtgactgaaa

SEQ ID NO: 45 - DNA, 20 bp, Unknown
  primer
gcattctctagacacaggtg

SEQ ID NO: 46 - DNA, 35 bp, Unknown
  primer
atttagcggccgcagttcacaaccgaaatgtcttc

SEQ ID NO: 47 - DNA, 29 bp, Unknown
  primer
agtttaccggtatcctgcacctcgcgctg

SEQ ID NO: 48 - DNA, 30 bp, Unknown
  primer
tccccgcggtaaatgtgctgtctttagaag

SEQ ID NO: 49 - DNA, 32 bp, Unknown
  primer
aatatgcggccgctccaataggaaagcccttc

SEQ ID NO: 50 - DNA, 21 bp, Unknown
  primer
tcacctgtgtctagagaatgc

SEQ ID NO: 51 - DNA, 20 bp, Unknown
  primer
gtaaccaagcttagcccacg

SEQ ID NO: 52 - DNA, 20 bp, Unknown
  primer
cgtgggctaagcttggttac

SEQ ID NO: 53 - DNA, 21 bp, Unknown
  primer
ccagaaaatccagcaggtacc

SEQ ID NO: 54 - DNA, 34 bp, Unknown
  primer
gatcatgctaagcgatcccagcgctaccatcttc

SEQ ID NO: 55 - DNA, 32 bp, Unknown
  primer
tataatgcttagcgtgggggcctgtgcgctgg

SEQ ID NO: 56 - protein, 5 aa, Pseudomonas sp.
REDLK

SEQ ID NO: 57 - protein, 613 aa, Pseudomonas sp.
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 58 - protein, 4 aa, Pseudomonas sp.
REDL

SEQ ID NO: 59 - protein, 4 aa, Pseudomonas sp.
KDEL

SEQ ID NO: 60 - protein, 5 aa, Corynebacterium diphtheriae
GADDV

SEQ ID NO: 61 - DNA, 31 bp, Unknown
  primer
ctagtctagaatggtattgctgcctgggtcc

SEQ ID NO: 62 - DNA, 34 bp, Unknown
  primer
atatcggcatgcacatgcacttatcctatcattg

SEQ ID NO: 63 - protein, 319 aa, Staphylococcus sp.
MKTRIVSSVTTTLLLGSILMNPVAGAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 64 - protein, 272 aa, Listeria sp.
MTIKKERADILLVEQGLFETREKAKRAIMAGIVYRKEERVDKPGEKIPIDELQVKGKQMPYVSRGGLKLEKALQVFDFEVKDKLMLDIGASTGGFTDCALQNGARHSYALDVGYNQLAWKLRNDDRVTVMERTNFRHVTPADFSEGLADFATIDVSFISLKLILPVLRTVLVTGGDVMTLIKPQFEAGREQVGKKGIIRDPAVHESVVEHIVQFALDNGYDLMGLDFSPITGGEGNIEFAHLKWTGGETGTNHLEPNAIAKLITKAHTKLDK

SEQ ID NO: 65 - protein, 312 aa, Staphylococcus aureus
MVKKRLLAATLSLGIITPIATSFHESKADNNIENIGDGAEVVKRTEDTSSDKWGVTQNIQFDFVKDKKYNKDALILKMQGFINSKTTYYNYKNTDHIKAMRWPFQYNIGLKTNDPNVDLINYLPKNKIDSVNVSQTLGYNIGGNFNSGPSTGGNGSFNYSKTISYNQQNYISEVEHQNSKSVQWGIKANSFITSLGKMSGHDPNLFVGYKPYSQNPRDYFVPDNELPPLVHSGFNPSFIATVSHEKGSGDTSEFEITYGRNMDVTHATRRTTHYGNSYLEGSRIHNAFVNRNYTVKYEVNWKTHEIKVKGHN

SEQ ID NO: 66 - protein, 628 aa, Homo sapiens
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 67 - protein, 271 aa, Homo sapiens
MGSLFPLSLLFFLAAAYPGVGSALGRRTKRAQSPKGSPLAPSGTSVPFWVRMSPEFVAVQPGKSVQLNCSNSCPQPQNSSLRTPLRQGKTLRGPGWVSYQLLDVRAWSSLAHCLVTCAGKTRWATSRITAYKPPHSVILEPPVLKGRKYTLRCHVTQVFPVGYLVVTLRHGSRVIYSESLERFTGLDLANVTLTYEFAAGPRDFWQPVICHARLNLDGLVVRNSSAPITLMLAWSPAPTALASGSIAALVGILLTVGAAYLCKCLAMKSQA

SEQ ID NO: 68 - protein, 1032 aa, Homo sapiens [sequence omitted]

SEQ ID NO: 69 - DNA, 25 bp, Unknown
  primer
tttagaagatgaaccgtaagcctag

SEQ ID NO: 70 - DNA, 21 bp, Unknown
  primer
gatacctctttcgtctgctga

SEQ ID NO: 71 - DNA, 40 bp, Unknown
  primer
tctagacgcgtagatctcacgtgagcatgcaggccttggg

SEQ ID NO: 72 - DNA, 38 bp, Unknown
  primer
cgcgtaaagcatctctacggtggtcctaatagtgcatg

SEQ ID NO: 73 - DNA, 30 bp, Unknown
  primer
cactattaggaccaccgtcgagatgcttta

SEQ ID NO: 74 - DNA, 36 bp, Unknown
  primer
acatccatccactagtgcccccgttgacattgatta

SEQ ID NO: 75 - DNA, 36 bp, Unknown
  primer
acatgcatgcatgtgagagctctgcttatatagacc